(19) United States
(12) Patent Application Publication
(10) Pub. No.: US 2010/0068181 A1
(43) Pub. Date: Mar. 18, 2010

(54) PYRROLO [3, 2-A] PYRIDINE DERIVATIVES FOR INHIBITING KSP KINESIN ACTIVITY

(75) Inventors: Sunil Paliwal, Monroe Township, NJ (US); Hon-Chung Tsui, East Brunswick, NJ (US); Jose S. Duca, Cranford, NJ (US); Charles A. Lesburg, Short Hills, NJ (US); Ronald J. Doll, Convent Station, NJ (US); Neng-Yang Shih, Lexington, MA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/519,740

(22) PCT Filed: Dec. 19, 2007
(86) PCT No.: PCT/US2007/026065

§ 371 Date: Nov. 25, 2009

Related U.S. Application Data
(60) Provisional application No. 60/876,501, filed on Dec. 21, 2006.

(51) Int. Cl. A61K 38/21; C07D 471/02; A61K 31/437; C07D 401/14; A61K 31/496; A61K 31/506; A61K 31/66; A61K 31/70; A61K 38/14; A61K 31/704; A61K 38/46; A61K 31/56; A61K 33/24; A61K 39/395; A61P 35/00

(52) U.S. Cl. 424/85.4; 546/84; 514/292; 544/361; 514/253.03; 544/331; 514/275; 514/110; 514/25; 514/8; 514/34; 424/94.6; 514/178; 424/649; 424/143.1

(57) ABSTRACT

The present invention provides compounds of Formula I (wherein R, R¹, R³, R⁴, X, and ring Y are as defined herein). The present invention also provides compositions comprising these compounds that are useful for treating cellular proliferative diseases or disorders associated with KSP kinesin activity and for inhibiting KSP kinesin activity.

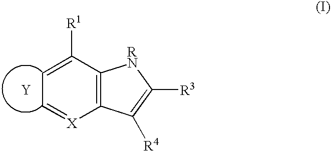

### FIELD OF THE INVENTION

The present invention relates to compounds and compositions that are useful for treating cellular proliferative diseases or disorders associated with Kinesin Spindle Protein (“KSP”) kinesin activity and for inhibiting KSP kinesin activity.

### BACKGROUND OF THE INVENTION

Cancer is a leading cause of death in the United States and throughout the world. Cancer cells are often characterized by constitutive proliferative signals, defects in cell cycle checkpoints, as well as defects in apoptotic pathways. There is a great need for the development of new chemotherapeutic drugs that can block cell proliferation and enhance apoptosis of tumor cells.

Conventional therapeutic agents used to treat cancer include taxanes and vinca alkaloids, which target microtubules. Microtubules are an integral structural element of the mitotic spindle, which is responsible for the distribution of the duplicated sister chromatids to each of the daughter cells that result from cell division. Disruption of microtubules or interference with microtubule dynamics can inhibit cell division and induce apoptosis.

However, microtubules are also important structural elements in non-proliferative cells. For example, they are required for organelle and vesicle transport within the cell or along axons. Since microtubule-targeted drugs do not discriminate between these different structures, they can have undesirable side effects that limit usefulness and dosage. There is a need for chemotherapeutic agents with improved specificity to avoid side effects and improve efficacy.

Microtubules rely on two classes of motor proteins, the kinesins and dyneins, for their function. Kinesins are motor proteins that generate motion along microtubules. They are characterized by a conserved motor domain, which is approximately 320 amino acids in length. The motor domain binds and hydrolyses ATP as an energy source to drive directional movement of cellular cargo along microtubules and also contains the microtubule binding interface (Mandelkow and Mandelkow, Trends Cell Biol. 2002, 12:585-591).

Kinesins exhibit a high degree of functional diversity, and several kinesins are specifically required during mitosis and cell division. Different mitotic kinesins are involved in all aspects of mitosis, including the formation of a bipolar spindle, spindle dynamics, and chromosome movement. Thus, interference with the function of mitotic kinesins can disrupt normal mitosis and block cell division. Specifically, the mitotic kinesin KSP (also termed EG5), which is required for centrosome separation, was shown to have an essential function during mitosis. Cells in which KSP function is inhibited arrest in mitosis with unseparated centrosomes (Blangy et al., Cell 1995, 83:1159-1169). This leads to the formation of a monoastral array of microtubules, at the end of which the duplicated chromatids are attached in a rosette-like configuration. Further, this mitotic arrest leads to growth inhibition of tumor cells (Kaiser et al., J. Biol. Chem. 1999, 274:18925-18931). Inhibitors of KSP would be desirable for the treatment of proliferative diseases, such as cancer.

Kinesin inhibitors are known, and several molecules have recently been described in the literature. For example, adociasulfate-2 inhibits the microtubule-stimulated ATPase activity of several kinesins, including CENP-E (Sakowicz et al., Science 1998, 280:292-295). Rose Bengal lactone, another non-selective inhibitor, interferes with kinesin function by blocking the microtubule binding site (Hopkins et al., Biochemistry 2000, 39:2805-2814). Monastrol, a compound that has been isolated using a phenotypic screen, is a selective inhibitor of the KSP motor domain (Mayer et al., Science 1999, 286:971-974). Treatment of cells with monastrol arrests cells in mitosis with monopolar spindles.

WO2006/098961 and WO2006/098962 disclose compounds that are useful for treating cellular proliferative diseases or disorders associated with KSP kinesin activity and for inhibiting KSP kinesin activity.

KSP, as well as other mitotic kinesins, are attractive targets for the discovery of novel chemotherapeutics with anti-proliferative activity. There is a need for compounds useful in the inhibition of KSP, and in the treatment of proliferative diseases, such as cancer.

### SUMMARY OF THE INVENTION

In one embodiment, the present invention provides a compound represented by the structural Formula (I):

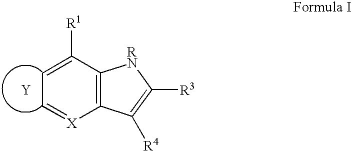

or a pharmaceutically acceptable salt, solvate or ester thereof, wherein:

ring Y is a 3- to 7-membered cycloalkyl or cycloalkenyl fused as shown in Formula I, wherein each of said 3- to 7-membered cycloalkyl or cycloalkenyl, is optionally substituted with 1-2 R² moieties;

X is N or N-oxide;

R and R¹ are each independently selected from the group consisting of selected from the group consisting of H, halo, alkyl, cycloalkyl, cycloalkylalkyl, heterocyclyl, heterocyclylalkyl, aryl, aralkyl, heteroaryl, heteroaralkyl, —(CR¹¹R¹²)₀₋₆—OR⁸, —C(O)R⁵, —C(S)R⁵, —C(O)OR⁸, —C(S)OR⁸, —OC(O)R⁸, —OC(S)R⁸, —C(O)NR⁵R⁶, —C(S)NR⁵R⁶, —C(O)NR⁵OR⁸, —C(S)NR⁵OR⁸, —C(O)NR⁸NR⁵R⁶, —C(S)NR⁸NR⁵R⁸, —C(S)NR⁵OR⁸, —C(O)SR⁸, —NR⁵R⁶, —NR⁵C(O)R⁶, —NR⁵C(S)R⁶, —NR⁵C(O)OR⁸, —NR⁵C(S)OR⁸, —OC(O)NR⁵R⁶, —OC(S)NR⁵R⁶, —NR⁵C(O)NR⁵R⁶, —NR⁵C(S)NR⁵R⁶, —NR⁵C(O)NR⁵OR⁸, —NR⁵C(S)NR⁵OR⁸, —(CR¹¹R¹²)₀₋₆SR⁸, SO₂R⁸, —S(O)₁₋₂NR⁵R⁶, —N(R⁸)SO₂R⁸, —S(O)₁₋₂NR⁶OR⁸, —CN, —OCF₃, —SCF₃, —C(═NR⁸)NR⁵, —C(O)NR⁸(CH₂)₁₋₁₀NR⁵R⁶, —C(O)NR⁸(CH₂)₁₋₁₀OR⁸, —C(S)NR⁸(CH₂)₁₋₁₀NR⁵R⁶, —C(S)NR⁸(CH₂)₁₋₁₀OR⁸, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R¹⁰ moieties;

each R² is independently selected from the group consisting of H, halo, alkyl, cycloalkyl, alkylsilyl, cycloalkenyl, heterocyclyl, heterocyclenyl, aryl, heteroaryl, —(CR¹¹R¹²)₀₋₆—OR⁸, —C(O)R⁵, —C(S)R⁵, —C(O)OR⁸, —C(S)OR⁸, —OC(O)R⁸, —OC(S)R⁸, —C(O)NR⁵R⁶, —C(S)NR⁵R⁶, —C(O)NR⁵OR⁸, —C(S)NR⁵OR⁸, —C(O)NR⁸NR⁵R⁶, —C(S)NR⁸NR⁵R⁶, —C(S)NR⁵OR⁸, —C(O)SR⁸, —NR⁵R⁶, —NR⁵C(O)R⁶, —NR⁵C(S)R⁶, —NR⁵C(O)OR⁸, —NR⁵C(S)OR⁸, —OC(O)NR⁵R⁶, —OC(S)NR⁵R⁶, —NR⁵C(O)NR⁵R⁶, —NR⁵C(S)NR⁵R⁶, —NR⁵C(O)NR⁵OR⁸, —NR⁵C(S)NR⁵OR⁸, —(CR¹¹R¹²)₀₋₆SR⁸, SO₂R⁸, —S(O)₁₋₂NR⁵R⁶, —N(R⁸)SO₂R⁸, —S(O)₁₋₂NR⁶OR⁸, —CN, —OCF₃, —SCF₃, —C(═NR⁸)NR⁵, —C(O)NR⁸(CH₂)₁₋₁₀NR⁵R⁶, —C(O)NR⁸(CH₂)₁₋₁₀OR⁸, —C(S)NR⁸(CH₂)₁₋₁₀NR⁵R⁶, and —C(S)NR⁸(CH₂)₁₋₁₀OR⁸, wherein each of said alkyl, cycloalkyl, cycloalkenyl, heterocyclyl, heterocyclenyl, aryl, and heteroaryl is independently optionally substituted with 1-5 R¹⁰ moieties;

or two R²s on the same carbon atom are optionally taken together with the carbon atom to which they are attached to form a C═O, a C═S or an ethylenedioxy group;

R³ and R⁴ are each independently selected from the group consisting of H, halo, hydroxy, nitro, alkyl, alkenyl, alkynyl, alkoxy, cycloalkyl, cycloalkenyl, heterocyclyl, heterocyclenyl, aryl, heteroaryl, —C(O)R⁵, —C(S)R⁵, —C(O)OR⁸, —C(S)OR⁸, —OC(O)R⁸, —OC(S)R⁸, —C(O)NR⁵R⁶, —C(S)NR⁵R⁶, —C(O)NR⁵OR⁸, —C(S)NR⁵OR⁸, —C(O)NR⁸NR⁵R⁶, —C(S)NR⁸NR⁵R⁶, —C(S)NR⁵OR⁸, —C(O)SR⁸, —NR⁵R⁶, —NR⁵C(O)R⁶, —NR⁵C(S)R⁶, —NR⁵C(O)OR⁸, —NR⁵C(S)OR⁸, —OC(O)NR⁵R⁶, —OC(S)NR⁵R⁶, —NR⁵C(O)NR⁵R⁶, —NR⁵C(S)NR⁵R⁶, —NR⁵C(O)NR⁵OR⁸, —NR⁵C(S)NR⁵OR⁸, —(CR¹¹R¹²)₀₋₆SR⁸, SO₂R⁸, —S(O)₁₋₂NR⁵R⁶, —N(R⁸)SO₂R⁸, —S(O)₁₋₂NR⁶OR⁸, —CN, —C(═NR⁸)NR⁵R⁶, —C(═NOR⁸)R⁵, —C═N—N(R⁸)—C(═S)NR⁵R⁶, —C(O)N(R⁸)—(CR⁴⁰R⁴¹)₁₋₅—C(═NR⁸)NR⁵R⁶, —C(O)N(R⁸)(CR⁴⁰R⁴¹)₁₋₅—NR⁵R⁶, —C(O)N(R⁸)(CR⁴⁰R⁴¹)₁₋₅—C(O)—NR⁵R⁶, —C(O)N(R⁸)(CR⁴⁰R⁴¹)₁₋₅—OR⁸, —C(S)NR⁸(CH₂)₁₋₅NR⁵R⁶, and —C(S)NR⁸(CH₂)₁₋₅OR⁸, wherein each of said alkyl, alkenyl, alkynyl, cycloalkyl, cycloalkenyl, heterocyclyl, heterocyclenyl, aryl, and heteroaryl is independently optionally substituted with 1-5 R¹⁰ moieties;

each of R⁵ and R⁶ is independently selected from the group consisting of H, alkyl, alkenyl, alkynyl, cycloalkyl, cycloalkenyl, heterocyclyl, heterocyclenyl, aryl, heteroaryl, —OR⁸, —C(O)R⁸, and —C(O)OR⁸, with the proviso that R⁵ and R⁶ are not simultaneously —OR⁸; wherein each of said alkyl, alkenyl, alkynyl, cycloalkyl, cycloalkenyl, heterocyclyl, heterocyclenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl;

each R⁸ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁸ except H is optionally substituted with 1-4 R⁹ moieties;

each R⁹ is independently selected from the group consisting of halo, alkyl, cycloalkyl, cycloalkenyl, heterocyclyl, heterocyclenyl, aryl, heteroaryl, —NO₂, —OR¹¹, —OC(═O)R¹¹, —(C₁-C₆ alkyl)-OR¹¹, —CN, —NR¹¹R¹², —C(O)R¹¹, —C(O)OR¹¹, —C(O)NR¹¹R¹², —CF₃, —OCF₃, —CF₂CF₃, —C(═NOH)R¹¹, —NR¹¹C(═O)R¹², —C(═NR¹¹)NR¹¹R¹², and —NR¹¹C(═O)OR¹²; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl; or two R⁹ groups, when attached to the same carbon, are optionally taken together with the carbon atom to which they are attached to form a C═O or a C═S group;

each R¹⁰ is independently selected from the group consisting of H, alkyl, heterocyclyl, aryl, alkoxy, OH, CN, halo, —(CR¹¹R¹²)₀₋₄NR⁵R⁶, haloalkyl, haloalkoxy, hydroxyalkyl, alkoxyalkyl, —O-alkyl-O-alkyl, —C(O)NR⁵R⁶, —C(O)OR⁸, —OC(O)R⁵, —OC(O)NR⁵R⁶, —NR⁵C(O)R⁶, —NR⁵C(O)OR⁶, —NR⁵C(O)NR⁵R⁶, —SR⁸, —S(O)R⁸, and —S(O)₂R⁸, wherein each of said alky, heterocyclyl and aryl is optionally independently substituted with 1-4 R¹³ moieties;

each R¹¹ is independently H or alkyl;

each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹³ is independently selected from the group consisting of H, halo, alkyl, alkylsilyl, alkoxy, haloalkyl, cyano, and hydroxy;

each R⁴² is independently selected from the group consisting of halo, alkyl, cycloalkyl, heterocyclyl, aryl, heteroaryl, —NO₂, —OR¹¹, —(C₁-C₆ alkyl)-OR¹¹,

—CN, —NR¹¹R¹², —C(O)R¹¹, —C(O)OR¹¹, —C(O)NR¹¹R¹², —CF₃, —OCF₃, —N(R¹¹)C(O)R¹², and —NR¹¹C(O)OR¹², wherein each of said aryl, heterocyclyl and heteroaryl is optionally substituted with 1-4 R⁴³ moieties; and

each R⁴³ is independently selected from the group consisting of halo, alkyl, alkoxy, haloalkyl, cyano, and hydroxyl;

with the proviso that R and R³ are optionally taken together, with the ring nitrogen and carbon atom to which they are respectively shown attached, to form a heteroaryl, heterocyclyl or heterocyclenyl ring that is optionally substituted with 1-3 moieties independently selected from the group consisting of oxo, thioxo, —OR¹², —NR¹¹R¹², —C(═O)R¹², —C(═O)OR¹², —C(═O)NR¹¹R¹², and —NR¹¹C(═O)R¹².

Pharmaceutical formulations or compositions for the treatment of cellular proliferative diseases, disorders associated with KSP kinesin activity and/or for inhibiting KSP kinesin activity in a subject comprising administering a therapeutically effective amount of at least one of the inventive compounds and a pharmaceutically acceptable carrier to the subject also are provided.

Methods of treating cellular proliferative diseases, disorders associated with KSP kinesin activity and/or for inhibiting KSP kinesin activity in a subject comprising administering to a subject in need of such treatment an effective amount of at least one of the inventive compounds also are provided.

Other than in the operating examples, or where otherwise indicated, all numbers expressing quantities of ingredients, reaction conditions, and so forth used in the specification and claims are to be understood as being modified in all instances by the term “about.”

### DETAILED DESCRIPTION

In one embodiment, the present invention discloses compounds represented by structural Formula I or a pharmaceutically acceptable salt or ester thereof, wherein the various moieties are as described above.

In another embodiment, in Formula (I), ring Y is a 3- to 7-membered cycloalkyl which is optionally substituted with 1-2 R² moieties.

In another embodiment, in Formula (I), ring Y is a 6-membered cycloalkyl, which is optionally substituted with 1-2 R² moieties.

In another embodiment, in Formula (I), ring Y is substituted with one R² moiety.

In another embodiment, in Formula (I), R² is alkyl.

In another embodiment, in Formula (I), R² is butyl.

In another embodiment, in Formula (I), R is selected from the group consisting of H and —C(O)R⁵.

In another embodiment, in Formula (I), R is selected from the group consisting of H and —C(O)R⁵, wherein R⁵ is alkyl.

In another embodiment, in Formula (I), R¹ is H.

In another embodiment, in Formula (I), R is H.

In another embodiment, in Formula (I):

R³ and R⁴ are each independently selected from the group consisting of H, halo, hydroxy, nitro, alkyl, alkenyl, alkynyl, alkoxy, heterocyclyl, aryl, heteroaryl, —C(O)R⁵, —C(O)OR⁸, —C(O)NR⁵R⁶, —C(O)NR⁸NR⁵R⁶, —NR⁵R⁶, —NR⁵C(O)R⁶, —N(R⁸)SO₂R⁸, —CN, —C(═NOR⁸)R⁵, and —C═N—N(R⁸)—C(═S)NR⁵R⁶, wherein each of said alkyl, alkenyl, alkynyl, heterocyclyl, and aryl is independently optionally substituted with 1-5 R¹⁰ moieties;

each of R⁵ and R⁶ is independently selected from the group consisting of H, alkyl, alkenyl, aryl, heterocyclyl, and heteroaryl wherein each of said alkyl, alkenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;

each R⁸ is independently alkyl, which is optionally substituted with 1-4 R⁹ moieties;

each R⁹ is independently selected from the group consisting of alkyl, heterocyclyl, aryl, heteroaryl, —OR¹¹, —OC(═O)R¹¹, —CN, —NR¹¹R¹², —NR¹¹C(═O)OR¹², —C(═O)NR¹¹R¹², —NR¹¹C(═O)R¹², and —C(O)OR¹¹; wherein each of said alkyl, heterocyclyl, aryl, and heteroaryl is independently optionally substituted with 1-4 R⁴² moieties; wherein when each of said heterocyclyl, aryl, and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;

each R¹⁰ is independently selected from the group consisting of H, alkyl, alkoxy, OH, CN, halo, heterocyclyl, aryl, heteroaryl, —O-alkyl-O-alkyl, —NR⁵R⁶, haloalkyl, haloalkoxy, hydroxyalkyl, alkoxyalkyl, —C(═O)NR⁵R⁶, —C(═O)OR⁸, —OC(═O)R⁵, —OC(═O)NR⁵R⁶, —NR⁵C(═O)R⁶, —NR⁵C(═O)OR⁶, —NR⁵C(═O)NR⁵R⁶, and —S(═O)₂R⁸, wherein each of said heterocyclyl, aryl, and heteroaryl moieties is optionally independently substituted with 1-4 R¹³ moieties;

each R¹¹ is independently H or alkyl; and

each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹³ is independently selected from the group consisting of halo, alkyl, alkoxy, haloalkyl, cyano, and hydroxy;

each R⁴² is independently selected from the group consisting of halo, alkyl, cycloalkyl, heterocyclyl, aryl, heteroaryl, —NO₂, —OR¹¹, —(C₁-C₆ alkyl)-OR¹¹,

—CN, —NR¹¹R¹², —C(═O)R¹¹, —C(═O)OR¹¹, —C(═O)NR¹¹R¹², —CF₃, —OCF₃, —NR¹¹C(═O)R¹², and —NR¹¹C(═O)OR¹², wherein each of said aryl, heterocyclyl and heteroaryl is optionally substituted with 1-4 R⁴³ moieties; and

each R⁴³ is independently selected from the group consisting of halo, alkyl, alkoxy, haloalkyl, cyano, and hydroxyl.

In another embodiment, in Formula (I):

wherein R³ is selected from the group consisting of H, halo, hydroxy, nitro, alkyl, alkenyl, alkoxy, —C(O)R⁵, —C(O)OR⁸, —C(O)NR⁵R⁶, —C(O)NR⁸NR⁵R⁶, —CN, —C(═NOR⁸)R⁵, and —C═N—N(R⁸)—C(═S)NR⁵R⁶, wherein each of said alkyl and alkenyl is independently optionally substituted with 1-5 R¹⁰ moieties;

each of R⁵ and R⁶ is independently selected from the group consisting of H, alkyl, alkenyl, aryl, heterocyclyl, and heteroaryl wherein each of said alkyl, alkenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;

each R⁸ is independently alkyl, which is optionally substituted with 1-4 R⁹ moieties;

each R⁹ is independently selected from the group consisting of alkyl, aryl, heteroaryl, —OR¹¹, —OC(═O)R¹¹, —CN, —NR¹¹R¹², and —C(O)OR¹¹; wherein said each of said alkyl, aryl, and heteroaryl is independently optionally substituted with 1-4 R⁴² moieties; wherein when each of said aryl and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;

each R¹⁰ is independently selected from the group consisting of alkoxy, OH, haloalkoxy, heterocyclyl, aryl, —NR⁵R⁶, —CN, —OC(═O)R⁵, and —O-alkyl-O-alkyl, wherein each of said heterocyclyl and aryl is optionally independently substituted with 1-4 R¹³ moieties;

each R¹¹ is independently H or alkyl; and

each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹³ is independently selected from the group consisting of halo, alkyl, alkoxy, haloalkyl, cyano, and hydroxyl;

each R⁴² is independently selected from the group consisting of halo, alkyl, cycloalkyl, heterocyclyl, aryl, heteroaryl, —NO₂, —OR¹¹, —(C₁-C₆ alkyl)-OR¹¹,

—CN, —NR¹¹R¹², —C(═O)R¹¹, —C(═O)OR¹¹, —C(═O)NR¹¹R¹², —CF₃, —OCF₃, —NR¹¹C(═O)R¹², and —NR¹¹C(═O)OR¹², wherein each of said aryl, heterocyclyl and heteroaryl is optionally substituted with 1-4 R⁴³ moieties;
each R⁴³ is independently selected from the group consisting of halo, alkyl, alkoxy, haloalkyl, cyano, and hydroxyl.

In another embodiment, in Formula (I), R³ is selected from the group consisting of H, alkyl, alkenyl, halo, hydroxyl, cyano, H₂NNH—C(═O)—, alkyl-NH—NH—(C═O)—, heteroaryl-NH—NH—C(═O)—, aryl-alkyl-, alkoxy, NH₂-alkyl-, NC-alkyl-, aryl-C(═O)—O-alkyl-, alkyl-O—C(═O)—, H₂N—C(═O)—, aryl-NH—NH—C(═O)—, aryl-NH—C(═O)—, heteroaryl-NH—C(═O)—, alkyl-C(═O)—, alkyl-NH—C(═O)—, aryl-alkyl-NH—C(═O)—, HO-alkyl-aryl-NH—C(═O)—, heteroaryl-alkyl-NH—C(═O)—, heterocyclyl-alkyl-NH—C(═O)—, H₂N-alkyl-NH—C(═O)—, HO-alkyl-NH—C(═O)—, alkyl-O-alkyl-, NC-alkyl-NH—NH—C(═O)—, alkyl-O-alkyl-O-alkyl-, H₂N—C(═S)—NH—N═CH—, alkyl-C(═NOH)—, and heterocyclyl-C(═O)—; wherein each of said alkyl, alkenyl, and the “alkyl” part of aryl-alkyl- and aryl-alkyl-NH—C(═O)— is optionally substituted with 1-2 moieties selected from the group consisting of hydroxy and NH₂; wherein the “aryl” part of each of said aryl-alkyl-, aryl-NH—C(═O)—, and aryl-alkyl-NH—C(═O)— is optionally substituted with 1-2 moieties selected from the group consisting of halo, alkoxy, hydroxyl, NH₂, and heteroaryl-C(═O)—NH—; and wherein when the “aryl” part of any of said R³ groups contains two adjacent moieties, such moieties have optionally be taken together with the carbon atoms to which they are attached to a form a five to six membered heterocyclyl or heteroaryl.

In another embodiment, in Formula (I):

R⁴ is selected from the group consisting of H, halo, nitro, alkyl, alkenyl, alkynyl, heterocyclyl, aryl, —C(═O)R⁵, —C(═O)OR⁸, —C(═O)NR⁵R⁶, —C(═O)NR⁸NR⁵R⁶, —NR⁵R⁶, —NR⁵C(═O)R⁶, —NR⁸SO₂R⁸, wherein each of said alkyl, alkenyl, alkynyl, heterocyclyl, and aryl is independently optionally substituted with 1-5 R¹⁰ moieties;

each of R⁵ and R⁶ is independently selected from the group consisting of H, alkyl, alkenyl, and heteroaryl wherein each of said alkyl, alkenyl, and heteroaryl is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;

each R⁸ is independently alkyl, which is optionally substituted with 1-4 R⁹ moieties;

each R⁹ is independently selected from the group consisting of alkyl, heterocyclyl, aryl, heteroaryl, —OC(═O)R¹¹, —CN, —NR¹¹R¹², —NR¹¹C(═O)OR¹², —C(═O)NR¹¹R¹², —NC(═O)R¹², and —C(═O)OR¹¹; wherein said each of said alkyl, heterocyclyl, and heteroaryl is independently optionally substituted with 1-4 R⁴² moieties; wherein when each of said heterocyclyl and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;

each R¹⁰ is independently selected from the group consisting of H, alkyl, alkoxy, OH, CN, —O-alkyl-O-alkyl, —NR⁵R⁶, haloalkoxy, —C(═O)NR⁵R⁶, —NR⁵C(═O)R⁶, —NR⁵C(═O)OR⁶, and —S(═O)₂R⁸;

each R⁴² is independently selected from the group consisting of halo, alkyl, cycloalkyl, heterocyclyl, aryl, heteroaryl, —NO₂, —OR¹¹, —(C₁-C₆ alkyl)-OR¹¹,

—CN, —NR¹¹R¹², —C(═O)R¹¹, —C(═O)OR¹¹, —C(═O)NR¹¹R¹², —CF₃, —OCF₃, —N(R¹¹)C(═O)R¹², and —NR¹¹C(═O)OR¹², wherein each of said aryl, heterocyclyl and heteroaryl is optionally substituted with 1-4 R⁴³ moieties;

each R¹¹ is independently H or alkyl; and

each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl; and

each R⁴³ is independently selected from the group consisting of halo, alkyl, alkoxy, haloalkyl, cyano, and hydroxyl.

In another embodiment, in Formula (I), R⁴ is selected from the group consisting of H, halo, nitro, H₂N—, alkyl, HO-alkyl-, (HO)₂alkyl-, alkyl-C(═O)-alkyl-C(═O)—NH—, alkenyl-C(═O)-alkyl-C(═O)—NH—, H₂N—C(═O)-alkyl-whose “alkyl” is optionally substituted with an alkyl-C(═O)—NH—, NC-alkyl-, H₂N-alkyl-, alkyl-O—C(═O)—NH—, HO—C(═O)—NH—, alkyl-C(═O)—O-alkyl-C(═O)—NH—, alkyl-O—C(═O)-alkenyl-, heteroaryl-C(═O)—NH—, heterocyclyl, HO-alkynyl-, alkyl-O-alkyl-NH—, HO-alkyl-NH—, alkyl-S(═O)₂NH—, alkyl-O—C(═O)—, HO-alkyl-NH—C(═O)—, (HO)₂alkyl-NH—C(═O)—, H₂N-alkyl-NH—C(═O)—, heterocyclyl-alkyl-NH—C(═O)—, heteroaryl-alkyl-NH—C(═O)—, alkenyl-NH—C(═O)—, H₂N—NH—C(═O)—, H₂N—C(═O)—, alkyl-C(═O)—NH—, heteroaryl-C(═O)—, heteroaryl-NH—C(═O)—, and aryl that is optionally substituted with 1-2 moieties selected from the group consisting of hydroxy, alkoxy, haloalkoxy, cyano, H₂N—, and alkyl-S(═O)—.

In another embodiment, in Formula (I):

X is N;

ring Y is a 6-membered cycloalkyl which is substituted with an alkyl;

R is selected from the group consisting of H and —C(O)R⁵;

R¹ is H;

R³ is selected from the group consisting of H, halo, hydroxy, nitro, alkyl, alkenyl, alkoxy, —C(O)R⁵, —C(O)OR⁸, —C(O)NR⁵R⁶, —C(O)NR⁸NR⁵R⁶, —CN, —C(═NOR⁸)R⁵, and —C═N—N(R⁸)—C(═S)NR⁵R⁶, wherein each of said alkyl and alkenyl is independently optionally substituted with 1-5 R¹⁰ moieties; and

R⁴ is selected from the group consisting of H, halo, nitro, alkyl, alkenyl, alkynyl, heterocyclyl, aryl, —C(═O)R⁵, —C(═O)OR⁸, —C(═O)NR⁵R⁶, —C(═O)NR⁸NR⁵R⁶, —NR⁵R⁶, —NR⁵C(═O)R⁶, —NR⁸SO₂R⁸, wherein each of said alkyl, alkenyl, alkynyl, heterocyclyl, and aryl is independently optionally substituted with 1-5 R¹⁰ moieties;

each of R⁵ and R⁶ is independently selected from the group consisting of H, alkyl, alkenyl, aryl, heterocyclyl, and heteroaryl wherein each of said alkyl, alkenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;

each R⁸ is independently alkyl, which is optionally substituted with 1-4 R⁹ moieties;

each R⁹ is independently selected from the group consisting of alkyl, heterocyclyl, aryl, heteroaryl, —OR¹¹, —OC(═O)R¹¹, —CN, —NR¹¹R¹², —NR¹¹C(═O)OR¹², —C(═O)NR¹¹R₁₂, —NR¹¹C(═O)R¹², and —C(O)OR¹¹; wherein each of said alkyl, heterocyclyl, aryl, and heteroaryl is independently optionally substituted with 1-4 R⁴² moieties; wherein when each of said heterocyclyl, aryl, and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;

each R¹⁰ is independently selected from the group consisting of H, alkyl, alkoxy, OH, CN, halo, heterocyclyl, aryl, heteroaryl, —O-alkyl-O-alkyl, —NR⁵R⁶, haloalkyl, haloalkoxy, hydroxyalkyl, alkoxyalkyl, —C(═O)NR⁵R⁶, —C(═O)OR⁸, —OC(═O)R⁵, —OC(═O)NR⁵R⁶, —NR⁵C(═O)R⁶, —NR⁵C(═O)OR⁶, —NR⁵C(═O)NR⁵R⁶, and —S(═O)₂R⁸, wherein each of said heterocyclyl, aryl, and heteroaryl moieties is optionally independently substituted with 1-4 R¹³ moieties;

each R¹¹ is independently H or alkyl; and

each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;

each R¹³ is independently selected from the group consisting of halo, alkyl, alkoxy, haloalkyl, cyano, and hydroxy;

each R⁴² is independently selected from the group consisting of halo, alkyl, cycloalkyl, heterocyclyl, aryl, heteroaryl, —NO₂, —OR¹¹, —(C₁-C₆ alkyl)-OR¹¹,

—CN, —NR¹¹R¹², —C(═O)R¹¹, —C(═O)OR¹¹, —C(═O)NR¹¹R¹², —CF₃, —OCF₃, —NR¹¹C(═O)R¹², and —NR¹¹C(═O)OR¹², wherein each of said aryl, heterocyclyl and heteroaryl is optionally substituted with 1-4 R⁴³ moieties; and

each R⁴³ is independently selected from the group consisting of halo, alkyl, alkoxy, haloalkyl, cyano, and hydroxyl.

In another embodiment, in Formula (I):

X is N;

ring Y is a 6-membered cycloalkyl which is substituted with an alkyl;

R is selected from the group consisting of H and alkyl-C(═O)—;

R¹ is H;

R³ is selected from the group consisting of H, alkyl, alkenyl, halo, hydroxyl, cyano, H₂NNH—C(═O)—, alkyl-NH—NH—(C═O)—, heteroaryl-NH—NH—C(═O)—, aryl-alkyl-, alkoxy, NH₂-alkyl-, NC-alkyl-, aryl-C(═O)—O-alkyl-, alkyl-O—C(═O)—, H₂N—C(═O)—, aryl-NH—NH—C(═O)—, aryl-NH—C(═O)—, heteroaryl-NH—C(═O)—, alkyl-C(═O)—, alkyl-NH—C(═O)—, aryl-alkyl-NH—C(═O)—, HO-alkyl-aryl-NH—C(═O)—, heteroaryl-alkyl-NH—C(═O)—, heterocyclyl-alkyl-NH—C(═O)—, H₂N-alkyl-NH—C(═O)—, HO-alkyl-NH—C(═O)—, alkyl-O-alkyl-, NC-alkyl-NH—NH—C(═O)—, alkyl-O-alkyl-O-alkyl-, H₂N—C(═S)—NH—N═CH—, alkyl-C(═NOH)—, and heterocyclyl-C(═O)—; wherein each of said alkyl, alkenyl, and the “alkyl” part of aryl-alkyl-, aryl-alkyl-NH—C(═O)— is optionally substituted with 1-2 moieties selected from the group consisting of hydroxy and NH₂; wherein the “aryl” part of each of said aryl-alkyl-, aryl-NH—C(═O)—, and aryl-alkyl-NH—C(═O)— is optionally substituted with 1-2 moieties selected from the group consisting of halo, alkoxy, hydroxyl, NH₂, aryl-C(═O)—NH-and heteroaryl-C(═O)—NH—;

wherein when the “aryl” part of any of said R³ groups contains two adjacent moieties, such moieties have optionally be taken together with the carbon atoms to which they are attached to a form a five to six membered heterocyclyl or heteroaryl; and

R⁴ is selected from the group consisting of H, halo, nitro, H₂N—, alkyl, HO-alkyl-, (HO)₂alkyl-, alkyl-C(═O)-alkyl-C(═O)—NH—, alkenyl-C(═O)-alkyl-C(═O)—NH—, H₂N—C(═O)-alkyl-whose “alkyl” is optionally substituted with an alkyl-C(═O)—NH—, NC-alkyl-, H₂N-alkyl-, alkyl-O—C(═O)—NH—, HO—C(═O)—NH—, alkyl-C(═O)—O-alkyl-C(═O)—NH—, alkyl-O—C(═O)-alkenyl-, heteroaryl-C(═O)—NH—, heterocyclyl, HO-alkynyl-, alkyl-O-alkyl-NH—, HO-alkyl-NH—, alkyl-S(═O)₂NH—, alkyl-O—C(═O)—, HO-alkyl-NH—C(═O)—, (HO)₂alkyl-NH—C(═O)—, H₂N-alkyl-NH—C(═O)—, heterocyclyl-alkyl-NH—C(═O)—, heteroaryl-alkyl-NH—C(═O)—, alkenyl-NH—C(═O)—, H₂N—NH—C(═O)—, H₂N—C(═O)—, alkyl-C(═O)—NH—, heteroaryl-C(═O)—, aryl-NH—C(═O)—, heteroaryl-NH—C(═O)—, and aryl that is optionally substituted with 1-2 moieties selected from the group consisting of hydroxy, alkoxy, haloalkoxy, cyano, H₂N—, alkyl-S, alkyl-S(═O)—, and alkyl-S(═O)₂—.

In another embodiment, the compound of Formula (I) is selected from the group consisting of compounds listed in the table below, or a pharmaceutically acceptable salt, solvate, or ester hereof. This table also lists KSP inhibitory activities (IC₅₀ rating) based on end-point assay. IC₅₀ values greater than 10000 nM (i.e., >10 μM) are designated as D class. IC₅₀ values between 1000 nM (1 μM) and 10000 nM (10 μM) are designated as C class. IC₅₀ values between 100 nM (0.1 μM) and less than 1000 nM (<1 μM) are designated as B class. IC₅₀ values less than 100 nM (<0.1 μM) are designated as A class. The syntheis and characterization of these compounds is described hereinbelow in the “EXAMPLES” section of the present application.

TABLE 1

| | | IC₅₀ |
|---|---|---|
| Example | Structure | Rating |
| 1 | 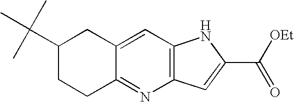 | C |
| 2 | 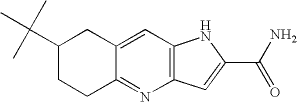 | A |
| 3 | 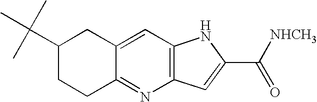 | A |
| 4 | 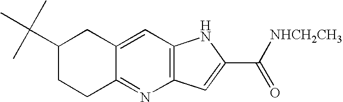 | B |
| 5 | 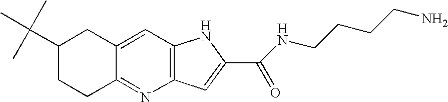 | B |
| 6 | 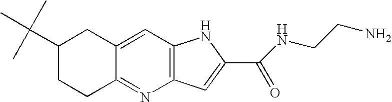 | B |
| 7 | 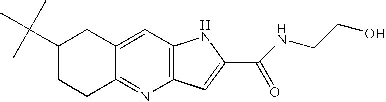 | B |
| 8 | 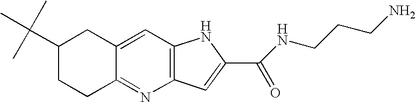 | C |
| 9 | 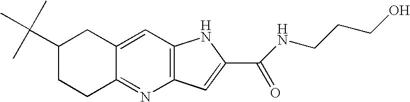 | B |
| 10 | 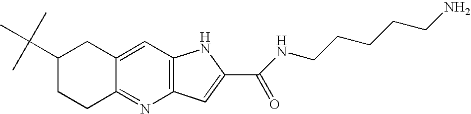 | B |
| 11 | 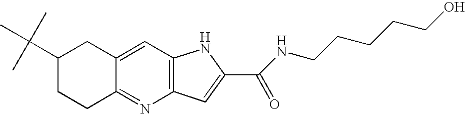 | C |
| 12 | 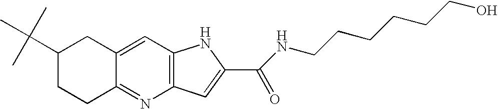 | D |
| 13 | 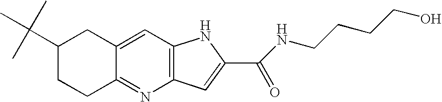 | C |
| 14 | 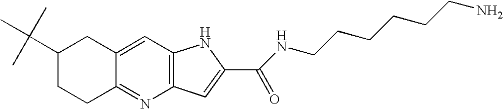 | B |
| 15 | 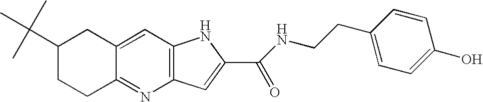 | D |
| 16 | 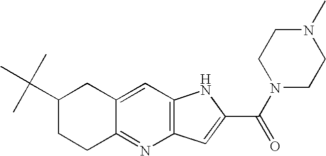 | D |
| 17 | 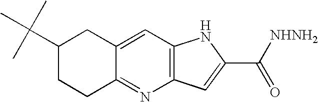 | A |
| 18 | 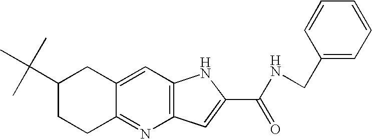 | D |
| 19 | 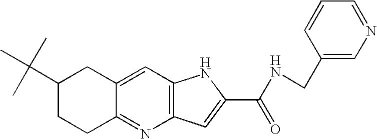 | D |
| 20 | 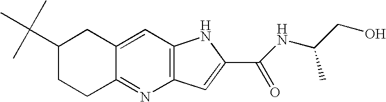 | C |
| 21A | 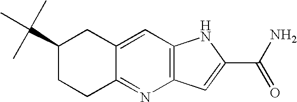 | A |
| 21B | 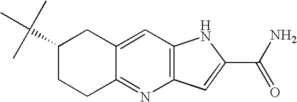 | B |
| 22A | 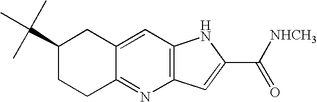 | A |
| 22B | 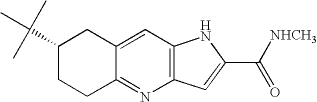 | B |
| 23 | 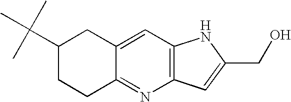 | A |
| 24 | 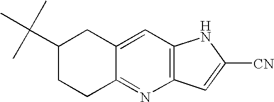 | A |
| 25 | 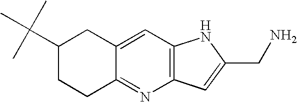 | B |
| 26 | 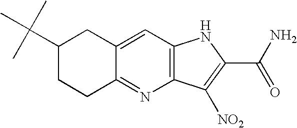 | D |
| 27 | 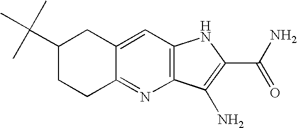 | B |
| 28 | 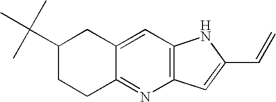 | C |
| 29 | 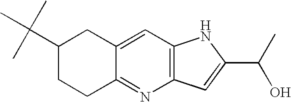 | B |
| 30 | 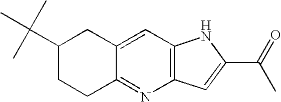 | B |
| 31 | 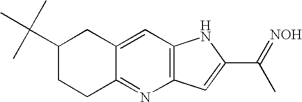 | D |
| 32 | 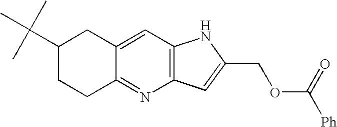 | D |
| 33 | 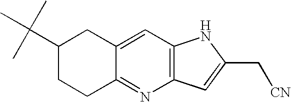 | C |
| 34 | 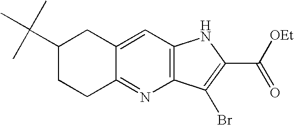 | D |
| 35 | 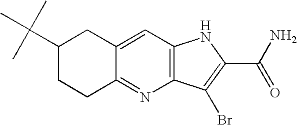 | D |
| 36 | 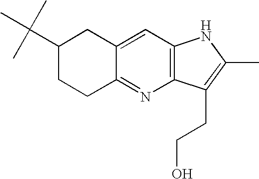 | D |
| 37 | 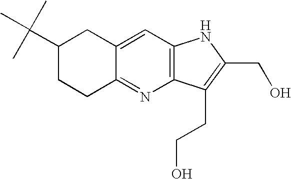 | D |
| 38 | 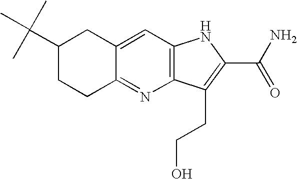 | C |
| 39 | 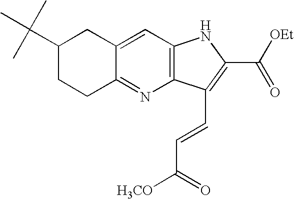 | D |
| 40 | 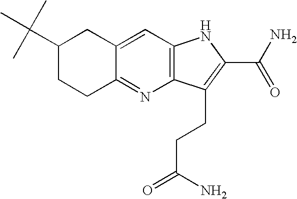 | C |
| 41 | 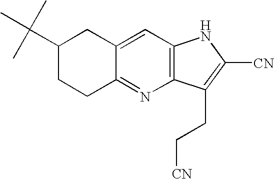 | C |
| 42 | 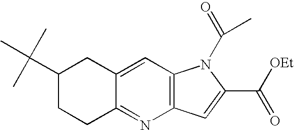 | D |
| 43 | 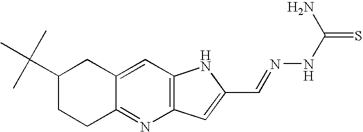 | D |
| 44 | 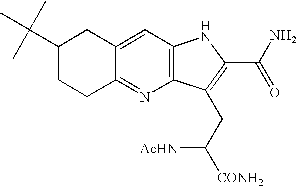 | D |
| 45 | 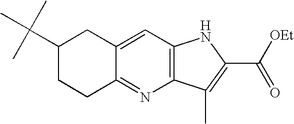 | D |
| 46 | 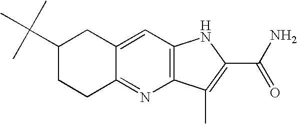 | D |
| 47 | 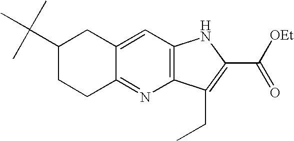 | D |
| 48 | 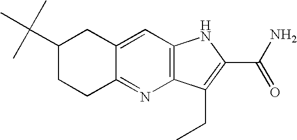 | C |
| 49 | 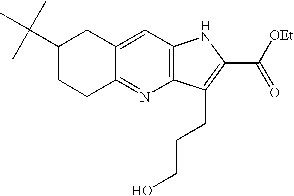 | D |
| 50 | 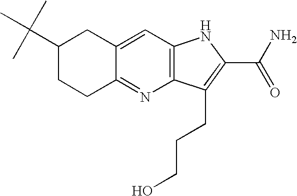 | B |
| 51 | 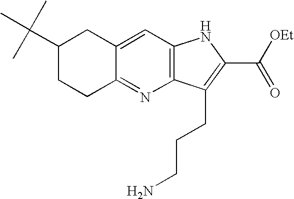 | D |
| 52 | 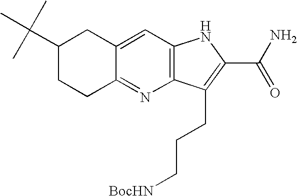 | D |
| 53 | 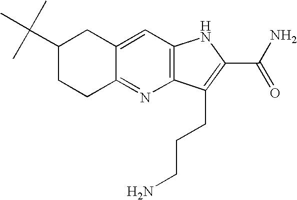 | D |
| 54 | 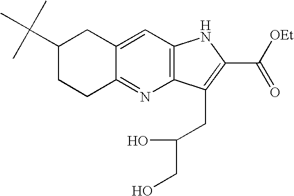 | D |
| 55 | 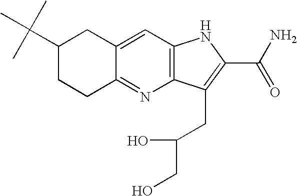 | D |
| 56 | 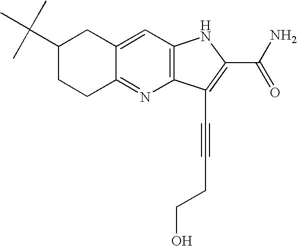 | D |
| 57 | 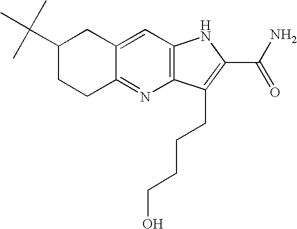 | B |
| 58 | 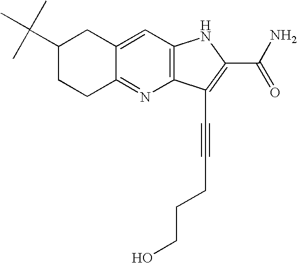 | D |
| 59 | 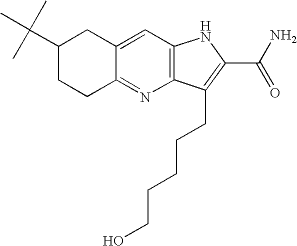 | C |
| 60 | 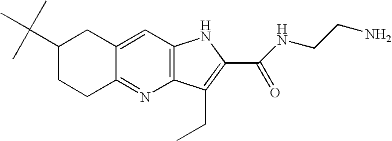 | >B |
| 61 | 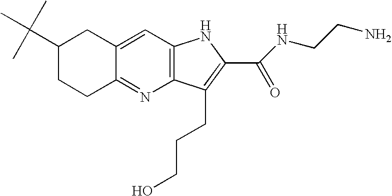 | >B |
| 62 | 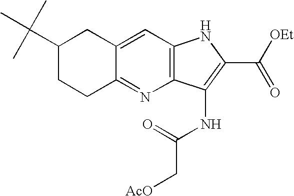 | D |
| 63 | 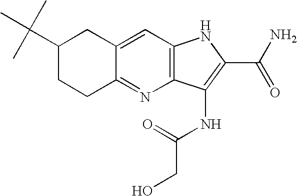 | C |
| 64 | 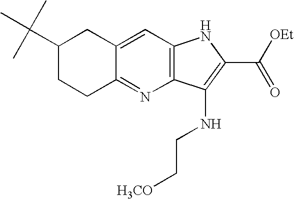 | D |
| 65 | 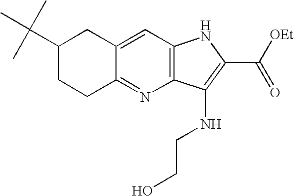 | D |
| 66 | 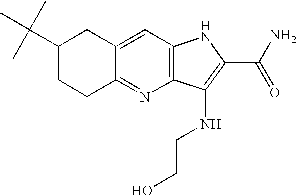 | C |
| 67 | 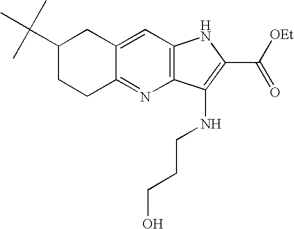 | D |
| 68 | 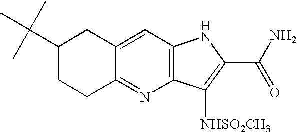 | C |
| 69 | 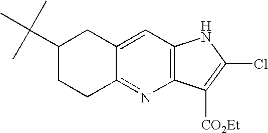 | D |
| 70 | 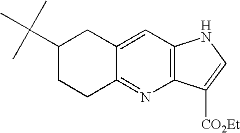 | D |
| 71 | 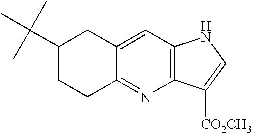 | D |
| 72 | 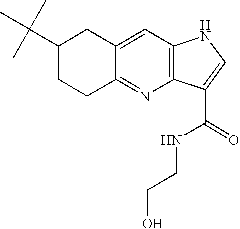 | C |
| 73 | 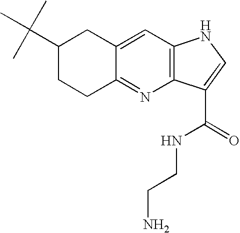 | D |
| 74 | 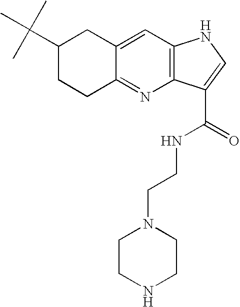 | D |
| 75 | 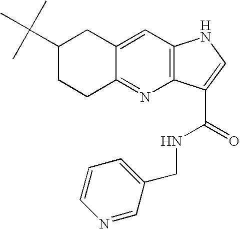 | D |
| 76 | 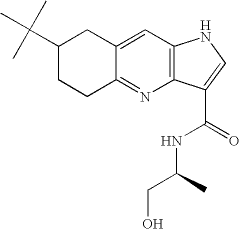 | D |
| 77 | 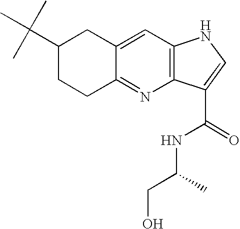 | D |
| 78 | 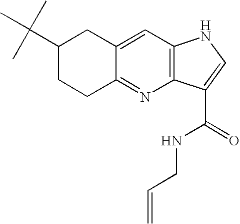 | D |
| 79 | 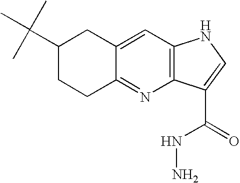 | B |
| 80 | 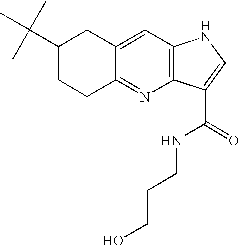 | C |
| 81 | 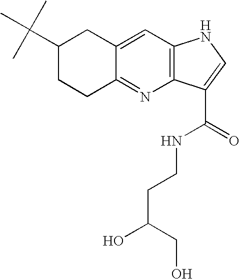 | D |
| 82 | 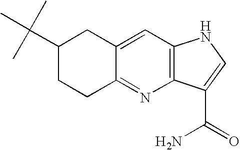 | B |
| 83 | 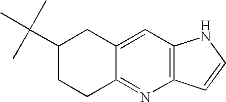 | A |
| 84 | 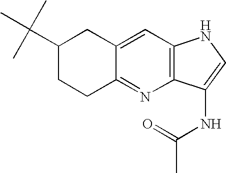 | C |
| 85 | 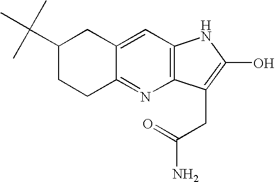 | D |
| 86 | 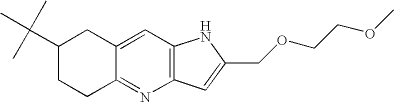 | B |
| 87 | 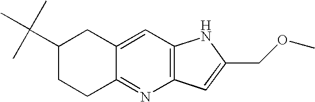 | B |
| 88 | 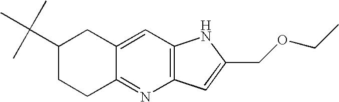 | B |
| 89 | 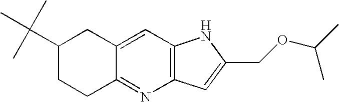 | D |
| 90 | 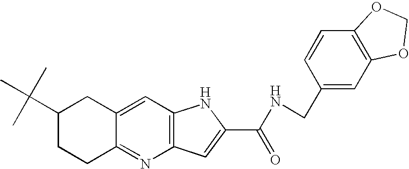 | D |
| 91 | 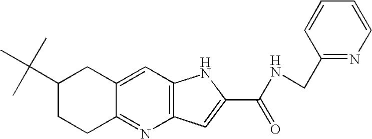 | D |
| 92 | 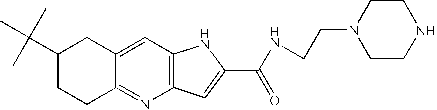 | C |
| 93 | 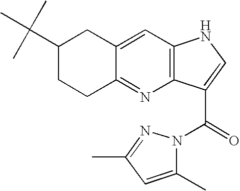 | D |
| 94 | 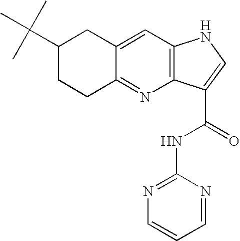 | D |
| 95 | 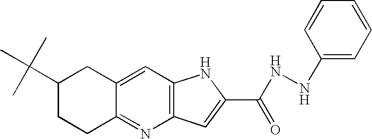 | D |
| 96 | 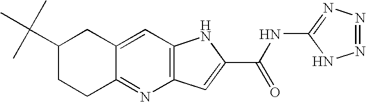 | B |
| 97 | 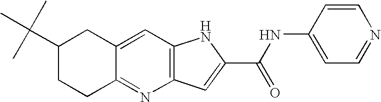 | B |
| 98 | 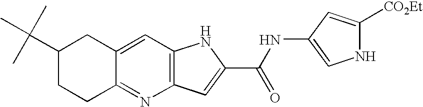 | D |
| 99 | 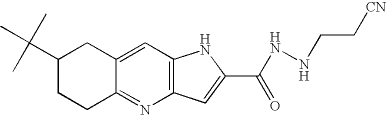 | D |
| 100 | 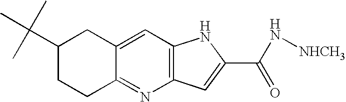 | D |
| 101 | 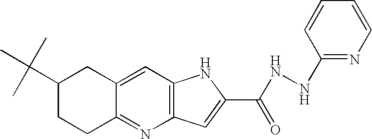 | B |
| 102 | 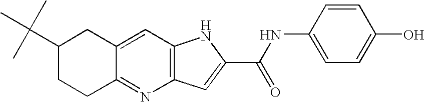 | B |
| 103A | 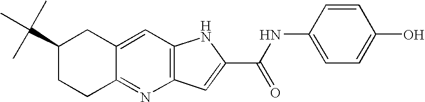 | B |
| 103B | 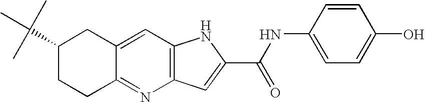 | D |
| 104 | 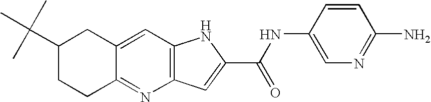 | B |
| 105 | 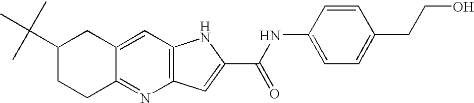 | C |
| 106 | 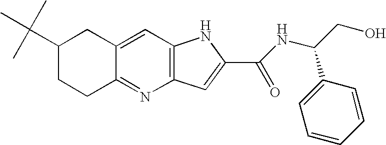 | B |
| 107 | 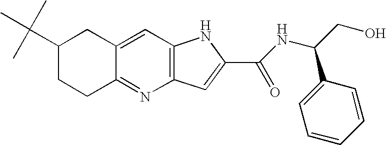 | D |
| 108 | 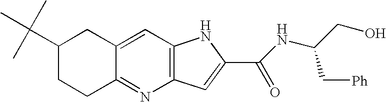 | D |
| 109 | 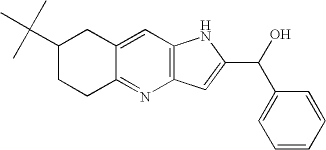 | D |
| 110 | 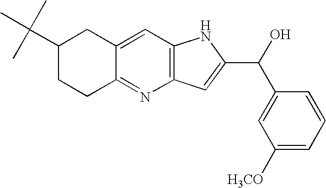 | D |
| 111 | 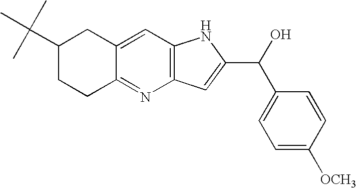 | D |
| 112 | 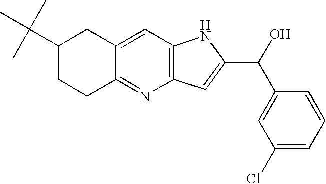 | D |
| 113 | 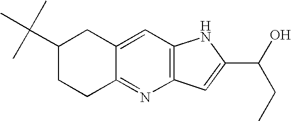 | B |
| 114 | 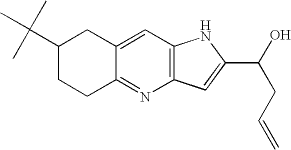 | B |
| 115 | 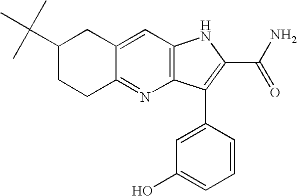 | C |
| 116 | 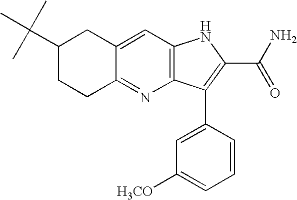 | D |
| 117 | 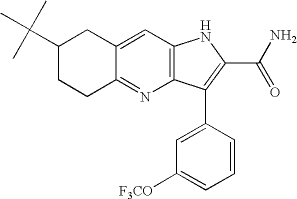 | D |
| 118 | 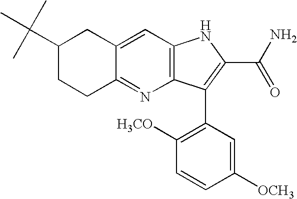 | D |
| 119 | 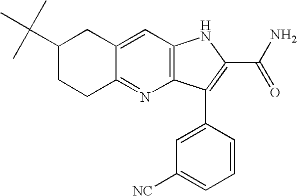 | D |
| 120 | 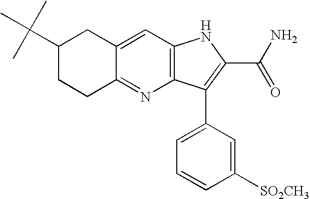 | B |
| 121 | 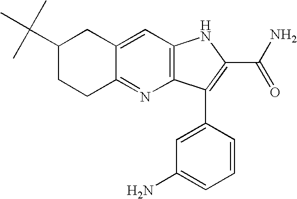 | D |
| 121 | 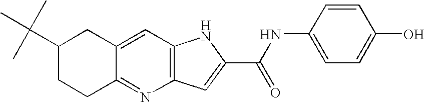 | B |
| 122 | 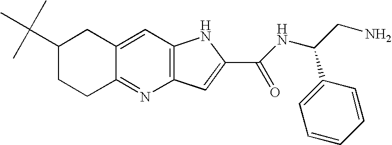 | B |
| 123 | 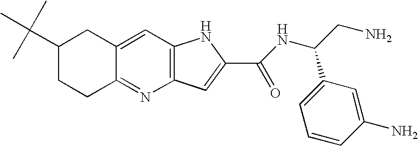 | A |
| 124 | 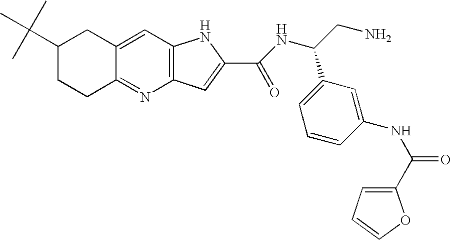 | A |

In another embodiment, the compound of Formula (I) is selected from the group consisting of:

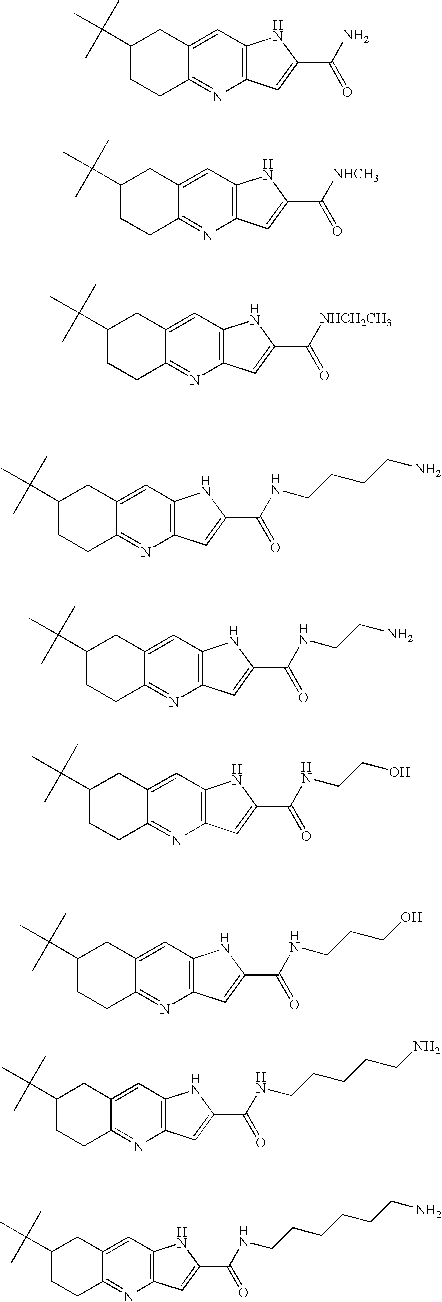

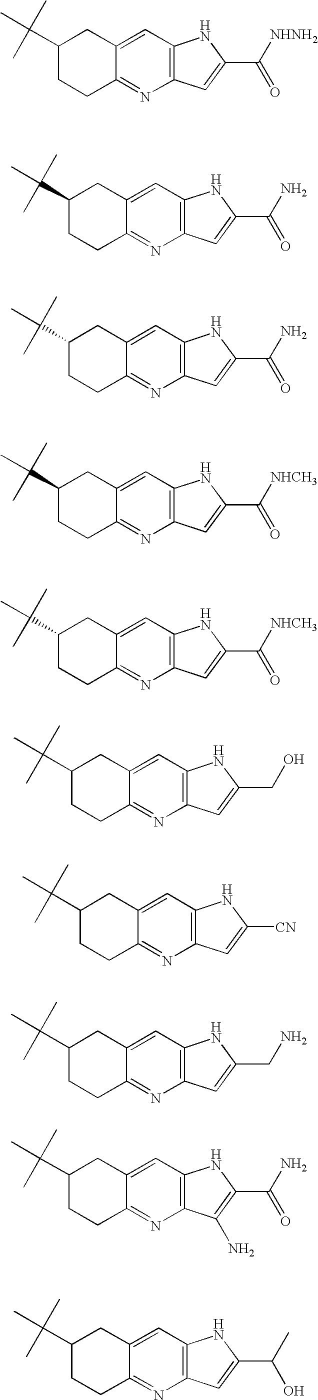

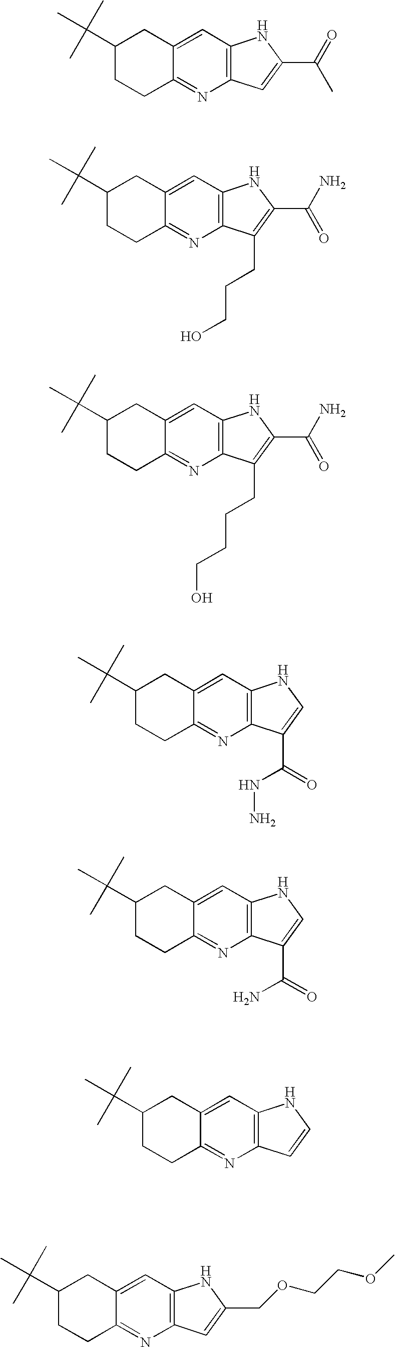

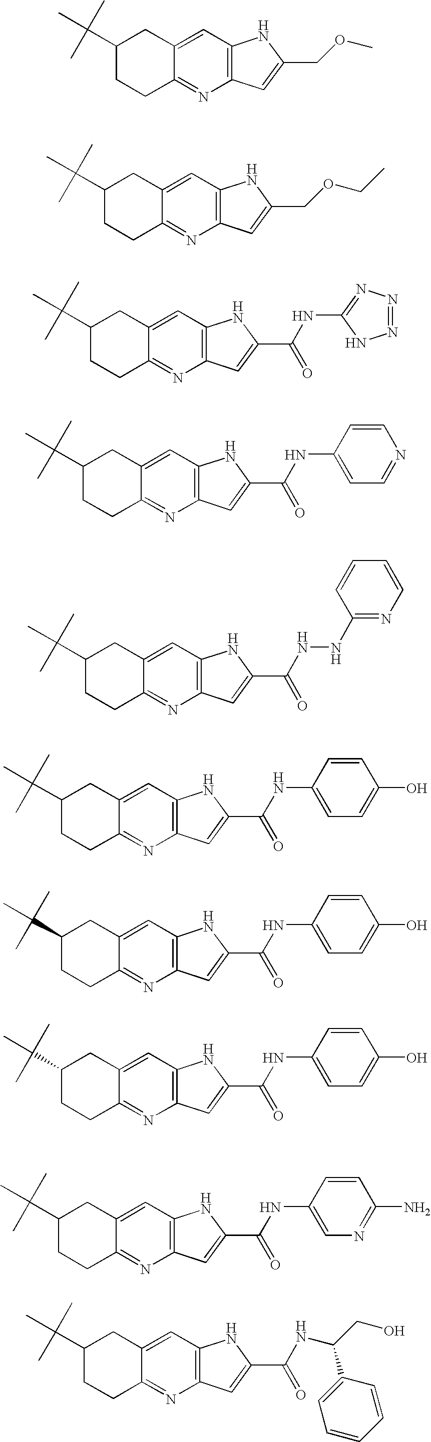

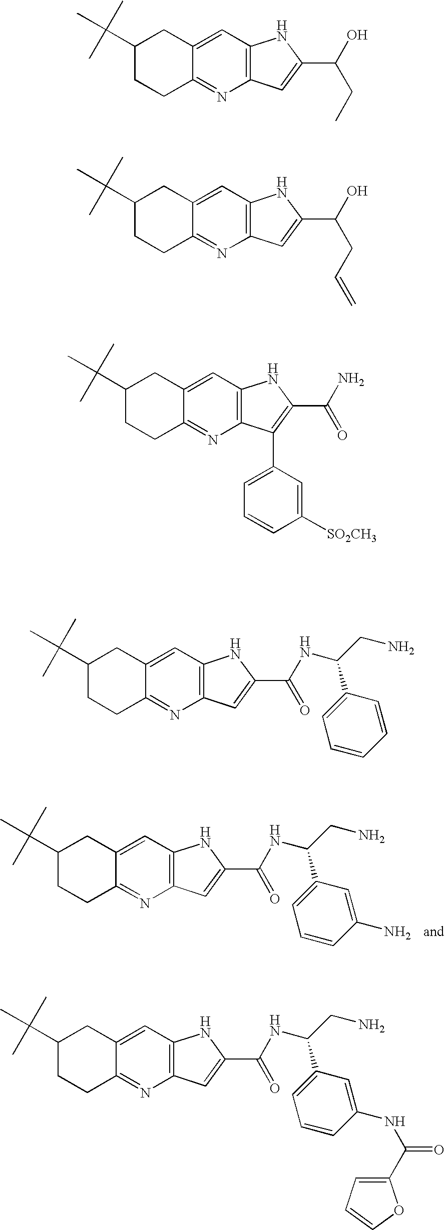

or a pharmaceutically acceptable salt, solvate or ester thereof.

In other embodiments, the present invention provides processes for producing such compounds, pharmaceutical formulations or compositions comprising one or more of such compounds, and methods of treating or preventing one or more conditions or diseases associated with KSP kinesin activity such as those discussed in detail below.

As used above, and throughout the specification, the following terms, unless otherwise indicated, shall be understood to have the following meanings:

“Subject” includes both mammals and non-mammalian animals.

“Mammal” includes humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. It should be noted that any atom with unsatisfied valences in the text, schemes, examples and tables herein is assumed to have the hydrogen atom(s) to satisfy the valences.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, etc.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl. “Alkyl” includes “Alkylene” which refers to a difunctional group obtained by removal of a hydrogen atom from an alkyl group that is defined above. Non-limiting examples of alkylene include methylene (—CH₂—), ethylene (—CH₂CH₂—) and propylene (—C₃H₆—; which may be linear or branched).

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

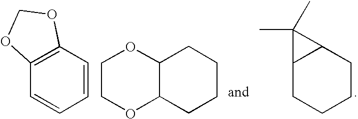

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

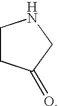

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, dihydroimidazole, dihydrooxazole, dihydrooxadiazole, dihydrothiazole, 3,4-dihydro-2H-pyran, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

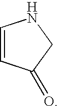

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

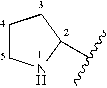

there is no —OH attached directly to carbons marked 2 and 5.

It should also be noted that tautomeric forms such as, for example, the moieties:

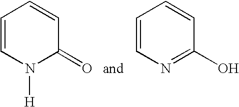

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkylsilyl” means an alkyl-Si— group in which alkyl is as previously defined and the point of attachment to the parent moiety is on Si. Preferred alkylsilyls contain lower alkyl. An example of an alkylsilyl group is trimethylsilyl (—Si(CH₃)₃).

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-(C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1)1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula (I), and salts, solvates, esters and prodrugs thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula (I), and of the salts, solvates, esters and prodrugs of the compounds of Formula (I), are intended to be included in the present invention.

Generally, the compounds of Formula (I) can be prepared by a variety of methods well known to those skilled in the art, for example, by the methods as outlined in Scheme 1 below and in the examples disclosed herein:

The compounds of the invention can be useful in a variety of applications involving alteration of mitosis. As will be appreciated by those skilled in the art, mitosis may be altered in a variety of ways; that is, one can affect mitosis either by increasing or decreasing the activity of a component in the mitotic pathway. Mitosis may be affected (e.g., disrupted) by disturbing equilibrium, either by inhibiting or activating certain components. Similar approaches may be used to alter meiosis.

In a particular embodiment, the compounds of the invention can be used to inhibit mitotic spindle formation, thus causing prolonged cell cycle arrest in mitosis. By “inhibit” in this context is meant decreasing or interfering with mitotic spindle formation or causing mitotic spindle dysfunction. By “mitotic spindle formation” herein is meant organization of microtubules into bipolar structures by mitotic kinesins. By “mitotic spindle dysfunction” herein is meant mitotic arrest and monopolar spindle formation.

The compounds of the invention can be useful for binding to, and/or inhibiting the activity of, a mitotic kinesin, KSP. In one embodiment, the KSP is human KSP, although the compounds may be used to bind to or inhibit the activity of KSP kinesins from other organisms. In this context, “inhibit” means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP (see U.S. Pat. No. 6,437,115). In addition, the present compounds are also useful for binding to or modulating other mitotic kinesins.

The compounds of the invention can be used to treat cellular proliferation diseases. Such disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or subjects afflicted or subject to impending affliction with any one of these disorders or states.

The compounds, compositions and methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-A-phenyl-2,2-dimethylpropanoate, 4,4′-dihydroxybenzophenone-2,4-dinitrophenyl-ydrazone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl) retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycaminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine, and mixture thereof.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino) ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kif14, inhibitors of Mphosph1 and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichloropheny)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin(ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401 (1999).

Examples of farnesyl protein transferase inhibitors include SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-1-piperidinecarboxamide from Schering-Plough Corporation, Kenilworth, N.J.), tipifarnib (Zarnestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α (for example Intron and Peg-Intron), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). Examples of TAFIa inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELIEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the α_{c}β₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, α₁β₁, α₂β₁, α₅β₁, α₆β₁ and α₆β₄ integrins.

Some examples of tyrosine kinase inhibitors include N-(trifluoromethylphenyl)-5-methylisoxazol-4-carboxamide, 3-[(2,4-dimethylpyrrol-5-yl)methylidenyl)indolin-2-one,17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Opthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy)phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux and mixtures thereof.

Another embodiment of the present invention is the use of the present compounds in combination with gene therapy for the treatment of cancer. For an overview of genetic strategies to treating cancer, see Hall et al (Am J Hum Genet 61:785-789, 1997) and Kufe et al (Cancer Medicine, 5th Ed, pp 876-889, B C Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The present compounds can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drug, an inhibitor of cell proliferation and survival signaling, an agent that interfers with a cell cycle checkpoint, and an apoptosis inducing agent.

In one embodiment, the present invention empassesses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention is a method of treating cancer comprising administering a therapeutically effective amount of at least one compound of Formula (I) in combination with radiation therapy and at least one compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interfers with a cell cycle checkpoint, and an apoptosis inducing agent.

Yet another embodiment of the invention is a method of treating cancer comprising administering a therapeutically effective amount of at least one compound of Formula (I) in combination with paclitaxel or trastuzumab.

The present invention also includes a pharmaceutical composition useful for treating or preventing cellular proliferation diseases (such as cancer, hyperplasia, cardiac hypertrophy, autoimmune diseases, fungal disorders, arthritis, graft rejection, inflammatory bowel disease, immune disorders, inflammation, and cellular proliferation induced after medical procedures) that comprises a therapeutically effective amount of at least one compound of Formula (I) and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interfers with a cell cycle checkpoint, and an apoptosis inducing agent.

Another aspect of this invention relates to a method of selectively inhibiting KSP kinesin activity in a subject (such as a cell, animal or human) in need thereof, comprising contacting said subject with at least one compound of Formula (I) or a pharmaceutically acceptable salt or ester thereof.

Preferred KSP kinesin inhibitors are those which can specifically inhibit KSP kinesin activity at low concentrations, for example, those that cause a level of inhibition of 50% or greater at a concentration of 50 μM or less, more preferably 100 nM or less, most preferably 50 nM or less.

Another aspect of this invention relates to a method of treating or preventing a disease or condition associated with KSP in a subject (e.g., human) in need thereof comprising administering a therapeutically effective amount of at least one compound of Formula (I) or a pharmaceutically acceptable salt or ester thereof to said subject.

A preferred dosage is about 0.001 to 500 mg/kg of body weight/day of a compound of Formula (I) or a pharmaceutically acceptable salt or ester thereof. An especially preferred dosage is about 0.01 to 25 mg/kg of body weight/day of a compound of Formula (I) or a pharmaceutically acceptable salt or ester thereof.

The phrases “effective amount” and “therapeutically effective amount” mean that amount of a compound of Formula (I), and other pharmacological or therapeutic agents described herein, that will elicit a biological or medical response of a tissue, a system, or a subject (e.g., animal or human) that is being sought by the administrator (such as a researcher, doctor or veterinarian) which includes alleviation of the symptoms of the condition or disease being treated and the prevention, slowing or halting of progression of one or more cellular proliferation diseases. The formulations or compositions, combinations and treatments of the present invention can be administered by any suitable means which produce contact of these compounds with the site of action in the body of, for example, a mammal or human.

For administration of pharmaceutically acceptable salts of the above compounds, the weights indicated above refer to the weight of the acid equivalent or the base equivalent of the therapeutic compound derived from the salt.

As described above, this invention includes combinations comprising an amount of at least one compound of Formula (I) or a pharmaceutically acceptable salt or ester thereof, and an amount of one or more additional therapeutic agents listed above (administered together or sequentially) wherein the amounts of the compounds/treatments result in desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of Formula (I) and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

If formulated as a fixed dose, such combination products employ the compounds of this invention within the dosage range described herein and the other pharmaceutically active agent or treatment within its dosage range. Compounds of Formula (I) may also be administered sequentially with known therapeutic agents when a combination formulation is inappropriate. The invention is not limited in the sequence of administration; compounds of Formula (I) may be administered either prior to or after administration of the known therapeutic agent. Such techniques are within the skills of persons skilled in the art as well as attending physicians.

The pharmacological properties of the compounds of this invention may be confirmed by a number of pharmacological assays. The inhibitory activity of the present compounds towards KSP may be assayed by methods known in the art, for example, by using the methods as described in the examples.

While it is possible for the active ingredient to be administered alone, it is preferable to present it as a pharmaceutical composition. The compositions of the present invention comprise at least one active ingredient, as defined above, together with one or more acceptable carriers, adjuvants or vehicles thereof and optionally other therapeutic agents. Each carrier, adjuvant or vehicle must be acceptable in the sense of being compatible with the other ingredients of the composition and not injurious to the mammal in need of treatment.

Accordingly, this invention also relates to pharmaceutical compositions comprising at least one compound of Formula (I), or a pharmaceutically acceptable salt or ester thereof and at least one pharmaceutically acceptable carrier, adjuvant or vehicle.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts or esters thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I) or a pharmaceutically acceptable salt or ester thereof and at least one pharmaceutically acceptable carrier, adjuvant or vehicle.

Yet another aspect of this invention is a kit comprising an amount of at least one compound of Formula (I) or a pharmaceutically acceptable salt or ester thereof and an amount of at least one additional therapeutic agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art.

The following solvents and reagents may be referred to by their abbreviations in parenthesis:

Thin layer chromatography: TLC
dichloromethane: CH₂Cl₂
ethyl acetate: AcOEt or EtOAc
methanol: MeOH
trifluoroacetate: TFA
triethylamine: Et₃N or TEA
butoxycarbonyl: n-Boc or Boc
nuclear magnetic resonance spectroscopy: NMR
liquid chromatography mass spectrometry: LCMS
high resolution mass spectrometry: HRMS
milliliters: mL
millimoles: mmol
microliters: μl
grams: g
milligrams: mg
room temperature or rt (ambient): about 25° C.
dimethoxyethane: DME

### EXAMPLES

### General Methods of Preparation

Compounds of the present invention can be prepared by a number of methods evident to one skilled in the art. Preferred methods include, but are not limited to, the general synthetic procedures described herein. One skilled in the art will recognize that one route will be optimal depending upon the choice of appendage substituents. Additionally, one skilled in the art will recognize that in some cases the order of steps may be varied to avoid functional group incompatibilities. One skilled in the art will also recognize that modifications of the R³ and R⁴ groups by the methods known to one skilled in the art can provide compounds with different R³ and R⁴ groups.

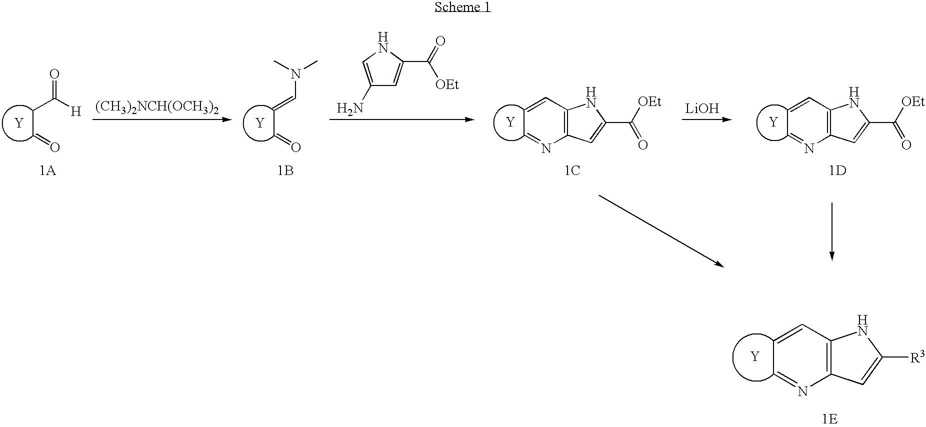

The appropriately substituted pyrrole derivatives of Formula (I) can be prepared as follows. The ketone 1A was treated with N,N-dimethylformamide dimethyl acetal to provide 1B which was cyclized with 4-amino-1H-pyrrole-2-carboxylic acid ethyl ester to afford the compound 1C. The ester can be hydrolyzed to carboxylic acid 1D under basic conditions. The ester 1C or acid 1D can be converted to various R³ group by methods known to one skilled in the art such as reduction, treatment with a nucleophile or with some standard modifications. For example, reaction of the ester 1C with appropriately substituted or unsubstituted amine in absence or presence of sodium cyanide can afford the amide products. Alternatively, the amides can be prepared by the treatment of appropriate amine with the reactive carboxy derivative (e.g. acid chloride) of acid 1D or reaction with acid 1D in presence of suitable coupling reagent (e.g. HATU).

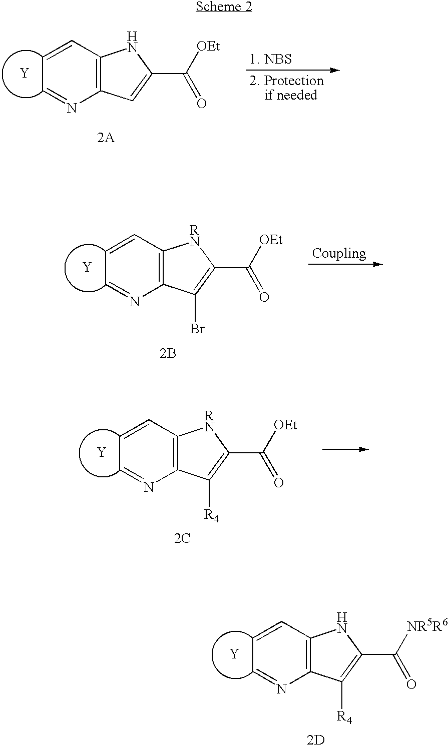

Some of the R⁴ substituted compounds of Formula (I) where a cabon or nitrogen is directly attached to the pyrrole ring can be prepared as follows. Treatment of pyrrole derivative 2A with a brominating reagent preferably N-bromosuccinimide in a suitable solvent provided compound 2B. The pyrrole NH group can be protected with a suitable protecting group preferably Boc if necessary for the next reaction. The bromo compound 2B was reacted with appropriate boronic acids, tin reagents or alkynes to provide carbon linked derivatives 2C whereas treatment of 2B with an amine under Buckwald type coupling conditions can afford nitrogen linked derivatives 2C. Deprotection of the protecting group if needed followed by treatment with appropriate amines provided compounds 2D. Some of the R⁴ groups can be modified at the appropriate stage by the methods known to one skilled in the art.

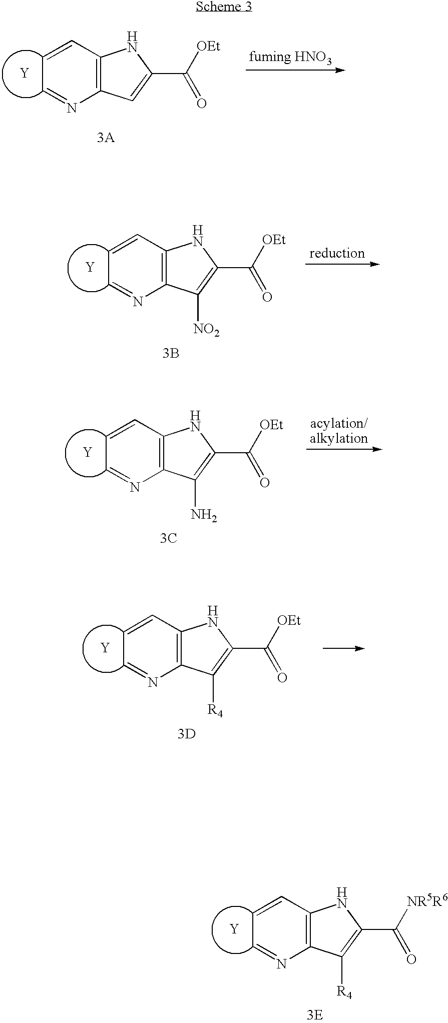

Some of the R⁴ substituted compounds of Formula I where nitrogen is attached to the pyrrole ring can also be prepared as follows. Treatment of pyrrole derivative 3A with a nitrating reagent preferably fuming nitric acid provided compound 3B. The reduction of nitro group afforded the amino compound 3C which could be acylated or alkylated by the methods known to one skilled in the art to provide compounds with different R⁴ groups. Further treatment of compound 3D with amines by following suitable method as described in the Scheme 1 can afford compounds 3E. Some of the R⁴ groups can be modified at the appropriate stage by the methods known to one skilled in the art.

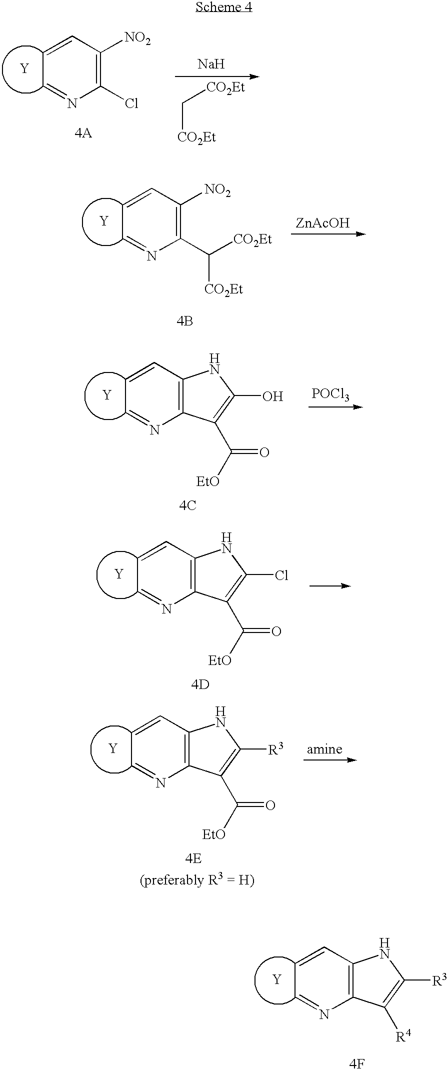

Some of the R³ and R⁴ substituted compounds of Formula I where preferably R⁴ is an amide group can be prepared as follows. Treatment of compound 4A with diethyl malonate in presence of a base afforded the compound 4B. Reduction of the nitro group preferably with zinc/acetic acid followed by treatment with phosphorus oxychloride provided the compound 4D. The chloro group of compound 4D could be functionalized to afford different R⁴ groups by the methods known to one skilled in the art or preferably it can be reduced to provide compound 4E where preferably R⁴ is H. The ester group of the compound 4E could be treated with different amines by the methods as described in the scheme 1 preferably reaction with amines in presence of sodium cyanide to provide compounds 4F where R⁴ is an amide group.

Illustrating the invention are the following examples which, however, are not to be considered as limiting the invention to their details. Unless otherwise indicated, all parts and percentages in the following examples, as well as throughout the specification, are by weight.

### Specific Methods of Preparation—Examples

### Example 1

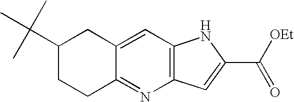

### Preparative Example 1

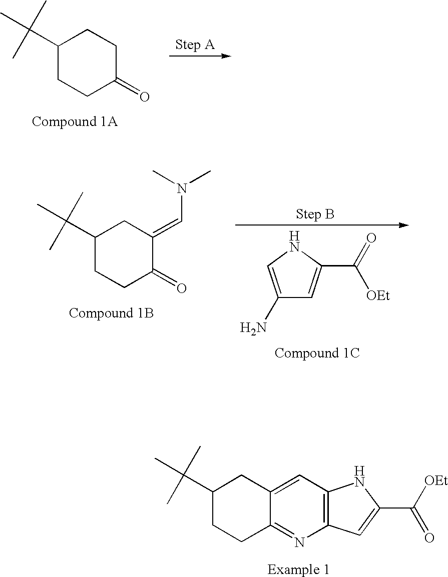

### Step A:

A mixture of 4-tert-butylcyclohexanone (10 g, 64.83 mmol, 1 equiv), N,N-dimethylformamide dimethyl acetal (8.6 mL, 64.83 mmol, 1 equiv) and toluene (20 mL) was heated at 100 for 18 hours. Concentrated to give the 13.5 g of the compound 1B which was used in the next reaction without further purification.

### Step B:

A mixture of 4-nitropyrrole-2-carboxylic acid ethyl ester, EtOH, 10% Pd(OH)₂—C was stirred under H2 parr shaker at 40 psi for 18 hours. Filtered over celite and washed with ethanol. The filterate was concentrated to afford the compound 1C. A mixture of compound 1B (5 g, 23.9 mmol, 1 equiv), compound 1C (3.68 g, 23.9 mmol, 1 equiv) and acetic acid (100 mL) was heated at 80° C. for 72 hours. Cooled to room temperature and concentrated. To the residue was added CH₂Cl₂ (500 mL) and washed with sat. NaHCO₃ (3×300 mL). The organic layer was dried over NaSO₄, filtered and concentrated. To the residue was added CH₂Cl₂ (500 mL) followed by diethyl ether (200 mL) and filtered the resulting solid. The solid was washed with diethyl ether and dried to give Example 1 (2.5 g). LCMS: MH⁺=301.

### Example 2

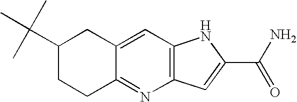

### Preparative Example 2

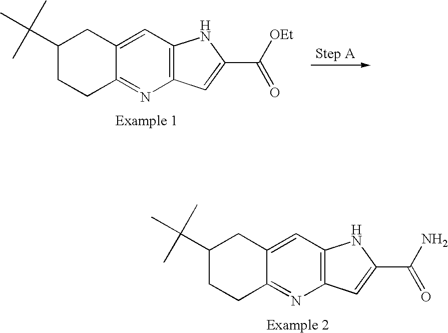

### Step A:

A mixture of Example 1 (0.02 g, 0.066 mmol, 1 equiv) and methanol (10 mL) was saturated ammonia and heated at 60° C. for 72 hours. Concentrated and purified by flash chromatography eluting with 7% MeOH/EtOAc to give Example 2 (10 mg). LCMS: MH⁺=272.

### Example 3

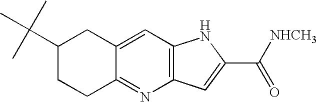

### Preparative Example 3

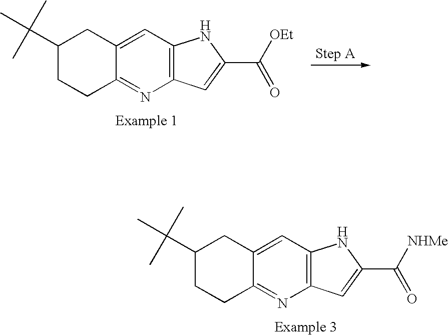

### Step A:

A mixture of Example 1 (0.03 g, 0.1 mmol, 1 equiv) and a 2M solution of methylamine in methanol (5 mL) was heated at 64° C. for 18 hours. Concentrated and purified by flash chromatography eluting with 9% MeOH/EtOAc to give Example 3 (25 mg). LCMS: MH⁺=286.

### Example 4

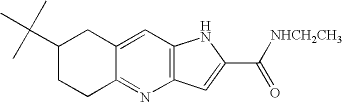

Following a procedure similar to that of Example 3, but using the ethylamine in place of methylamine the title compound Example 4 was prepared. LCMS: MH⁺=300.

### Example 5

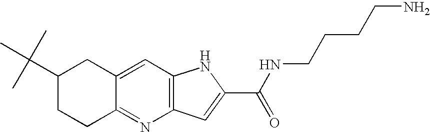

### Preparative Example 5

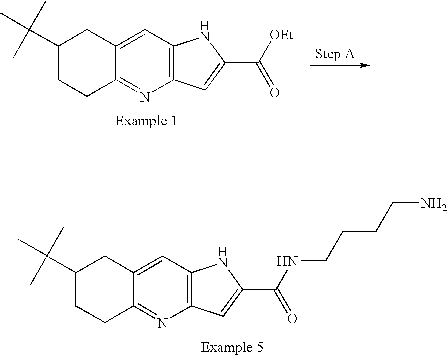

### Step A:

A mixture of Example 1 (0.03 g, 0.1 mmol, 1 equiv) and 1,4-diaminobutane (4 mL) was at 120° C. for 18 hours. Poured into CH₂Cl₂ (200 mL) and washed with water (100 mL). The organic layer was dried over Na₂SO₄, filtered and concentrated. Purified by flash chromatography eluting with 15% MeOH(NH₃)/CH₂Cl₂ to give the title compound Example 5 (40 mg). LCMS: MH⁺=343.

### Examples 6-12

Following a procedure similar to that of Example 5, but using the appropriately substituted amine, the compounds in Table 1 were prepared from Example 1.

| Example | Structure | LCMS: MH⁺ |
|---|---|---|
| 6 | 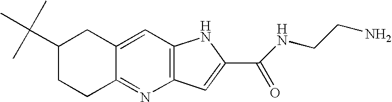 | 315 |
| 7 | 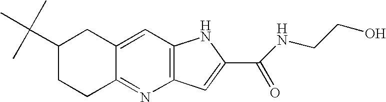 | 316 |
| 8 | 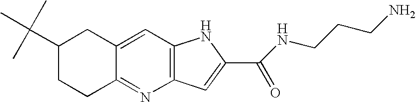 | 329 |
| 9 | 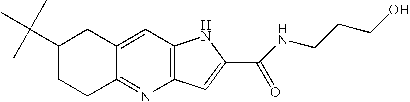 | 330 |
| 10 | 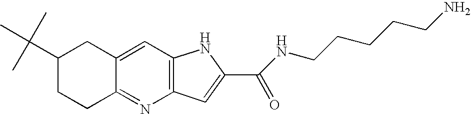 | 357 |
| 11 | 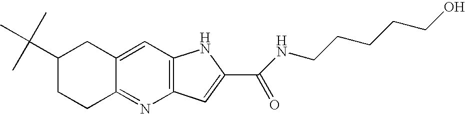 | 358 |
| 12 | 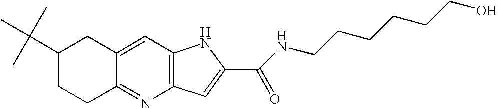 | 372 |
| 13 | 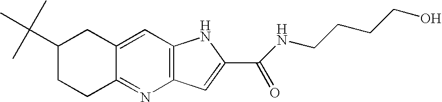 | 344 |
| 14 | 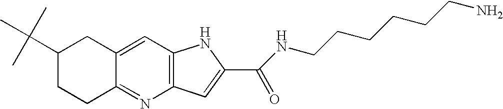 | 371 |
| 15 | 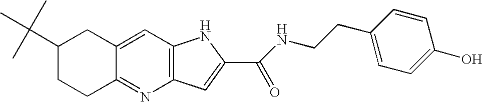 | 392 |
| 16 | 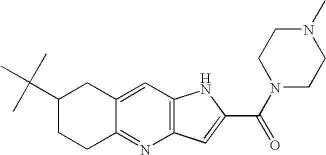 | 355 |
| 17 | 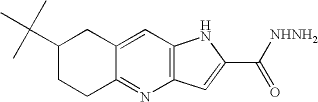 | 287 |
| 18 | 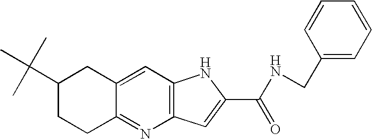 | 362 |
| 19 | 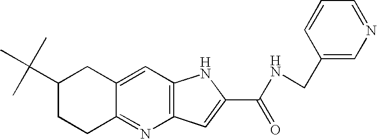 | 363 |

### Example 20

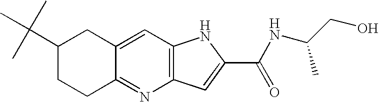

### Preparative Example 20

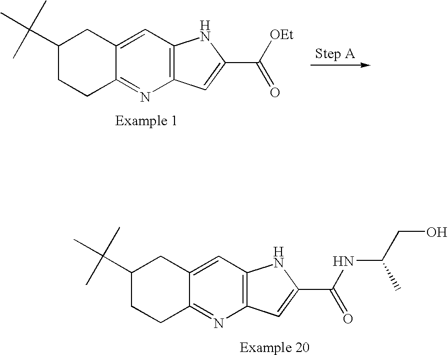

### Step A:

A mixture of Example 1 (0.03 g, 0.1 mmol, 1 equiv), (S)-2-amino-1-propanol (0.078 mL, 1 mmol, 10 equiv), sodium cyanide (0.005 g, 0.1 mmol, 1 equiv) and o-xylene was heated at 138° C. for 18 hours. Cooled to room temperature, diluted with EtOAC (200 mL) and washed with water (100 mL). The organic layer was dried over Na₂SO₄, filtered and concentrated. Purified by flash chromatography eluting with 100% EtOAc and 5% MeOH(NH₃)/EtOAc to give the title compound Example 20 (10 mg). LCMS: MH⁺=330.

### Examples 21A and 21B

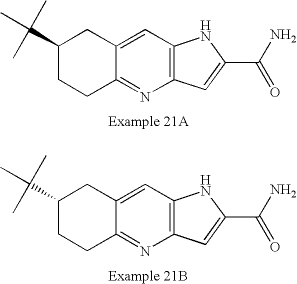

### Preparative Examples 21A and 21B

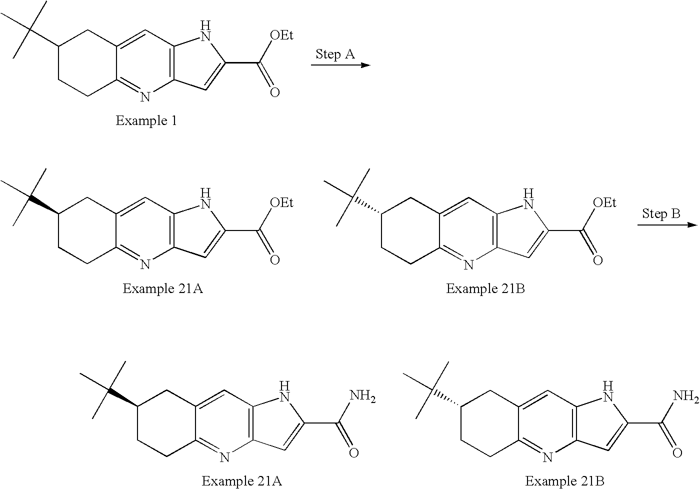

### Step A:

Example 1 (0.25 g) was separated on HPLC using Chiralpak AD column eluting with 1/1/IPA/hexane. Isomer A, compound 21A (0.082 g), and isomer B, compound 21B (0.11 g) were obtained.

### Step B:

Compounds 21A and 21B were converted to Example 21A and Example 21B respectively using the procedure as described for the preparation of Example 2 from Example 1. Example 21A, LCMS: MH⁺=272 and Example 21B, LCMS: MH⁺=272.

### Examples 22A and 22B

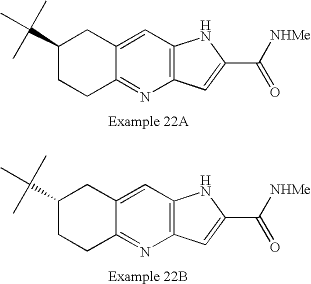

Following a procedure similar to that of Example 3, Examples 22A and 22B were prepared from compounds 21A and 21B respectively. Example 22A, LCMS: MH⁺=286 and Example 22B, LCMS: MH⁺=286.

### Example 23

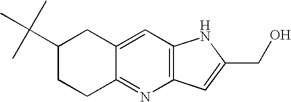

### Preparative Example 23

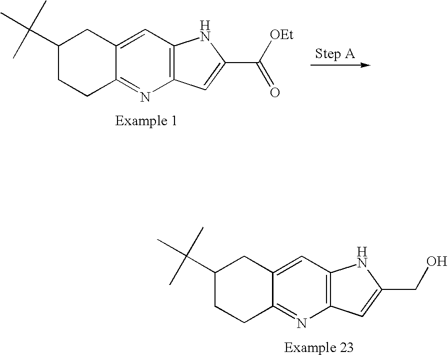

### Step A:

To a mixture of Example 1 (0.1 g, 0.33 mmol, 1 equiv) in THF (4 mL) was added 1M solution of lithium aluminum hydride in diethyl ether (0.4 mL, 0.4 mmol, 1.1 equiv) and the reaction mixture was heated at 60° C. for 0.5 hours. Cooled to room temperature and water (5 mL) was added carefully. The mixture was poured into EtOAc (200 mL) and washed with saturated aq. NaHCO₃ (100 mL). The organic layer was dried over Na₂SO₄, filtered and concentrated. The residue was purified by flash chromatography eluting with 10% MeOH/EtOAc to give the title compound Example 23 (0.030 g). LCMS: MH⁺=259.

### Example 24

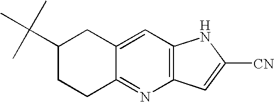

### Preparative Example 24

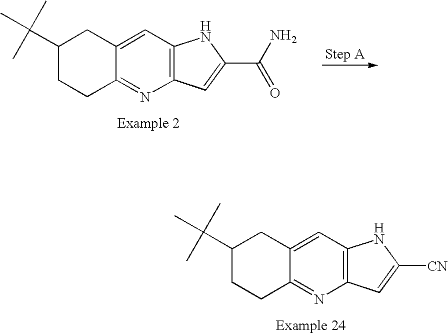

### Step A:

To a mixture of Example 2 (0.05 g, 0.19 mmol, 1 equiv) in pyridine (1 mL) at 0° C. was added POCl₃ (0.019 mL, 0.2 mmol, 1.1 equiv). Warmed to room temperature and stirred for 0.5 hours. Additional amount of POCl₃ (0.1 mL) was added to the reaction mixture and stirred at room temperature for 1 hour. Quenched with water (2 mL) and poured into CH₂Cl₂ (200 mL) and washed with saturated aq. NaHCO₃ (100 mL). The organic layer was dried over Na₂SO₄, filtered and concentrated. The residue was purified by flash chromatography eluting with 1/1 EtOAc/hexane to give the title compound Example 24 (0.007 g). LCMS: MH⁺=254.

### Example 25

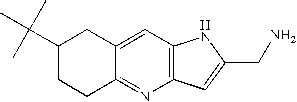

### Preparative Example 25

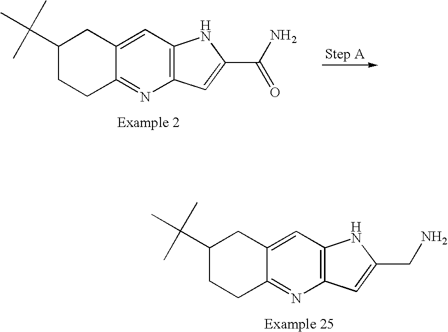

### Step A:

To AlCl₃ (0.045 g, 0.33 mmol, 1.5 equiv) was added 1M solution of lithium aluminum hydride in diethyl ether (0.99 mL, 0.99 mmol, 4.5 equiv) at 0° C. and stirred for 5 minutes. Example 2 (0.06 g, 0.22 mmol, 1 equiv) was added to the reaction mixture followed by THF (3 mL). Reaction mixture was warmed to room temperature and stirred for 18 hours. Saturated aq. Na,K tartarate (5 mL) was carefully added to the reaction mixture and stirred for 10 minutes. Poured into EtOAc (150 mL) and washed with saturated aq. NaHCO₃ (100 mL) followed by brine (100 mL). The organic layer was dried over Na₂SO₄, filtered and concentrated. The residue was purified by flash chromatography eluting with 7% MeOH(NH₃)/EtOAC to give the title compound Example 25 (0.06 g). LCMS: MH⁺=258.

### Example 26

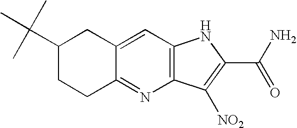

### Preparative Example 26

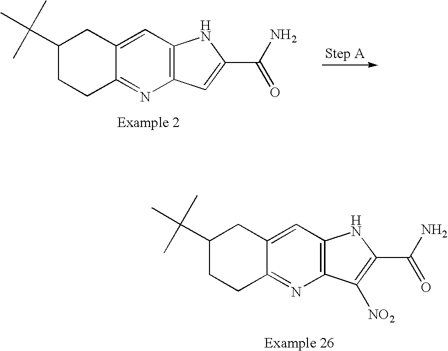

### Step A:

A mixture of Example 2 (0.1 g, 0.37 mmol) and fuming nitric acid (3 mL) was stirred at room temperature for 1 hour. Poured slowly into ice. Neutralize carefully with saturated aq. NaHCO₃ to pH 6-7 and poured into CH₂Cl₂ (200 mL). The organic layer was separated, dried over Na₂SO₄, filtered and concentrated. The residue was purified by flash chromatography eluting with EtOAC to give the title compound Example 26 (0.09 g). LCMS: MH⁺=317.

### Example 27

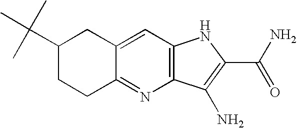

### Preparative Example 27

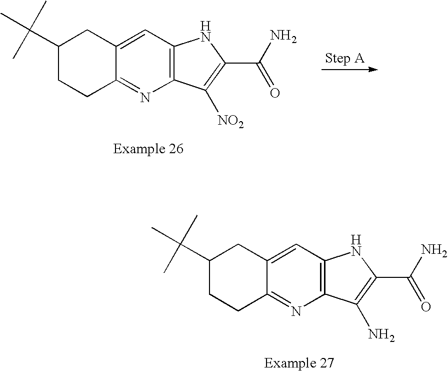

### Step A:

A mixture of Example 26 (0.09 g, 0.28 mmol), 20% Pd(OH)₂—C (0.06 g) and MeOH (10 mL) was stirred in a hydrogen ballon atmosphere at room temperature for 1 hour. Filtered the catalyst over celite, washed with MeOH and concentrated. The residue was purified by flash chromatography eluting with 5% MeOH/EtOAC to give the title compound Example 27 (0.06 g). LCMS: MH⁺=287.

### Example 28

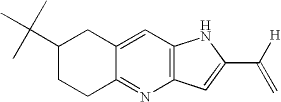

### Preparative Example 28

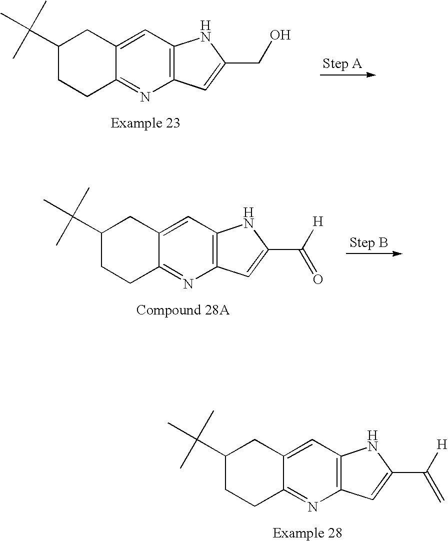

### Step A:

A mixture of Example 23 (0.13 g, 0.5 mmol, 1 equiv), MnO₂ (0.53 g, 6 mmol, 12 equiv) and CHCl₃ (5 mL) was stirred at room temperature for 1.5 hour. The mixture was purified by flash chromatography eluting with 60% EtOAc/hexane to give the compound 28A (0.07 g).

### Step B:

To a mixture of methyltriphenylphosphonium bromide (0.29 g, 0.82 mmol, 3 equiv) in toluene (7 mL) was added 0.5M solution of KHMDS in toluene (1.35 mL, 0.675 mmol, 2.5 equiv) at 0° C. Reaction mixture was stirred at 0° C. for 0.5 hour. A mixture of the compound 28A (0.07 g, 0.27 mmol, 1 equiv) in toluene (4 mL) was added to the reaction mixture 0° C. and stirred for 10 minutes. Warmed to room temperature and stirred for 30 minutes.

Quenched with saturated aq. NaHCO₃ and poured into EtOAc (200 mL). The organic layer was separated, dried over Na₂SO₄, filtered and concentrated. The residue was purified by flash chromatography eluting with 40% EtOAc/hexane to give the Example 28 (0.04 g). LCMS: MH⁺=255.

### Example 29

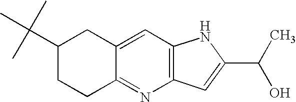

### Preparative Example 29

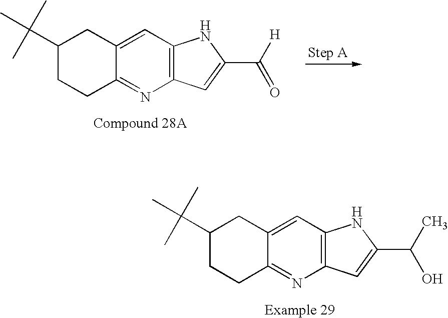

### Step A:

To a mixture of Compound 28A (0.1 g, 0.39 mmol, 1 equiv) in THF (4 mL) was added 1M solution of MeMgBr in THF (0.82 mL, 0.82 mmol, 2.1 equiv) at −78° C. and stirred for 20 minutes. Warmed to 0° C. and stirred for 1 hour. The reaction mixture was cooled to −78° C. and 1M solution of MeMgBr in THF (0.6 mL) was added and stirred for 10 minutes. Warmed to 0° C. and stirred for 0.5 hour. The reaction mixture was quenched with saturated aq. NH₄Cl and poured into EtOAc (200 mL). The organic layer was separated, washed with brine (100 mL), dried over Na₂SO₄, filtered and concentrated. The residue was purified by flash chromatography eluting with 5% MeOH/EtOAc to give the Example 29 (0.09 g). LCMS: MH⁺=273.

### Example 30

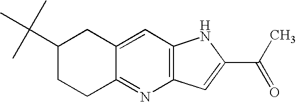

### Preparative Example 30

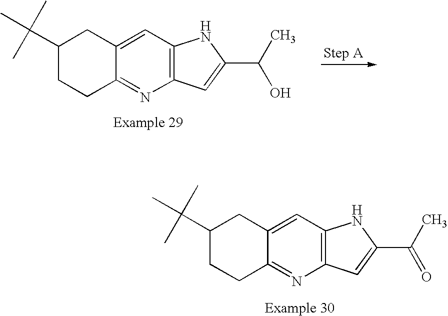

### Step A:

A mixture of Example 29 (0.09 g, 0.33 mmol, 1 equiv), MnO₂ (0.35 g, 4 mmol, 12 equiv) and CHCl₃ (5 mL) was stirred at room temperature for 1 hour. The mixture was purified by flash chromatography eluting with 60% EtOAc/hexane to give the Example 30 (0.08 g). LCMS: MH⁺=271.

### Example 31

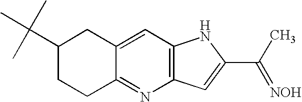

### Preparative Example 31

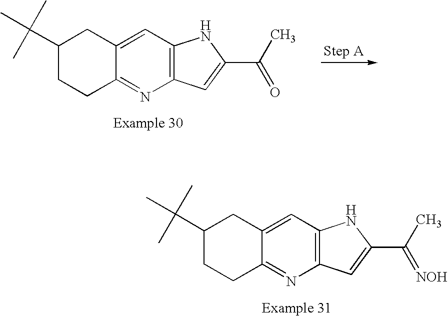

### Step A:

A mixture of Example 30 (0.09 g, 0.33 mmol, 1 equiv), hydroxylamine hydrochloride (0.092 g, 1.33 mmol, 4 equiv) and pyridine (4 mL) was stirred at room temperature for 18 hours. The mixture was concentrated and purified by flash chromatography eluting with 1/1 EtOAc/hexane to give the Example 31 (0.07 g). LCMS: MH⁺=286.

### Example 32

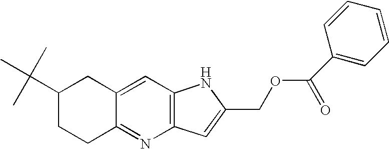

### Preparative Example 31

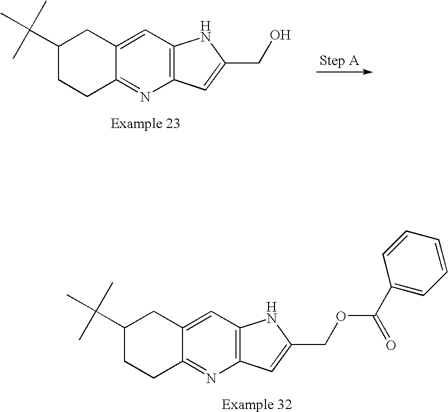

### Step A:

To a solution of Example 23 (0.1 g, 0.39 mmol, 1 equiv) in dry THF (5 mL) was added triethylamine (0.1 mL, 0.74 mmol, 1.9 equiv). The reaction mixture was cooled to 0° C. and benzoyl chloride (0.067 mL, 0.58 mmol, 1.5 equiv) was added. Reaction mixture was warmed to room temperature and stirred for 18 hours. Poured into CH₂Cl₂ (150 mL) and washed with saturated aq. NaHCO₃ (100 mL). The organic layer was dried over Na₂SO₄, filtered and concentrated. The residue was purified by flash chromatography eluting with 1/4 EtOAc/hexane followed by 1/1 EtOAc/hexane to give the product Example 32 (0.07 g). LCMS: MH⁺=363.

### Example 33

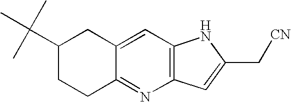

### Preparative Example 33

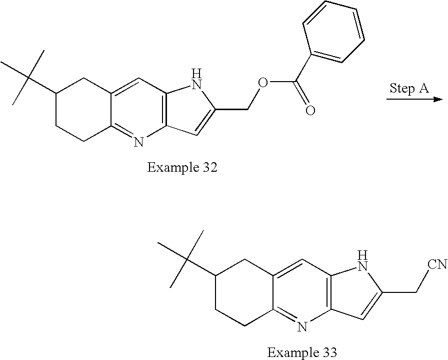

### Step A:

A mixture of Example 32 (0.07 g, 0.193 mmol, 1 equiv), potassium cyanide (0.038 g, 0.58 mmol, 3 equiv) and DMSO (3 mL) was heated at 64° C. for 4 hours. Cooled to room temperature and poured into CH₂Cl₂ (200 mL) and washed with saturated aq. NaHCO₃ (100 mL). The organic layer was dried over Na₂SO₄, filtered and concentrated. The residue was purified by flash chromatography eluting with 1/9 EtOAc/hexane to give the product Example 33 (0.03 g). LCMS: MH⁺=268.

### Example 34

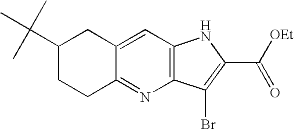

### Preparative Example 34

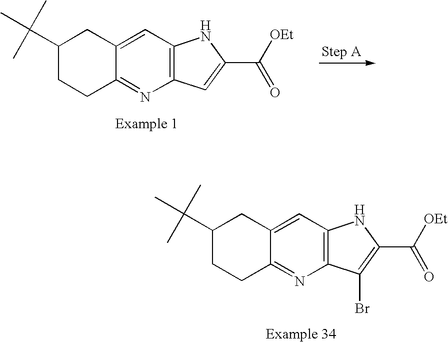

### Step A:

To a mixture of Example 1 (0.1 g, 0.332 mmol, 1 equiv) in dry DMF (4 mL) at 0° C. was added NBS (0.07 g, 0.39 mmol, 1.18 equiv) and the reaction mixture was stirred at 0° C. for 1.5 hours. Poured into EtOAc (200 mL) and washed with saturated aq. NaHCO₃ (100 mL). The organic layer was dried over Na₂SO₄, filtered and concentrated. The residue was purified by flash chromatography eluting with 1/2 EtOAc/hexane to give the product Example 34 (0.06 g). LCMS: M+2H⁺=381.

### Example 35

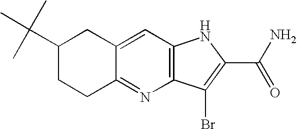

Example 35 was prepared from the Example 34 using the same procedure as described for the preparation of the Example 2 from the Example 1. LCMS: M+2H⁺=352.

### Examples 36 and 37

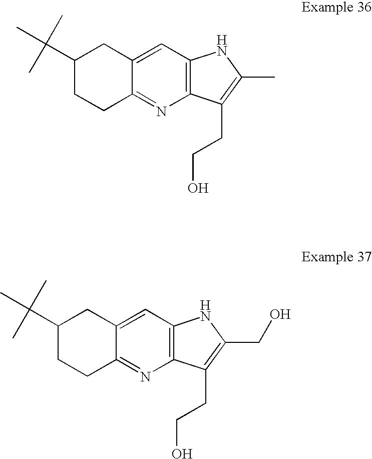

### Preparative Examples 36 and 37

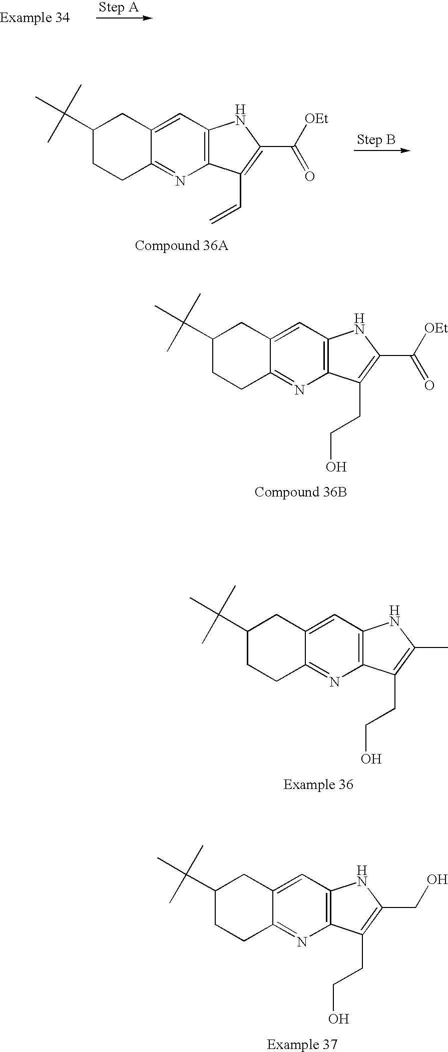

### Step A:

To a mixture of Example 34 (0.64 g, 1.69 mmol, 1 equiv) in dry DMF (10 mL) was added tributyl(vinyl)tin (1.48 mL, 5.06 mmol, 3 equiv) followed by tetrakis(triphenylphosphine)palladium(0) (0.47 g, 0.4 mmol, 0.24 equiv) and the reaction mixture was heated at 100° C. for 18 hours. Cooled to room temperature and a saturated solution of KF in MeOH (5 mL) was added and stirred for 1.5 hours. Poured into CH₂Cl₂ (200 mL) and washed with water (100 mL). The organic layer was dried over Na₂SO₄, filtered and concentrated. The residue was purified by flash chromatography eluting with 30% Et₂O/hexane to give the product Compound 36A (0.6 g).

### Step B:

To a mixture of Compound 36A (0.12 g, 0.37 mmol, 1 equiv) in dry THF (5 mL) was added borane-methyl sulfide complex (0.2 mL, 2.6 mmol, 7.1 equiv) and the reaction mixture was stirred at room temperature for 18 hours. To the reaction mixture was added aq. 1N NaOH (2 mL) followed by 30% hydrogen peroxide (2 mL) and stirred at room temperature for 1 hour. Poured into CH₂Cl₂ (200 mL) and washed with water (100 mL). The organic layer was dried over Na₂SO₄, filtered and concentrated. The residue was purified by flash chromatography eluting with EtOAc followed by 5% MeOH/EtOAc to give the products; Compound 36B (0.03 g), Example 37 (0.02 g) and Example 38 (0.15 g). Example 36, LCMS: MH⁺=287 and Example 37, LCMS: MH⁺=303.

### Example 38

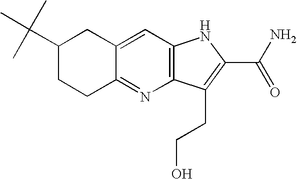

The Example 38 was prepared from the Compound 36B using the same procedure as described for the preparation of the Example 2 from the Example 1. LCMS: MH⁺=331.

### Examples 39

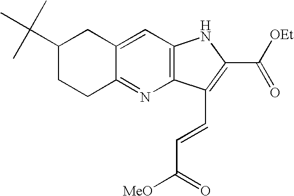

### Preparative Example 39

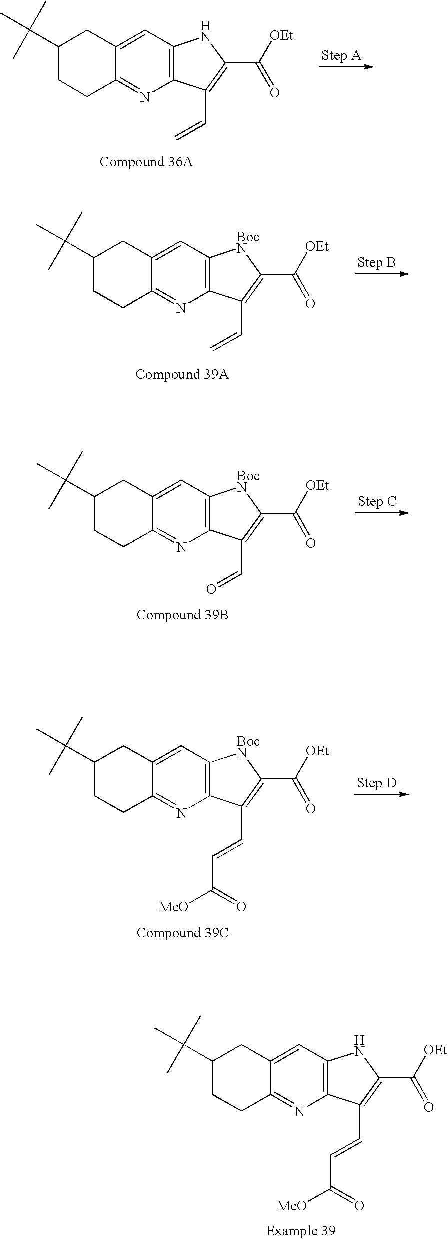

### Step A:

A mixture of Compound 36A (0.22 g, 0.67 mmol, 1 equiv), CH₂Cl₂ (10 mL), t-Boc₂O (0.441 g, 2 mmol, 3 equiv), DMAP (2 mg) and triethylamine (0.2 mL, 2 mmol, 3 equiv) and stirred at room temperature for 18 hours. Poured into CH₂Cl₂ (200 mL) and washed with saturated aq. NaHCO₃ (100 mL). The organic layer was dried over Na₂SO₄, filtered and concentrated. The residue was purified by flash chromatography eluting with 40% Et₂O/hexane to give the product Compound 39A (0.23 g).

### Step B:

A mixture of Compound 39A (0.12 g, 0.28 mmol, 1 equiv), 9/1 MeOH/CH₂Cl₂ (10 mL) was cooled to −78° C. and a stream of ozone was passed for 5 minutes. Dimethyl sulfide (1 mL) was added to the reaction mixture. The reaction mixture warmed to room temperature and stirred for 4 hours. Poured into CH₂Cl₂ (200 mL) and washed with saturated aq. NaHCO₃ (100 mL). The organic layer was dried over Na₂SO₄, filtered and concentrated to give the crude product Compound 39B which was used in the next reaction without further purification.

### Step C:

To a mixture of methyl diethylphosphonacetate (0.154 mL, 0.84 mmol, 3 equiv) in dry THF (5 mL) at 0° C. was added a mixture of 60% sodium hydride in mineral oil (0.034 g, 0.84 mmom, 3 equiv) and stirred at 0° C. for 25 minutes. The reaction mixture was added via syringe to the Compound 39B (0.12 g, 0.28 mmol, 1 equiv) and stirred at room temperature for 1.5 hours. Quenched with water (2 mL). Poured into CH₂Cl₂ (200 mL) and washed with saturated aq. NaHCO₃ (100 mL). The organic layer was dried over Na₂SO₄, filtered and concentrated. The residue was purified by flash chromatography eluting with 40% Et₂O/hexane to give the product Compound 39C (0.12 g).

### Step D:

A mixture of the Compound 39C (0.12 g, 0.25 mmol, 1 equiv), CH₂Cl₂ (10 mL) and trifluoroacetic acid (0.57 mL, 7.4 mmol, 30 equiv) was stirred at room temperature for 72 hours. Diluted with CH₂Cl₂ (200 mL) and washed with saturated aq. NaHCO₃ (100 mL). The organic layer was dried over Na₂SO₄, filtered and concentrated to give the product Example 39 (0.09 g). LCMS: MH⁺=385.

### Example 40

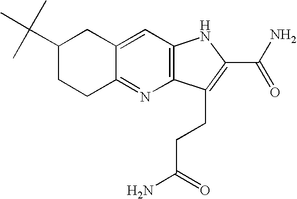

### Preparative Example 40

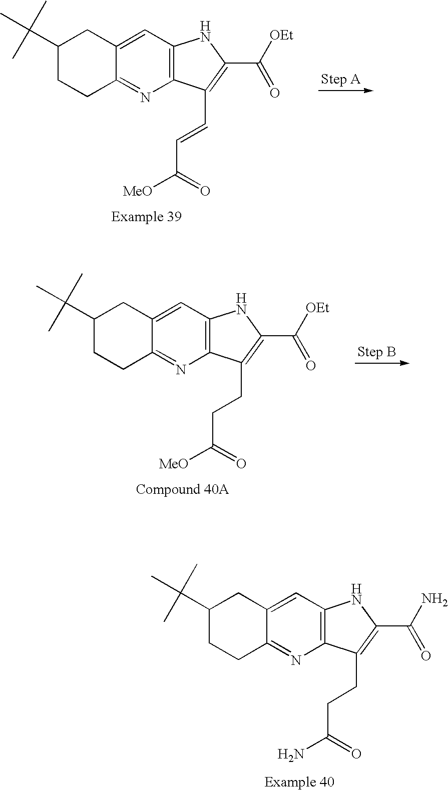

### Step A:

A mixture of Example 39 (0.09 g, 0.28 mmol), 20% Pd(OH)₂—C (0.04 g) and MeOH (10 mL) was stirred in a hydrogen ballon atmosphere at room temperature for 1 hour. Filtered the catalyst over celite, washed with MeOH and concentrated to give the crude product Compound 40A (0.06 g) which was used in next reaction without further purification.

### Step B:

The Compound 40A was converted to the Example 40 using the same procedure as described for the preparation of the Example 2 from the Example 1. LCMS: MH⁺=343.

### Example 41

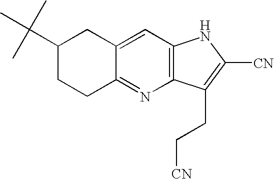

### Preparative Example 41

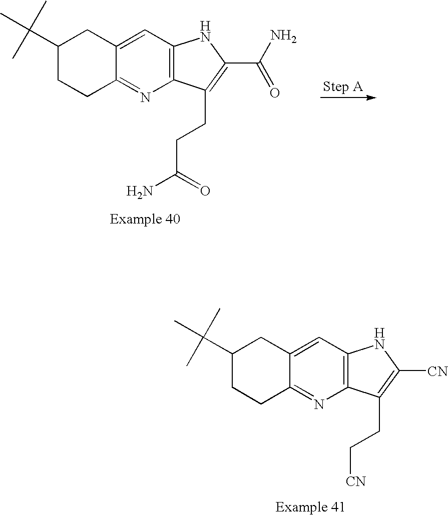

### Step A:

To a mixture of Example 40 (0.06 g, 0.18 mmol) in dry THF (5 mL) was added (methoxycarbonylsulfamoyl)triethylammonium hydroxide, inner salt (0.251 g, 1.05 mmol, 6 equiv) in four portions over two hours time period. The reaction mixture was stirred at room temperature for 18 hours. The reaction mixture was purified by flash chromatography eluting with 1/1 EtOAc/hexane followed by 40% EtOAc/hexane to give the products; Example 41 (0.01 g). LCMS: MH⁺=307.

### Example 42

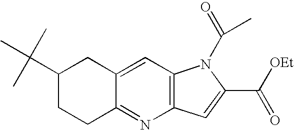

### Preparative Example 42

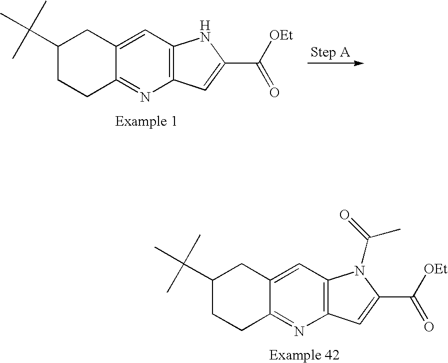

### Step A:

A mixture of Example 1 (0.32 g, 1.07 mmol, 1 equiv), acetic anhydride (0.25 mL, 2.66 mmol, 2.5 equiv), 4-(dimethylamino)pyridine (0.014 g, 0.12 mmom, 0.11 equiv) and CH₂Cl₂ (10 mL) was stirred at room temperature for 96 hours. Concentrated and purified by flash chromatography eluting with 1/1 EtOAc/hexane to give the title compound Example 42 (0.22 g). LCMS: MH⁺=343.

### Example 43

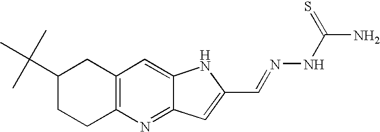

### Preparative Example 43

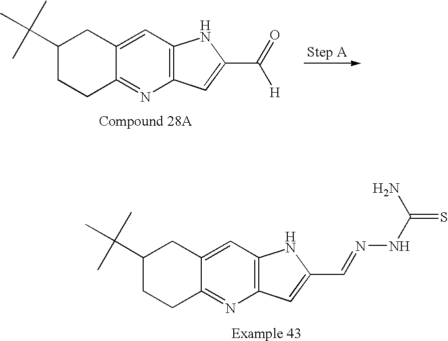

### Step A:

A mixture of Compound 28A (30 mg, 0.12 mmol) and thiosemicarbazide (107 mg, 1.2 mmol) were stirred in water/ethanol (3 ml/7 ml) with 1 drop conc. Hydrochloric acid at r.t. overnight. Ethyl acetate and water were added. The mixture was quenched with potassium carbonate. Layers were separated and the organic layer was washed with water, dried (MgSO₄) and filtered. Removal of solvents in vacuum gave yellow solid. The solid was washed with ether to give Example 43 as yellow solid (8 mg, 20%). LCMS: MH⁺=330.

### Example 44

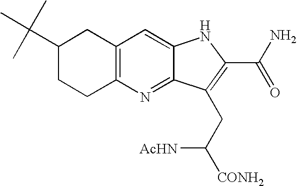

### Preparative Example 44

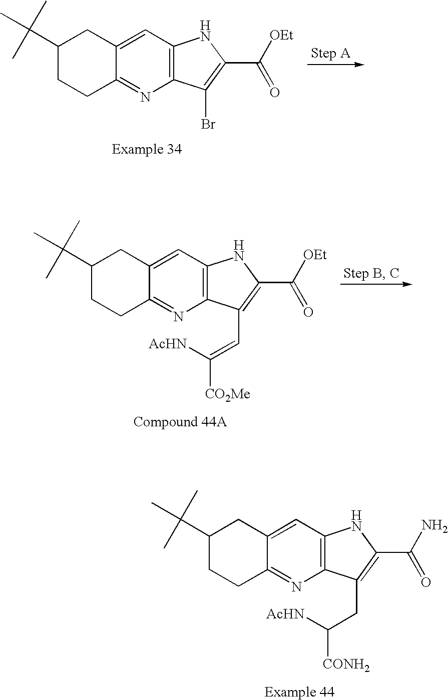

### Step A:

A mixture of Example 34 (0.1 g, 0.26 mmol), sodium acetate (0.085 g, 1.04 mmol, 4 equiv), methyl 2-acetamidoacrylate (0.076 g, 0.53 mmol, 2 equiv), dichlorobis(triphenylphosphine)palladium (II) (0.00183 g, 0.026 mmol, 0.1 equiv) and 2/1 Et₃N/DMF (3 mL) was heated at 130° C. for 4 hours. Cooled to room temperature and filtered through celite, washed with EtOAc (100 mL). The filtrate was washed with saturated aq. NaHCO₃ (100 mL). The organic layer was dried over Na₂SO₄, filtered and concentrated. The residue was purified by flash chromatography eluting with 30% EtOAc/hexane followed by 60% EtOAc/hexane to give the product Compound 44A (0.02 g).

### Step B:

The Compound 44A was converted to the Example 44 using the same procedure as described for the preparation of the Example 40 from the Example 39 LCMS: MH⁺=400.

### Example 45

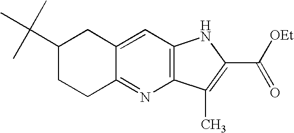

### Preparative Example 45

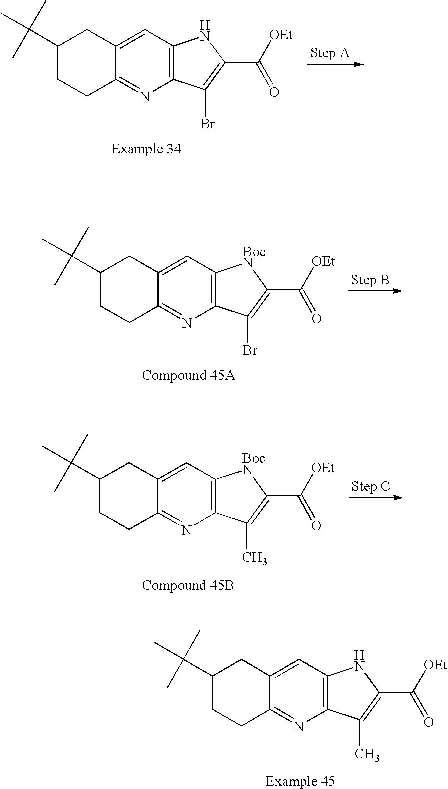

### Step A:

The Example 34 was converted to the Compound 45A using the same procedure as described for the preparation of the Compound 39A from the Compound 36A.

### Step B:

A mixture of Compound 45A (0.1 g, 0.21 mmol, 1 equiv), K₂CO₃ (0.086 g, 0.63 mmol, 3 equiv), methyl boronic acid (0.038 g, 0.63 mmol, 3 equiv), Pd(PPh3)4 (0.049 g, 0.042 mmol, 0.2 equiv) and toluene (5 mL) was heated at 80° C. for 18 hours. Cooled to room temperature and diluted with CH₂Cl₂ (200 mL) and washed with water (100 mL). The organic layer was dried over Na₂SO₄, filtered and concentrated. The residue was purified by flash chromatography eluting with 30% Et₂O/hexane to give the product Compound 45B (0.08 g).

### Step C:

The Compound 45B was converted to the Example 45 using the same procedure as described for the preparation of the Example 39 from the Compound 39C. LCMS: MH⁺=315.

### Example 46

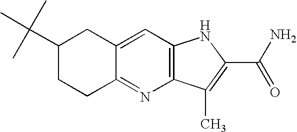

The Example 46 was prepared from the Example 45 using the same procedure as described for the preparation of the Example 2 from the Example 1. LCMS: MH⁺=286.

### Example 47

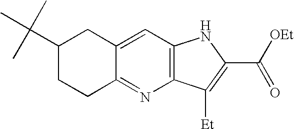

The Example 47 was prepared from the Compound 36A using the same procedure as described for the preparation of the Compound 40 from the Example 39 LCMS: MH⁺=329.

### Example 48

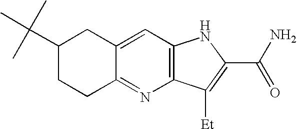

The Example 48 was prepared from the Example 47 using the same procedure as described for the preparation of the Example 2 from the Example 1. LCMS: MH⁺=300.

### Example 49

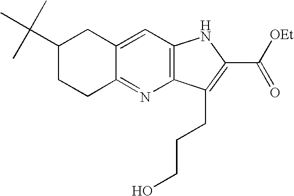

### Preparative Example 49

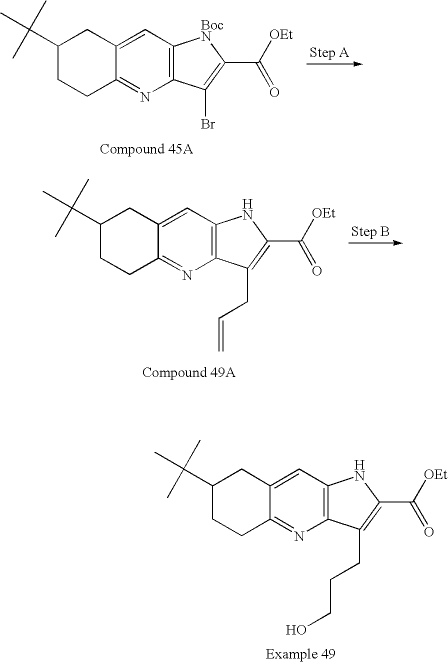

### Step A:

The Compound 45A was converted to the Compound 49A using the same procedure as described for the preparation of the Compound 36A from the Example 34, but using tributyl(allyl)tin in place of tributyl(vinyl)tin.

### Step B:

The Compound 49A was converted to the Example 49 using the same procedure as described for the preparation of the Compound 36B from the Compound 36A. LCMS: MH⁺=359.

### Example 50

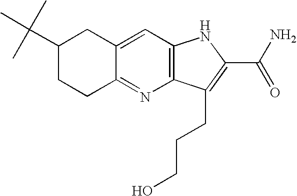

The Example 50 was prepared from the Example 49 using the same procedure as described for the preparation of the Example 2 from the Example 1. LCMS: MH⁺=330.

### Example 51

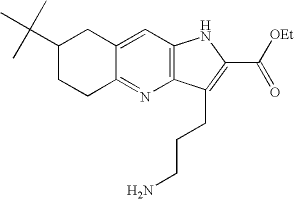

### Preparative Example 51

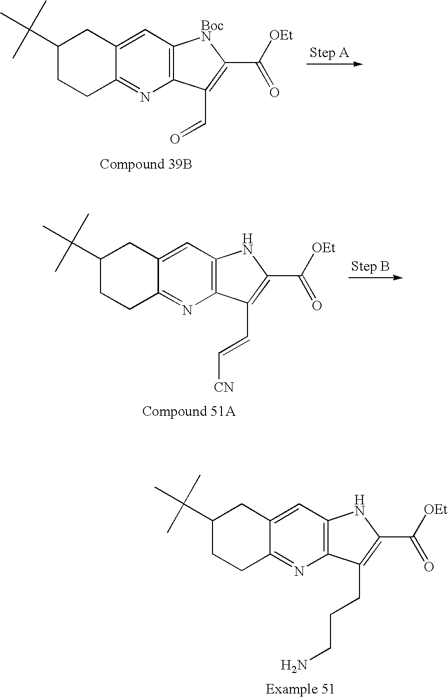

### Step A:

The Compound 39B was converted to the Compound 51A using the same procedure as described for the preparation of the Compound 39C from the Compound 39B, but using diethyl (cyanomethyl)phosphonate in place of methyl diethylphosphonoacetate.

### Step B:

A mixture of the Compound 51A (0.1 g, 0.28 mmol), 10% Pd—C (0.1 g) and EtOH (10 mL) was stirred in a hydrogen parr shaker at 60 psi for 72 hours. Filtered the catalyst over celite, washed with MeOH and concentrated. Taken the residue in 3/1 MeOH/THF (4 mL), cooled to −5° C. and cobalt (II) chloride hydrate (0.037 g) was added followed by sodium borohydride (0.011 g). Stirred at −5° C. for 15 minutes and quenched with 2N HCl (3 mL). Poured into EtOAc (200 mL) and washed with saturated aq. NaHCO₃ (100 mL). The organic layer was dried over Na₂SO₄, filtered and concentrated. The residue was purified by flash chromatography eluting with 10% MeOH(NH₃)/CH₂Cl₂ to give the product Example 51.

### Example 52

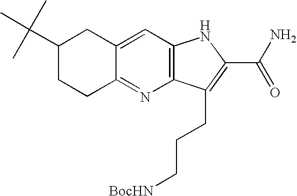

### Preparative Example 52

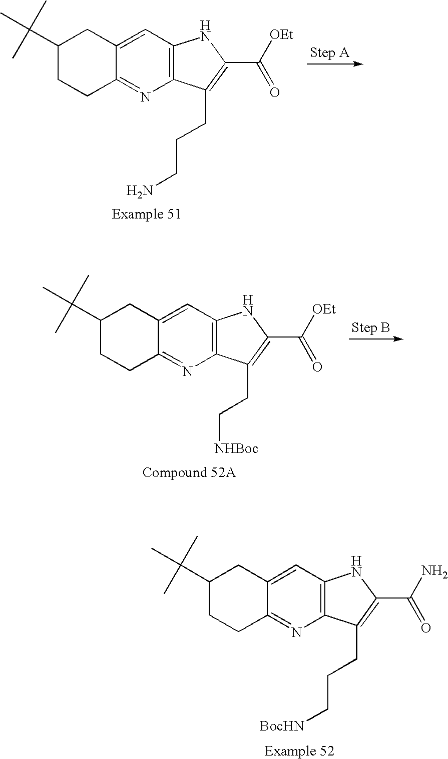

### Step A:

The Example 51 was converted to the Compound 52A using the similar procedure as described for the preparation of Compound 39A from the Compound 36A.

### Step B:

The Example 52 was prepared from the Compound 52A using the same procedure as described for the preparation of the Example 2 from the Example 1. LCMS: MH⁺=429.

### Example 53

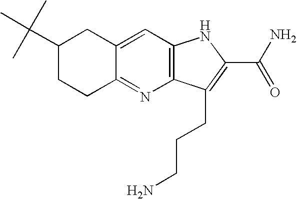

The Example 52 was converted to the Example 53 using the similar procedure as described for the preparation of the Example 39 from the Compound 39C. LCMS: MH⁺=329.

### Example 54

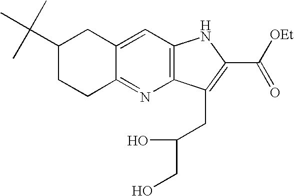

### Preparative Example 54

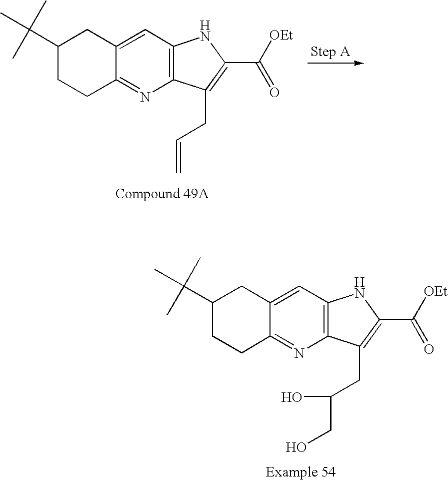

### Step A:

A mixture of the Compound 49A (0.1 g, 0.29 mmol, 1 equiv), potassium osmate dehydrate (0.016 g, 0.044 mmol, 0.15 equiv), 4-methylmorpholine N-oxide (0.051 g, 0.44 mmol, 1.5 equiv), acetone (6 mL) and water (2 mL) was stirred at room temperature for 18 hours. Poured into EtOAc (200 mL) and washed with water (100 mL). The organic layer was dried over Na₂SO₄, filtered and concentrated. The residue was purified by flash chromatography eluting with 5% MeOH/EtOAc to give the product Example 54 (0.05 g). LCMS: MH⁺=375.

### Example 55

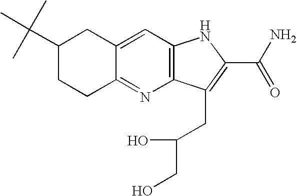

The Example 55 was prepared from the Example 54 using the same procedure as described for the preparation of the Example 2 from the Example 1. LCMS: MH⁺=346.

### Example 56

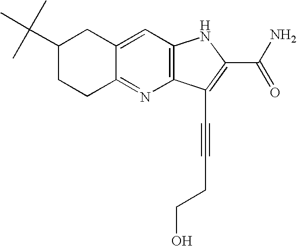

### Preparative Example 56

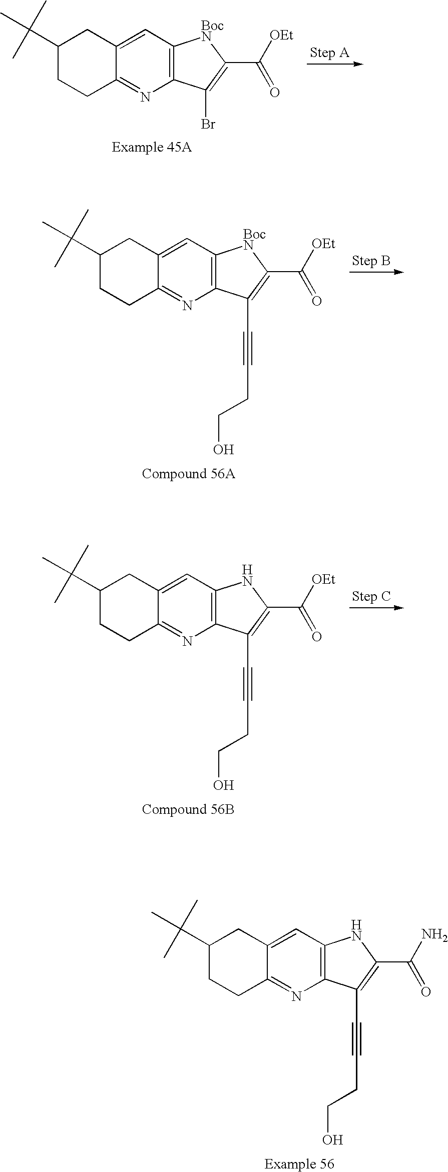

### Step A:

A mixture of Compound 45A (0.4 g, 0.84 mmol, 1 equiv), bytyn-1-ol (0.076 mL, 1 mmol, 1.2 equiv), copper (I) iodide (0.032 g, 0.168 mmol, 0.2 equiv), triethyl amine (0.132 mL, 0.092 mmol, 1.1 equiv), Pd(PPh₃)₄ (0.097 g, 0.084 mmol, 0.1 equiv) and DMF (8 mL) was heated at 80° C. for 4.5 hours. Cooled to room temperature, diluted with EtOAc (200 mL) and washed with water (2×100 mL) followed by brine (100 mL). The organic layer was dried over Na₂SO₄, filtered and concentrated. The residue was purified by flash chromatography eluting with 1/1 EtOAC/hexane followed by EtOAc to give the product Compound 56A (0.3 g).

### Step B:

The Compound 56A was converted to the Compound 56B using the same procedure as described for the preparation of the Example 39 from the Compound 39C. LCMS: MH⁺=315.

### Step C:

The Example 56 was prepared from the Compound 56B using the same procedure as described for the preparation of the Example 2 from the Example 1. LCMS: MH⁺=340.

### Example 57

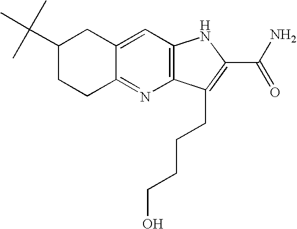

### Preparative Example 57

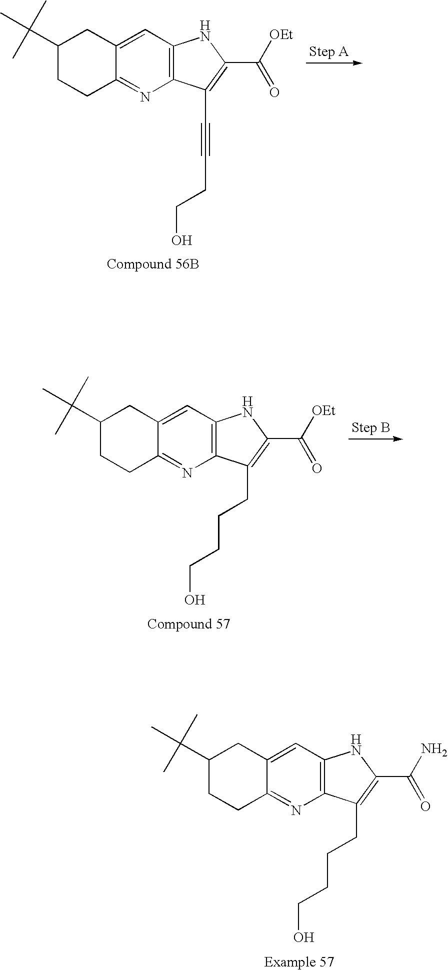

### Step A:

A mixture of the Compound 56B (0.130 g, 0.35 mmol), 10% Pd—C (0.050 g) and EtOH (10 mL) was stirred in a hydrogen parr shaker at 60 psi for 4 hours. Filtered the catalyst over celite, washed with MeOH and concentrated to give the crude product Compound 57 which was used in next reaction without further purification.

### Step B:

The Example 57 was prepared from the Compound 57 using the same procedure as described for the preparation of the Example 2 from the Example 1. LCMS: MH⁺=344.

### Example 58

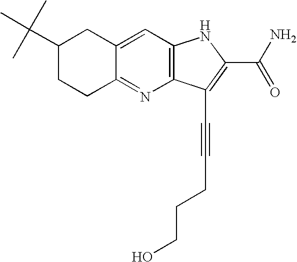

Compound 45A was converted to Example 58 using the same procedures as described for the preparation of the Example 56 from the Compound 45A, but using 4-pentyn-1-ol in place of 4-butyn-1-ol. LCMS: MH⁺=354.

### Example 59

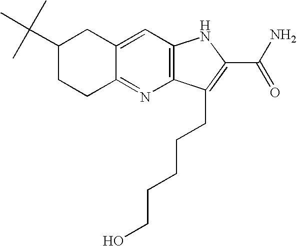

The Example 59 was prepared from the Example 58 using the same procedure as described for the preparation of the Example 57 from the Compound 56B. LCMS: MH⁺=358.

### Examples 60-61

The Example 47 and Compound 57 and were converted to Examples 60 and 61 respectively following a procedure similar to that of Example 5, but using ethylenediamine in place of 1,4-diaminobutane.

| Example | Structure | LCMS: MH⁺ |
|---|---|---|
| 60 | 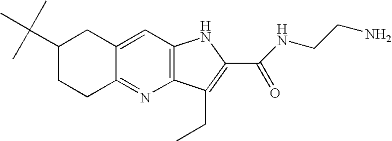 | 343 |
| 61 | 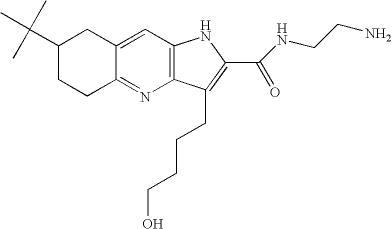 | 387 |

### Example 62

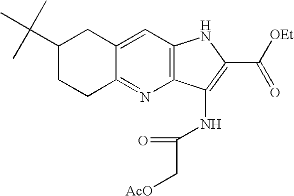

### Preparative Example 62

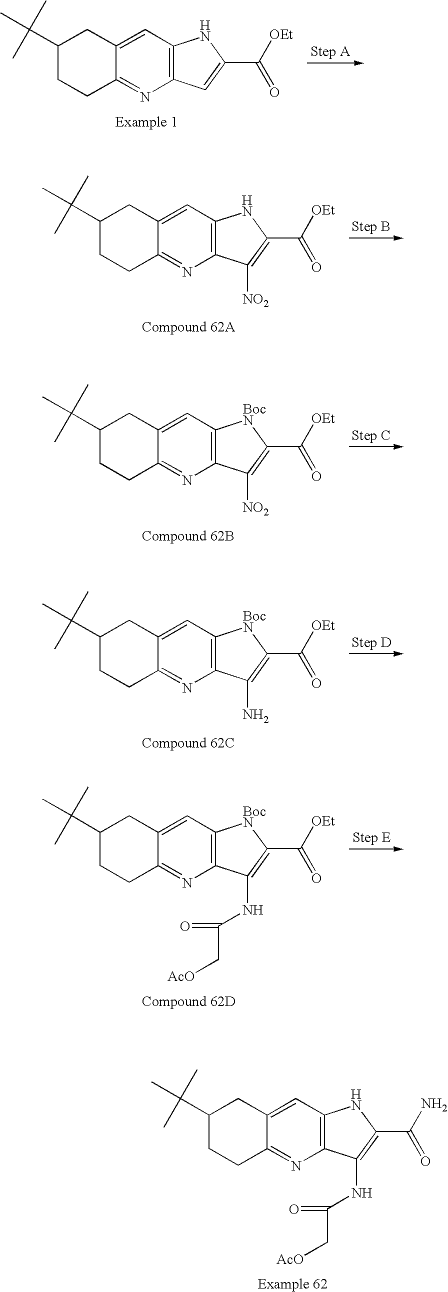

### Step A:

A mixture of the Example 1 (1.5 g, 4.99 mmol, 1 equiv), and fuming nitric acid (3 mL) was stirred at room temperature for 3 hours. Poured carefully into ice/saturated aq. NaHCO₃ mixture and extracted with CH₂Cl₂ (3×200 mL). The combined organic layers were dried over Na₂SO₄, filtered and concentrated to give the crude product Compound 62A which was used in next reaction without further purification.

### Step B:

The Compound 62A was converted to the Compound 62B using the same procedure as described for the preparation of the Compound 39A from the Compound 36A.

### Step C:

A mixture of Compound 62B (0.35 g, 0.78 mmol), 20% Pd(OH)₂—C (0.05 g) and MeOH (10 mL) was stirred in a hydrogen ballon atmosphere at room temperature for 1.5 hours. Filtered the catalyst over celite, washed with MeOH and concentrated. The residue was purified by flash chromatography eluting with 30% EtOAC/hexane to give the product Compound 62C (0.22 g).

### Step D:

To a mixture of Compound 62C (0.22 g, 0.53 mmol) in CH₂Cl₂ (7 mL) was added N,N-diisopropylethylamine (0.12 mL, 0.69 mmol, 1.3 equiv). The reaction mixture was cooled to 0° C. and acetoxyacetyl chloride (0.14 mL, 1.3 mmol, 1.3 equiv) was added. The reaction mixture was warmed to room temperature and stirred for 72 hours. Diluted with CH₂Cl₂ (200 mL) and washed with saturated aq. NaHCO₃ (100 mL). The organic layer was dried over Na₂SO₄, filtered and concentrated. The residue was purified by flash chromatography eluting with 40% EtOAC/hexane followed by 60% EtOAc to give the product Compound 62D (0.14 g).

### Step E:

The Compound 62D was converted to the Example 62 using the same procedure as described for the preparation of the Example 39 from the Compound 39C. LCMS: MH⁺=416.

### Example 63

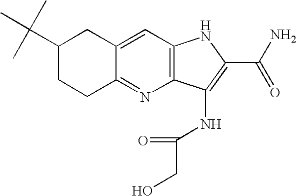

The Example 63 was prepared from the Example 62 using the same procedure as described for the preparation of the Example 2 from the Example 1. LCMS: MH⁺=345.

### Example 64

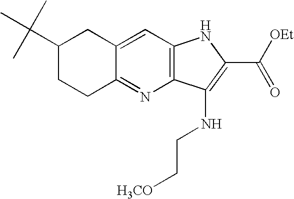

### Preparative Example 64

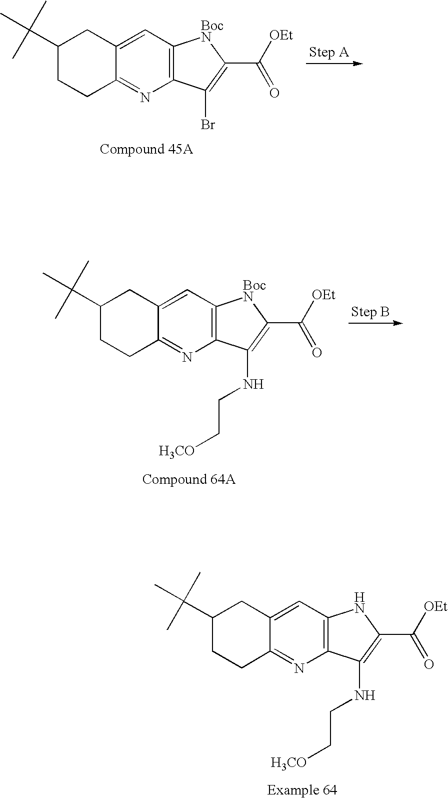

### Step A:

A mixture of the Compound 45A (0.24 g, 0.5 mmol, 1 equiv), cesium carbonate (0.229 g, 0.7 mmol, 1.4 equiv), BINAP (0.031 mg, 0.05 mmol, 0.1 equiv), Pd₂(dba)₃ (0.023 g, 0.025 mmol, 0.05 equiv), 2-methoxyethylamine (0.052 mL, 0.6 mmol, 1.2 equiv) and toluene (5 mL) was heated at 100° C. for 18 hours. Cooled to room temperature and purified by flash chromatography eluting with 35% Et₂O/hexane to give the product Compound 64A (0.04 g).

### Step B:

The Compound 64A was converted to the Example 64 using the same procedure as described for the preparation of the Example 39 from the Compound 39C. LCMS: MH⁺=374.

### Example 65

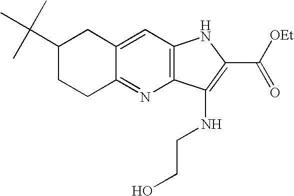

### Preparative Example 65

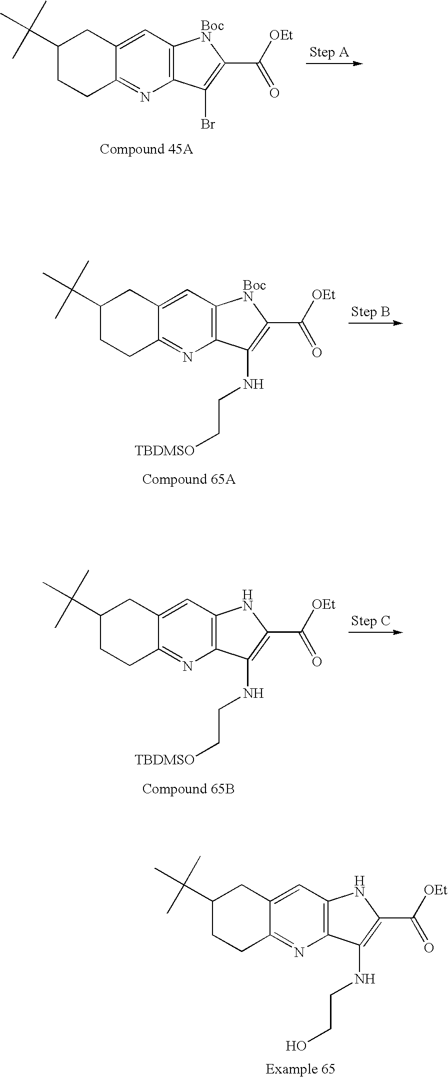

### Step A:

The Compound 45A was converted to the Compound 65A using the same procedure as described for the preparation of the Compound 64A from the Compound 45A, but using 2-trimethylsilanyloxy-ethylamine in place of 2-methoxyethylamine

### Step B:

The Compound 65A was converted to the Compound 65B using the same procedure as described for the preparation of the Example 39 from the Compound 39C.

### Step C:

A mixture of the Compound 65B (0.34 g, 0.72 mmol, 1 equiv), a 1M solution of tetrabutylammonium fluoride in THF (1.9 mL, 1.9 mmol, 2.6 equiv) and THF (10 mL) was stirred at room temperature for 18 hours. Diluted with EtOAc (200 mL) and washed with water (2×100 mL). The organic layer was dried over Na₂SO₄, filtered and concentrated. The residue was purified by flash chromatography eluting with 40% EtOAC/hexane to give the desired product Example 65 (0.09 g). LCMS: MH⁺=360.

### Example 66

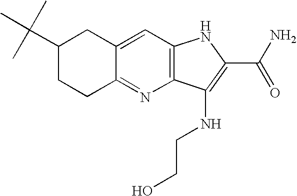

The Example 66 was prepared from the Example 65 using the same procedure as described for the preparation of the Example 2 from the Example 1. LCMS: MH⁺=331.

### Example 67

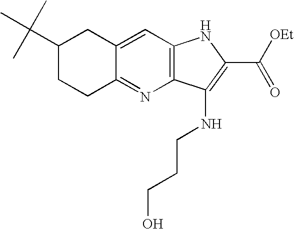

The Example 67 was prepared from the Compound 45A using the same procedure as described for the preparation of the Example 65 from the Compound 45A, but using 2-trimethylsilanyloxy-propylamine in place of 2-methoxyethylamine. LCMS: MH⁺=374.

### Example 68

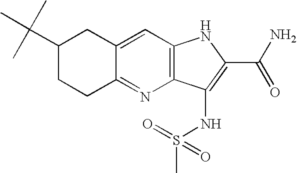

### Preparative Example 68

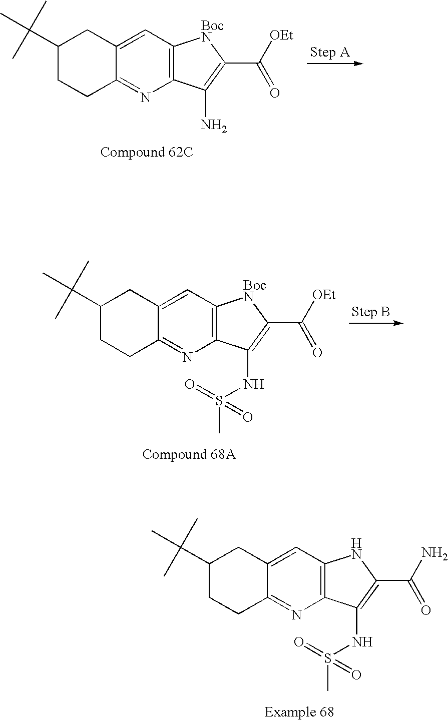

### Step A:

To a mixture of the Compound 62C (0.1 g, 0.24 mmol, 1 equiv) and pyridine (1 mL) was added methanesulfonyl chloride (0.075 mL, 0.96 mmol, 4 equiv) and the reaction mixture was stirred at room temperature for 18 hours. Poured into CH₂Cl₂ (200 mL) and washed with saturated aqueous NaHCO₃ (100 mL). The organic layer was dried over Na₂SO₄, filtered and concentrated. The residue was purified by flash chromatography eluting with 30% EtOAC/hexane to give the product Compound 68A (0.09 g).

### Step B:

The Compound 68A was converted to the Example 68 using the same procedure as described for the preparation of the Example 2 from the Example 1. LCMS: MH⁺=365.

### Example 69

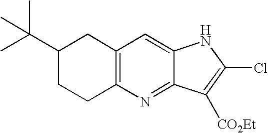

### Preparative Example 69

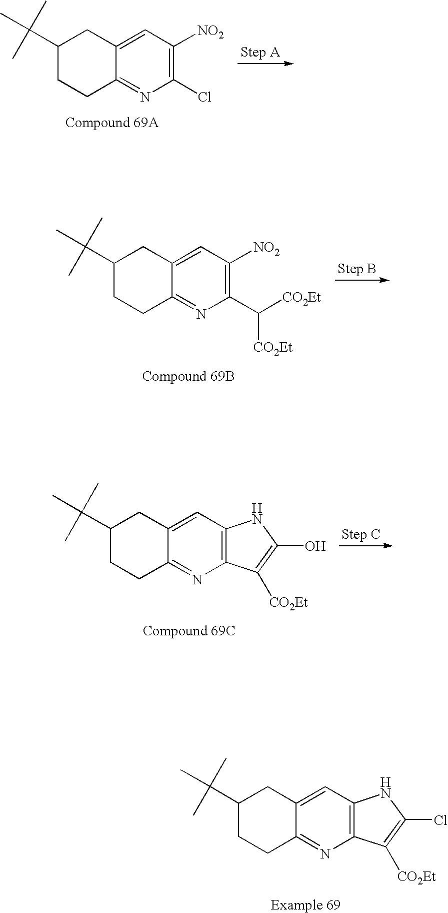

### Step A:

A suspension of NaH (776 mg, 19.4 mmol) in DMF (20 mL) at 0° C. under N₂, add diethyl malonate (2.95 ml, 19.4 mmol). Cooling bath was removed and the mixture was warmed to r.t. A solution of Compound 69A (1.74 g, 6.47 mmol) in DMF (ml) was added and the mixture was stirred at r.t. overnight. The mixture was quenched with saturated ammonium chloride solution and was diluted with water and ethyl acetate. Layers were separated and the aqueous layer was extracted with ethyl acetate (2×100 mL), dried (MgSO₄) and filtered. Removal of solvents in vacuum followed by column chromatography [hexanes-ethyl acetate, 9:1 (v/v)] gave Compound 69B as yellow oil (787 mg, 31%).

### Step B:

To a solution of Compound 69B (310 mg, 0.79 mmol) in acetic acid (5 mL) at r.t. zinc powder (513 mg, 7.9 mmol) was added in small portions. The suspension was heated at 80° C. for 2 hr. After being cooled to r.t., the solid was filtered through Celite. Solvents were then removed in vacuum. The residue was dissolved in ethyl acetate and was neutralized with saturated sodium bicarbonate solution. Layers were separated and the aqueous layer was extracted with ethyl acetate (2×100 mL), dried (MgSO₄) and filtered. Removal of solvents in vacuum gave yellow solid. The solid was washed with ether to give Compound 69C as yellow solid (212 mg, 85%).

### Step C:

Compound 69C (50 mg, 0.16 mmol) was dissolved in phosphorous oxychloride (0.5 ml) and the mixture was heated at 100° C. for 2 hr. After being cooled to r.t., ethyl acetate was added. The mixture was added to a mixture of ice/water carefully. Layers were separated and the aqueous layer was extracted with ethyl acetate (2×100 mL), dried (MgSO₄) and filtered. Removal of solvents in vacuum followed by column chromatography [hexanes-ethyl acetate, 1:2 (v/v)] gave Example 69 as yellow oil (46 mg, %). LCMS MH⁺=335.

### Example 70

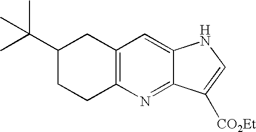

### Preparative Example 70

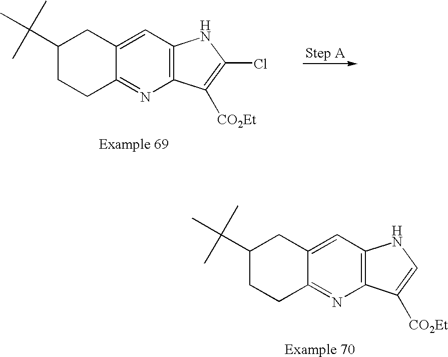

### Step A:

Example 69 (35 mg, 0.11 mmol) was dissolved in ethanol (10 mL) at r.t. and catalytic amount of Pd/C followed by triethylamine (204) were added. The mixture was stirred under hydrogen (balloon) overnight. The mixture was filtered through celite and solvents were removed in vacuum. Column chromatography [hexanes-ethyl acetate, 1:2 (v/v)] gave Example 70 as white solid (mg, %). LCMS: MH⁺=301.

### Example 71

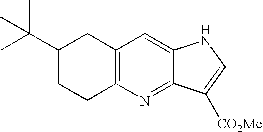

### Preparative Example 71

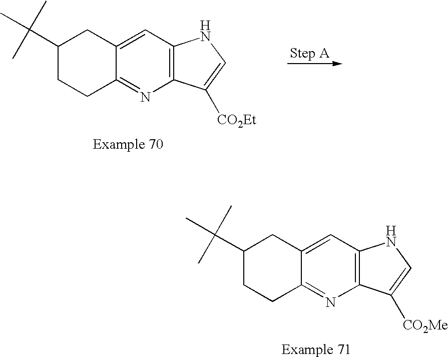

### Step A:

A solution of Example 70 (55 mg, 0.18 mmol) and a catalytic amount of sodium methoxide were stirred at reflux in methanol (10 ml) for 18 hours. The mixture was cooled at r.t. and solvents were removed in vacuum. The mixture was diluted with dichloromethane and water. Layers were separated and the aqueous layer was extracted with dichloromethane (2×100 mL), dried (MgSO₄) and filtered. Removal of solvents in vacuum gave white solid. The solid was washed with ether to give methyl ester Example 71 as white solid (47 mg, 90%). LCMS: MH⁺=287.

### Example 72

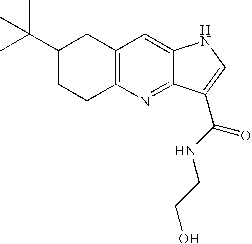

### Preparative Example 72

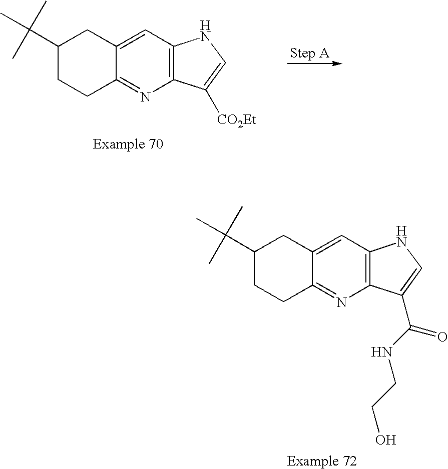

### Step A:

A solution of Example 70 (48 mg, 0.16 mmol), 2-hydroxylamine (1 ml) and a catalytic amount of sodium cyanide were heated in a sealed-tube at 120° C. overnight. After being cooled to r.t., the mixture was diluted with water and ethyl acetate. The organic layer was washed with water (2×100 mL), dried (MgSO₄) and filtered. Removal of solvents in vacuum gave white solid. The solid was washed with ether to give Example 72 as white solid (30 mg, 60%). LCMS: MH⁺=316.

### Example 73

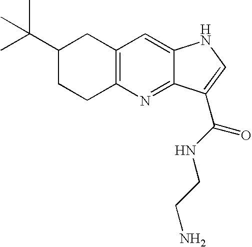

The Example 73 was prepared from Example 70 using the same procedure as described for the preparation of the Example 72 from Example 70. Example 73 was obtained as (7 mg, 55%). LCMS: MH⁺=315.

### Example 74

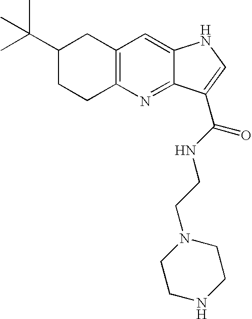

The Example 74 was prepared from Example 70 using the same procedure as described for the preparation of the Example 72 from Example 70. Example 74 was obtained as (30 mg, 45%). LCMS: MH⁺=384.

### Example 75

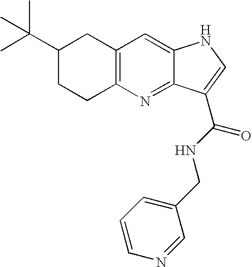

The Example 75 was prepared from Example 70 using the same procedure as described for the preparation of the Example 72 from Example 70. Example 75 was obtained as (31 mg, 52%). LCMS MH⁺=363.

### Example 76

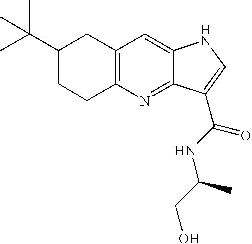

The Example 76 was prepared from Example 70 using the same procedure as described for the preparation of the Example 72 from Example 70. Example 76 was obtained as (27 mg, 48%). LCMS: MH⁺=330.

### Example 77

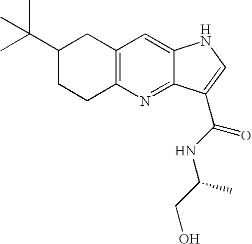

The Example 77 was prepared from Example 70 using the same procedure as described for the preparation of the Example 72 from Example 70. Example 82 was obtained as (24 mg, 43%). LCMS: MH⁺=330.

### Example 78

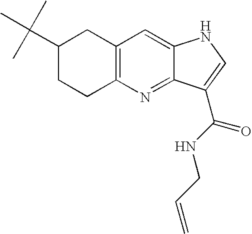

The Example 78 was prepared from Example 70 using the same procedure as described for the preparation of the Example 72 from Example 70. Example 78 was obtained as (48 mg, 53%). LCMS: MH⁺=312.

### Example 79

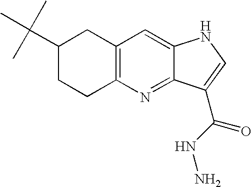

The Example 79 was prepared from Example 70 using the same procedure as described for the preparation of the Example 72 from Example 70. Example 78 was obtained as (28 mg, 53%). LCMS: MH⁺=287.

### Example 80

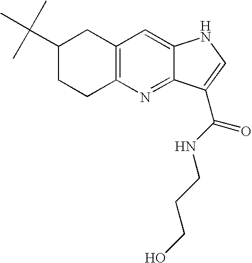

The Example 80 was prepared from Example 70 using the same procedure as described for the preparation of the Example 72 from Example 70. Example 80 was obtained as (18 mg, 30%). LCMS: MH⁺=330.

### Example 81

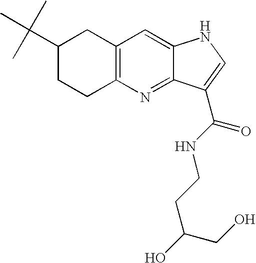

The Example 81 was prepared from Example 70 using the same procedure as described for the preparation of the Example 72 from Example 70. Example 81 was obtained as (23 mg, 40%). LCMS: MH⁺=346.

### Example 82

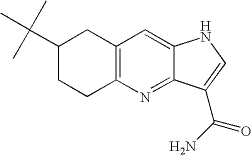

### Preparative Example 82

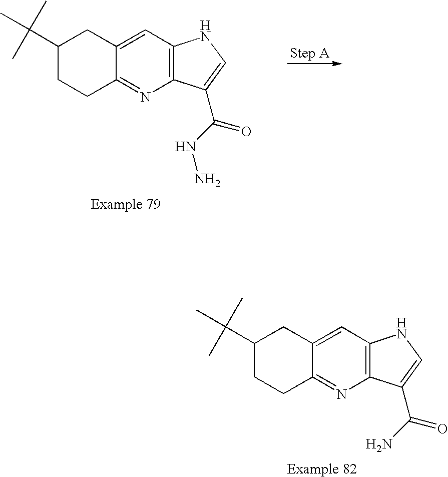

### Step A:

A mixture of Example 79 (15 mg, 0.052 mmol) and a catalytic amount of Raney Ni were heated at 100° C. in water for 1 hr. The mixture was cooled to r.t. and the solid was filtered through Celite. Ethyl acetate was added and layers were separated, dried (MgSO₄) and filtered. Removal of solvents in vacuum gave white solid. The solid was washed with ether to give Example 82 as white solid (11 mg, 74%). LCMS: MH⁺=272.

### Example 83

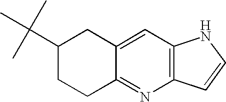

### Preparative Example 83

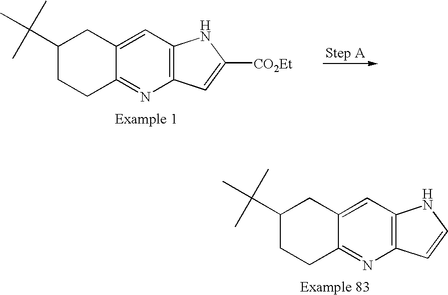

### Step A:

A mixture of Example 1 (100 mg, 0.052 mmol), NMP (3 mL), N-methyl piperazine (1 mL) was heated at 200° C. for 24 hours. The mixture was cooled to room temperature, poured into EtOAc (200 mL) and washed with water (100 mL). The organic layer was dried (Na₂SO₄), filtered and concentrated. The residue was purified by flash chromatography eluting with 10% MeOH/EtOAC to give the product Example 83 (0.01 g). LCMS: MH⁺=229.

### Example 84

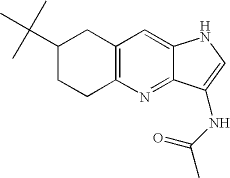

### Preparative Example 84

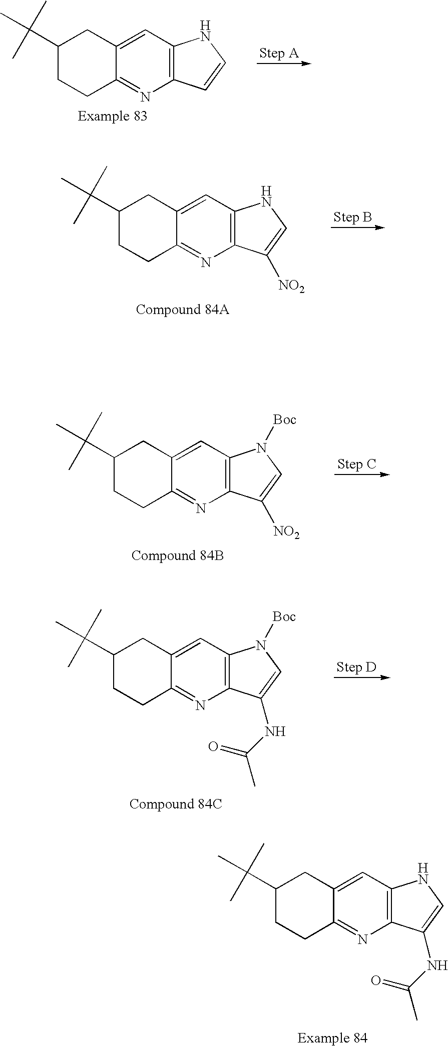

### Step A:

A mixture of Example 83 (610 mg, 2.67 mmol) in fuming nitric acid (10 ml) was stirred at r.t. for 30 min. The mixture was added slowly to a mixture of ethyl acetate/water/ice and was quenched carefully with potassium carbonate. Layers were separated and the aqueous layer was extracted with ethyl acetate (2×100 mL), dried (MgSO₄) and filtered. Removal of solvents in vacuum gave Compound 84A as brown solid. Compound 84A was used in the next step without further purification.

### Step B:

To a solution of Compound 84A (Step 1) in dichloromethane (20 ml), (Boc)₂O (1.2 g, 5.34 mmol) followed by triethylamine (1.1 ml, 8.01 mmol) were added. A catalytic amount of DMAP was added and the mixture was stirred at r.t. 3 hr. The mixture was quenched with saturated sodium bicarbonate solution. Layers were separated and the aqueous layer was extracted with dichloromethane (100×2), dried (MgSO₄) and filtered. Removal of solvents in vacuum followed by column chromatography (dichloromethane) gave Compound 84B as white solid (518 mg, 52%).

### Step C:

To a solution of Compound 84B (180 mg, 0.48 mmol) in methanol (ml), Pd(OH)₂/C (34 mg, 0.048 mmol), acetic anhydride (0.1 ml, 0.96 mmol) were added. The mixture was stirred under hydrogen (balloon) overnight. The mixture was filtered through Celite and solvents were removed in vacuum. Column chromatography (ethyl acetate) gave Compound 84C as yellow foam (122 mg, 66%).

### Step D:

To a solution of Compound 84C (50 mg, 0.13 mmol) in dichloromethane (5 ml), trifluoroacetic acid (0.1 ml) was added. The mixture was heated at reflux for 3 hr. After being cooled to r.t., solvents were removed in vacuum. Ethyl acetate was added and the mixture was quenched with saturated sodium bicarbonate solution. Layers were separated and the aqueous layer was extracted with dichloromethane (2×100 mL), dried (MgSO₄) and filtered. Removal of solvents in vacuum gave yellow solid. The solid was washed with ether to give Example 84 as yellow solid (19 mg, 50%). LCMS: MH⁺=286.

### Example 85

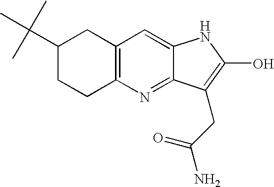

### Preparative Example 85

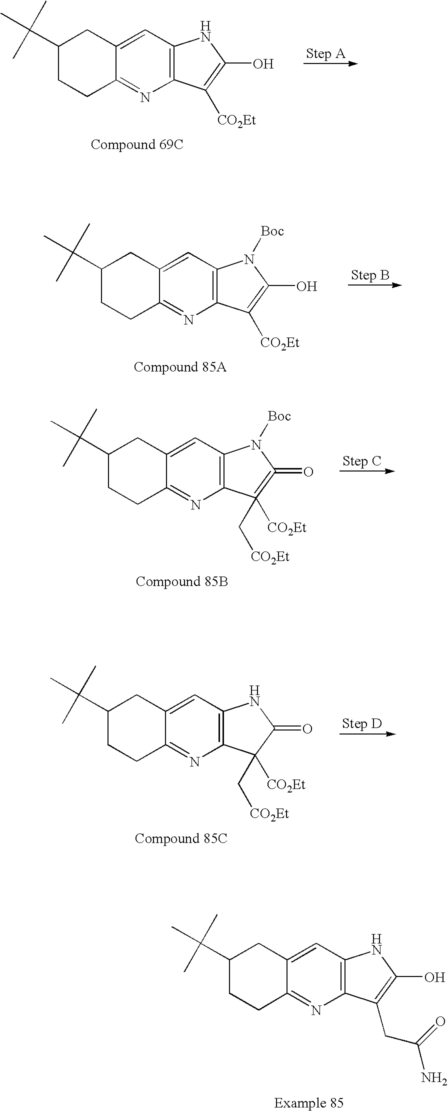

### Step A:

To a solution of Compound 69C (1.0 g, 3.16 mmol) in dichloromethane (20 ml), (Boc)₂O (2.1 g, 9.48 mmol) followed by triethylamine (1.33 ml, 9.48 mmol) were added. A catalytic amount of DMAP was added and the mixture was stirred at r.t. for 2 hr. The mixture was quenched with saturated sodium bicarbonate solution. Layers were separated and the aqueous layer was extracted with dichloromethane (2×150 mL), dried (MgSO₄) and filtered. Removal of solvents in vacuum followed by column chromatography (5% ethyl acetate in dichloromethane) gave Compound 85A as white solid (961 mg, 73%).

### Step B:

To a solution of Compound 85A (110 mg, 0.26 mmol) in DMF (5 ml) at r.t., potassium carbonate (183 mg, 1.32 mmol) followed by bromoethylacetate (0.06 ml, 0.53 mmol) were added. The mixture was stirred at r.t. overnight. The mixture was diluted with ethyl acetate and water. Layers were separated and the aqueous layer was extracted with dichloromethane (2×50 mL), dried

(MgSO₄) and filtered. Removal of solvents in vacuum followed by column chromatography [hexanes-ethyl acetate, 5:1 (v/v)] gave Compound 85B as colourless oil (74 mg, 56%).

### Step C:

To a solution of Compound 85B (65 mg, 0.13 mmol) in dichloromethane (5 ml), trifluoroacetic acid (0.3 ml) was added. The mixture was heated at reflux overnight. After being cooled to r.t., solvents were removed in vacuum. Ethyl acetate was added and the mixture was quenched with saturated sodium bicarbonate solution. Layers were separated and the aqueous layer was extracted with dichloromethane (2×50 mL), dried (MgSO₄) and filtered. Removal of solvents in vacuum gave Compound 88C yellow oil. Compound 85C was used in the next step without further purification.

### Step D:

To a solution of Compound 85C (Step 3) in methanol (5 ml) at 0° C., ammonia was purged through the solution for 20 min. The mixture was then heated in a sealed-tube at 60° C. for 2 days. After being cooled to r.t., the solid was filtered to give Example 85 as white solid (22 mg, 55%). LCMS: MH⁺=302.

### Example 86

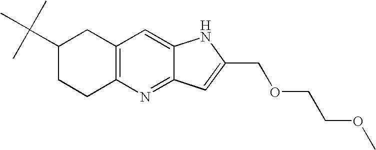

### Preparative Example 86

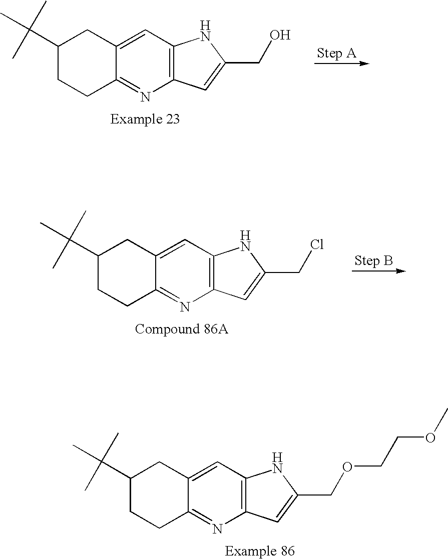

### Step A:

Example 23 (289 mg, 1.12 mmol) was dissolved in phosphorous oxychloride (1.6 ml) and the mixture was stirred at r.t. for 4 hr. Ethyl acetate was added. The mixture was quenched by added to a mixture of ice/water carefully. Layers were separated and the organic layer was washed with water (2×100 mL), dried (MgSO₄) and filtered. Removal of solvents in vacuum gave chloride Compound 86A as yellow solid. Compound 86A was used in the next step without further purification.

### Step B:

To a solution of Compound 86A (50 mg, 0.18 mmol) in 2-methoxyethanol (1 ml) at r.t., potassium carbonate (50 mg, 0.36 mmol) was added. The mixture was stirred at 100° C. for 4 hr. After being cooled to r.t., the mixture was diluted with ethyl acetate and water. Layers were separated and the organic layer was washed with water, dried (MgSO₄) and filtered. Removal of solvents in vacuum followed by column chromatography (ethyl acetate) gave Example 86 as white solid (37 mg, 65%). LCMS: MH⁺=317.

### Example 87

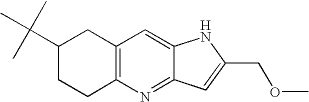

Example 87 was prepared from Compound 86A using the similar procedure as described for the preparation of Example 86 from Compound 86A but using methanol in place of 2-methoxyethanol. LCMS: MH⁺=273.

### Example 88

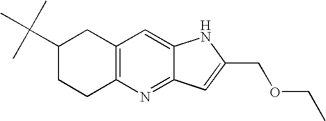

Example 88 was prepared from Compound 86A using the similar procedure as described for the preparation of Example 86 from Compound 86A but using ethanol in place of 2-methoxyethanol. LCMS MH⁺=287.

### Example 89

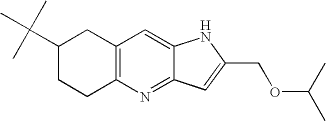

Example 89 was prepared from Compound 86A using the similar procedure as described for the preparation of Example 86 from Compound 86A but using isopropanol in place of 2-methoxyethanol. LCMS: MH⁺=301.

### Example 90

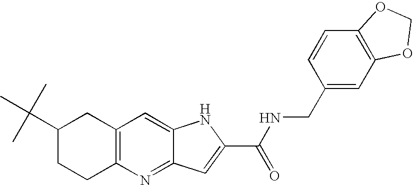

### Preparative Example 90

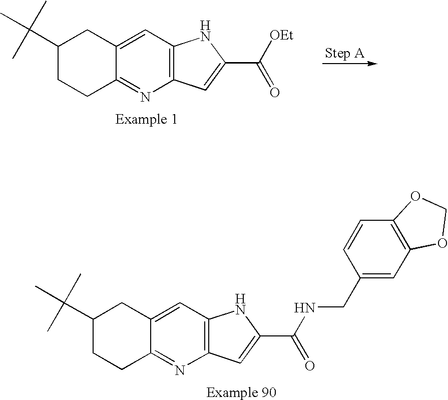

### Step A:

A solution of Example 1 (50 mg, 0.17 mmol), amine (0.5 ml) and a catalytic amount of sodium cyanide were heated in a sealed-tube at 120° C. overnight. After being cooled to r.t., the mixture was diluted with water and ethyl acetate. The organic layer was washed with water (100×2), dried (MgSO₄) and filtered. Removal of solvents in vacuum gave white solid. The solid was washed with ether to give Example 90 as white solid (28 mg, 40%). LCMS: MH⁺=406.

### Example 91

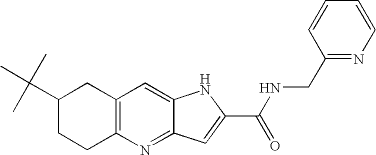

The Example 91 was prepared from Example 1 using the same procedure as described for the preparation of the Example 90 from Example 1. Example 91 was obtained as yellow solid (31 mg, 50%). LCMS: MH⁺=363.

### Example 92

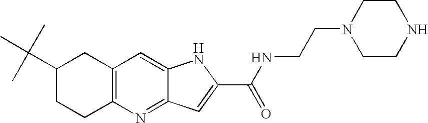

The Example 92 was prepared from Example 1 using the same procedure as described for the preparation of the Example 90 from Example 1. Example 92 was obtained as white solid (32 mg, 40%). LCMS: MH⁺=384.

### Example 93

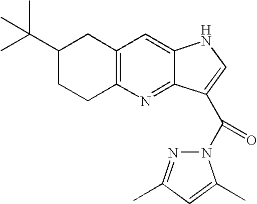

### Preparative Example 93

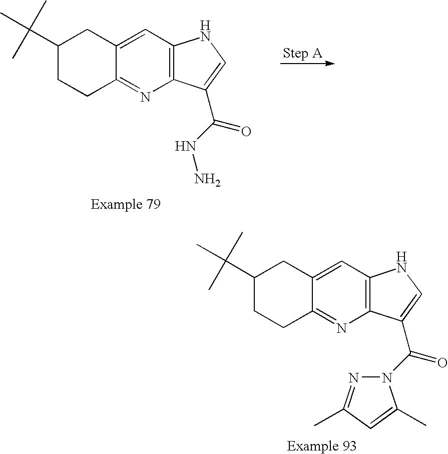

### Step A:

To a solution of Example 79 (150 mg, 0.52 mmol) in tetrahydrofuran (5 ml), 2,4-pentanedione (108 μl, 1.05 mmol) and 1 drop of concentrated hydrochloric acid were added. The mixture was stirred at r.t. for 1 hr. Ethyl acetate and water were added. The mixture was quenched with saturated sodium bicarbonate solution. Layers were separated and the aqueous layer was extracted with ethyl acetate (2×100 mL), dried (MgSO₄) and filtered. Removal of solvents in vacuum gave white solid. The solid was washed with ether to give Example 93 as white solid (64 mg, 35%). LCMS: MH⁺=351.

### Example 94

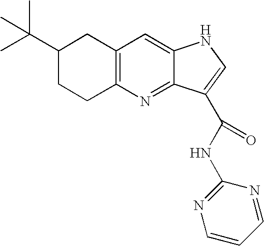

### Preparative Example 94

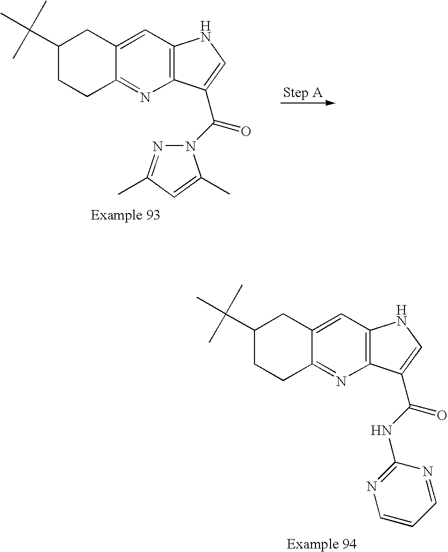

### Step A:

A mixture of Example 93 (65 mg, 0.19 mmol) and 2-aminopyrimidine (180 mg, 1.9 mmol) in acetonitrile (2 ml) was heated in microwave (10 min., 150° C.). Solid was filtered and washed with methanol to give Example 94 as white solid (7 mg, 10%). LCMS: MH⁺=350.

### Example 95

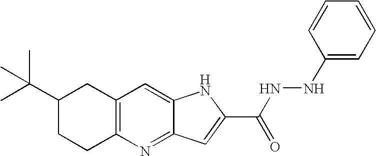

### Preparative Example 95

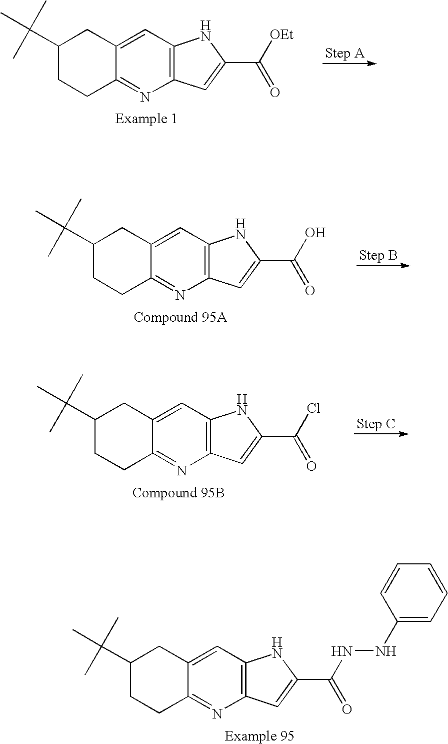

### Step A:

Example 1 (1.5 g, 4.99 mmol) and lithium hydroxide (240 mg, 10 mmol) were stirred in a mixture of water/methanol/tetrahydrofuran (1:1:1 v/v) at reflux for 1 hr. After being cooled to r.t., solvents were removed in vacuum. The mixture was diluted with water and conc. Hydrochloric acid was added until solution pH=3. The solid was filtered, washed with water and dried under vacuum to give Compound 95A as white solid (1.2 g, 90%).

### Step B:

Compound 95A (55 mg, 0.20 mmol) was dissolved in a mixture of thionyl chloride (2.5 ml) and dichloromethane (2.5 ml). A catalytic amount of DMF (1 drop) was added and the mixture was stirred at r.t. for 15 min. and solvents were removed in vacuum to give Compound 95B as yellow solid. Compound 95B was used in the next step without further purification.

### Step C:

To a suspension of Compound 95B (Step 2) in tetrahydrofuran (10 ml) at r.t., excess phenylhydrazine (2-4 eq.) was added and the mixture was stirred at r.t. overnight. Solvents were removed in vacuum followed by column chromatography [methanol-dichloromethane, 5:95 (v/v)] gave Example 95 as yellow solid (29 mg, 40%). LCMS: MH⁺=363.

### Example 96

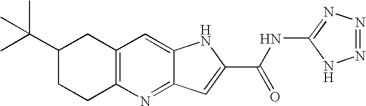

The Example 96 was prepared from Example 1 using the same procedure as described for the preparation of the Example 95 from Example 1. Example 96 was obtained as white solid (5 mg, 9%). LCMS: MH⁺=340.

### Example 97

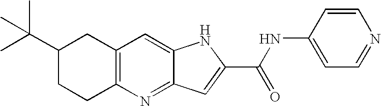

The Example 97 was prepared from Example 1 using the same procedure as described for the preparation of the Example 95 from Example 1. Example 97 was obtained as yellow solid (31 mg, 50%). LCMS: MH⁺=349.

### Example 98

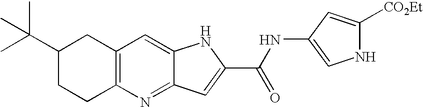

The Example 98 was prepared from Example 1 using the same procedure as described for the preparation of the Example 95 from Example 1. Example 98 was obtained as white solid (26 mg, 35%). LCMS: MH⁺=409.

### Example 99

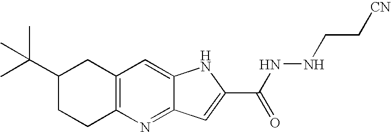

The Example 99 was prepared from Example 1 using the same procedure as described for the preparation of the Example 95 from Example 1. Example 99 was obtained as yellow solid (15 mg, 25%). LCMS: MH⁺=340.

### Example 100

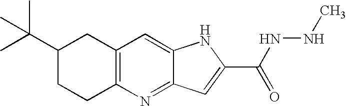

The Example 100 was prepared from Example 1 using the same procedure as described for the preparation of the Example 95 from Example 1. Example 100 was obtained as white solid (24 mg, 45%). LCMS: MH⁺=301.

### Example 101

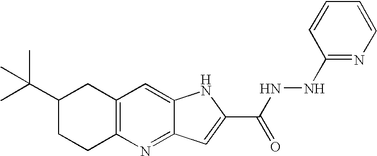

The Example 101 was prepared from Example 1 using the same procedure as described for the preparation of the Example 95 from Example 1. Example 101 was obtained as yellow solid (33 mg, 50%). LCMS: MH⁺=364.

### Example 102

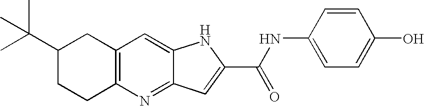

The Example 102 was prepared from Example 1 using the same procedure as described for the preparation of the Example 95 from Example 1. Example 102 was obtained as yellow solid (36 mg, 55%). LCMS: MH⁺=364.

### Example 103A and 103B

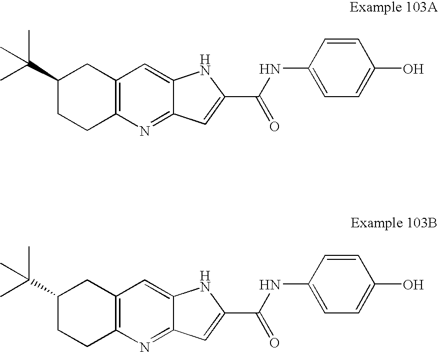

### Preparative Example 103A and 103B

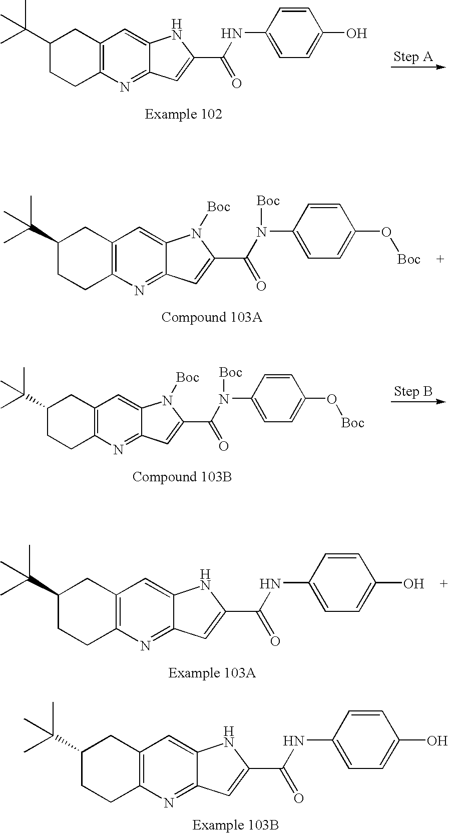

### Step A:

To a solution of Example 102 (310 mg, 1.14 mmol) in tetrahydrofuran (20 ml), (Boc)₂O (1.4 g, 6.3 mmol) followed by triethylamine (0.9 ml, 6.3 mmol) were added. A catalytic amount of DMAP was added and the mixture was stirred at r.t. overnight. The mixture was quenched with saturated sodium bicarbonate solution. Layers were separated and the aqueous layer was extracted with dichloromethane (2×100 mL), dried (MgSO₄) and filtered. Removal of solvents in vacuum followed by column chromatography [hexanes-ethyl acetate, 5:1 (v/v)] gave a mixture of Compound 103A and Compound 103B (334 mg, 80%) as colorless oil.

### Step B:

Chiral HPLC separation of mixture of Compounds 103A and 103B from step 1 [Chiral AD, hexanes-isopropanol, 1:1 (v/v)] first gave the less polar isomer Compound 103B as white foam. [α]_{D}²⁰ −55 (c 0.49, MeOH) and more polar isomer Compound 103A as white foam. [α]_{D}²⁰ +54 (c 0.49, MeOH). Compound 103B was dissolved in dichloromethane (5 ml) and trifluoroacetic acid (5 ml). The mixture was stirred at r.t. for 2 hr and solvents were removed in vacuum to give Example 103B as yellow solid as trifluoroacetic acid salt. LCMS: MH⁺=364.

Compound 103A was dissolved in dichloromethane (5 ml) and trifluoroacetic acid (5 ml). The mixture was stirred at r.t. for 2 hr and solvents were removed in vacuum to give Example 103A as yellow solid as trifluoroacetic acid salt. LCMS: MH⁺=364.

### Example 104

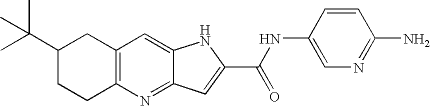

The Example 104 was prepared from Example 1 using the same procedure as described for the preparation of the Example 95 from Example 1. Example 104 was obtained as yellow solid (29 mg, 45%). LCMS: MH⁺=364.

### Example 105

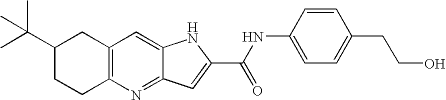

The Example 105 was prepared from Example 1 using the same procedure as described for the preparation of the Example 95 from Example 1. Example 105 was obtained as white solid (86 mg, 55%). LCMS: MH⁺=392.

### Example 106

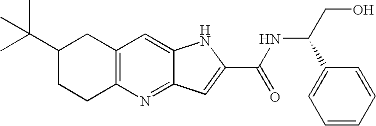

The Example 106 was prepared from Example 1 using the same procedure as described for the preparation of the Example 95 from Example 1. Example 106 was obtained as white solid (35 mg, 50%). LCMS: MH⁺=392.

### Example 107

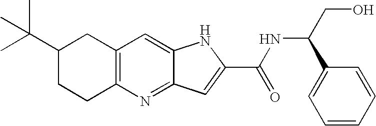

The Example 107 was prepared from Example 1 using the same procedure as described for the preparation of the Example 95 from Example 1. Example 107 was obtained as yellow solid (39 mg, 55%). LCMS: MH⁺=392.

### Example 108

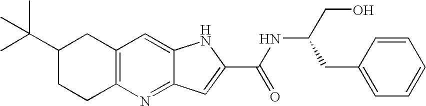

The Example 108 was prepared from Example 1 using the same procedure as described for the preparation of the Example 95 from Example 1. Example 108 was obtained as (33 mg, 45%). LCMS: MH⁺=406.

### Example 109

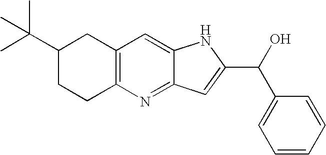

### Preparative Example 109

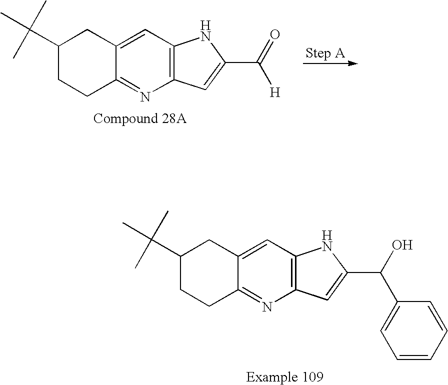

### Step A:

To a solution of Compound 28A (30 mg, 0.12 mmol, Example 107, step 1) in tetrahydrofuran (5 ml) at 0° C., phenylmagnesium bromide (0.12 ml, 0.35 mmol, 3.0M in ether) was added and the mixture was stirred at 0° C. for 15 min. before quenched with saturated ammonium chloride solution. Ethyl acetate and water were added. Layers were separated, dried (MgSO₄) and filtered. Removal of solvents in vacuum followed by column chromatography [hexanes-ethyl acetate, 1:1 (v/v)] gave Example 109 as white solid (25 mg, 65%). LCMS: MH⁺=335.

### Example 110

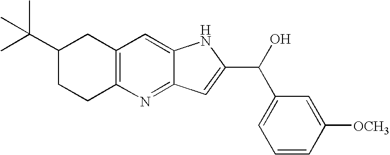

The Example 110 was prepared from Compound 28A using the same procedure as described for the preparation of the Example 109 from Compound 28A. Column chromatography [hexanes-ethyl acetate, 1:1 (v/v)] gave Example 110 as white solid (26 mg, 62%). LCMS: MH⁺=365.

### Example 111

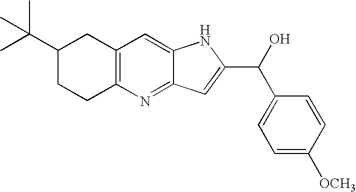

The Example 111 was prepared from Compound 28A using the same procedure as described for the preparation of the Example 109 from Compound 28A. Column chromatography [hexanes-ethyl acetate, 1:1 (v/v)] gave Example 111 as white solid (26 mg, 60%). LCMS: MH⁺=365.

### Example 112

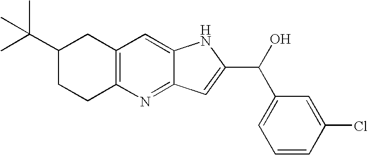

The Example 112 was prepared from Compound 28A using the same procedure as described for the preparation of the Example 109 from Compound 28A. Column chromatography [hexanes-ethyl acetate, 1:1 (v/v)] gave Example 112 as white solid (30 mg, 70%). LCMS: MH⁺=369.

### Example 113

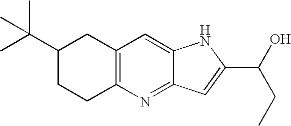

The Example 113 was prepared from Compound 28A using the same procedure as described for the preparation of the Example 109 from Compound 28A. Column chromatography [hexanes-ethyl acetate, 1:1 (v/v)] gave Example 113 as white solid (24 mg, 72%). LCMS: MH⁺=287.

### Example 114

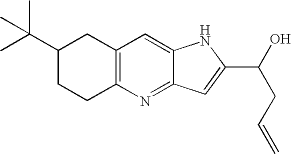

The Example 114 was prepared from Compound 28A using the same procedure as described for the preparation of the Example 109 from Compound 28A. Column chromatography [hexanes-ethyl acetate, 1:1 (v/v)] gave Example 114 as white solid (24 mg, 70%). LCMS: MH⁺=299.

### Example 115

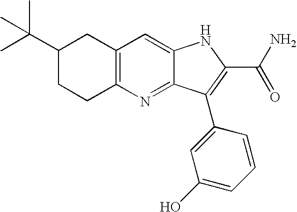

### Preparative Example 115

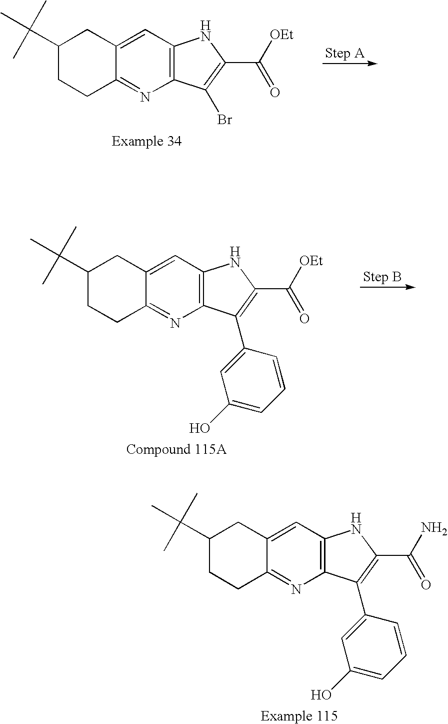

### Step A:

To a mixture of Example 34 (50 mg, 0.13 mmol) and phenylboronic acid (24 mg, 0.17 mmol), toluene (1 ml) and ethanol (1 ml) followed by 2N saturated sodium bicarbonate (0.5 ml) were added. The mixture was purged with nitrogen for 10 min. and palladium tetrakis(triphenyl)phosphine (10% mmol was added. The mixture was heated in a sealed-tube at 90° C. overnight. After being cooled to r.t., ethyl acetate and saturated ammonium chloride solution were added. Layers were separated, dried (MgSO₄) and filtered. Removal of solvents in vacuum followed by column chromatography [hexanes-ethyl acetate, 2:1 (v/v)] gave Compound 115A as white solid (36 mg, 70%).

### Step B:

To a solution of Compound 115A (from above) in methanol (5 ml) at 0° C., ammonia was purged through the solution for 20 min. The mixture was then heated in a sealed-tube at 60-75° C. for 2 days. After being cooled to r.t., the solid was filtered and washed extensively with ether to give Example 115 as white solid (30 mg, 90%). LCMS: MH⁺=364.

### Example 116

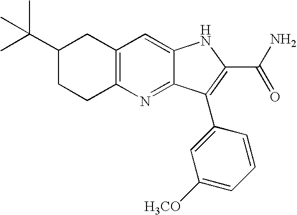

The Example 116 was prepared from Example 34 using the same procedure as described for the preparation of the Example 115 from Example 34. Example 116 was obtained as white solid (32 mg, 63%, 2 steps). LCMS: MH⁺=378.

### Example 117

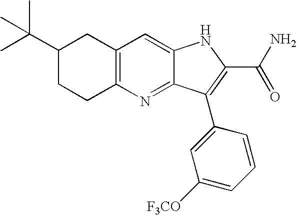

The Example 117 was prepared from Example 34 using the same procedure as described for the preparation of the Example 115 from Example 34. Example 117 was obtained as white solid (33 mg, 59%, 2 steps). LCMS: MH⁺=432.

### Example 118

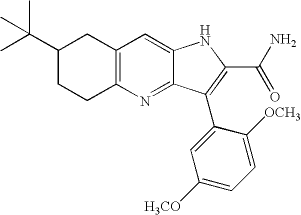

The Example 118 was prepared from Example 34 using the same procedure as described for the preparation of the Example 115 from Example 34. Example 118 was obtained as white solid (31 mg, 59%, 2 steps). LCMS: MH⁺=408.

### Example 119

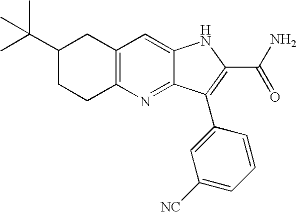

The Example 119 was prepared from Example 34 using the same procedure as described for the preparation of the Example 115 from Example 34. Example 119 was obtained as white solid (27 mg, 54%, 2 steps). LCMS: MH⁺=373.

### Example 120

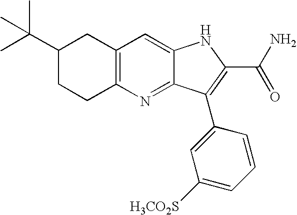

The Example 120 was prepared from Example 34 using the same procedure as described for the preparation of the Example 115 from Example 34. Example 120 was obtained as white solid (33 mg, 59%, 2 steps). LCMS: MH⁺=426.

### Example 121

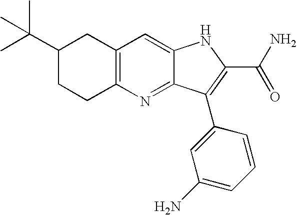

The Example 121 was prepared from Example 34 using the same procedure as described for the preparation of the Example 115 from Example 34. Example 121 was obtained as white solid (22 mg, 45%, 2 steps). LCMS: MH⁺=363.

### Examples 122-124

Examples 122-124 were prepared from compound 95B using the procedures as described for the preparation of similar compounds in the patent # WO 2006098961.

| Example | Structure | LCMS: MH⁺ |
|---|---|---|
| 122 | 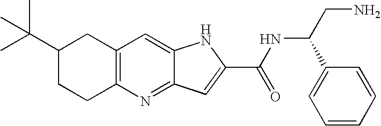 | 391 |
| 123 | 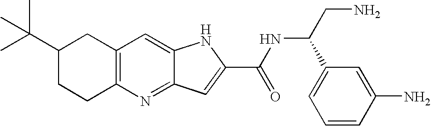 | 406 |
| 124 | 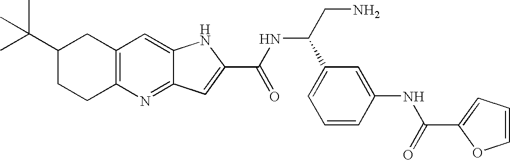 | 500 |

Numerical IC₅₀ values for some of the representative compounds in Table 2 below:

TABLE 2

| | | IC₅₀ |
|---|---|---|
| Example | Structure | (μM) |
| 2 | 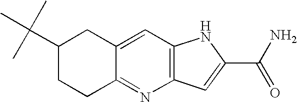 | 0.03 |
| 3 | 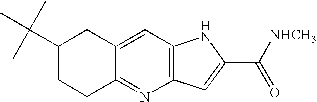 | 0.05 |
| 21A | 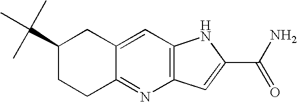 | 0.03 |
| 22A | 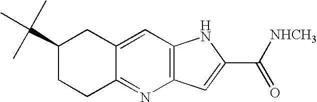 | 0.02 |
| 23 | 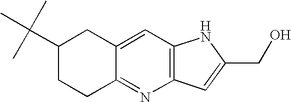 | 0.04 |
| 24 | 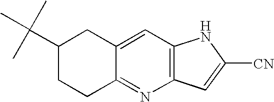 | 0.05 |

### REFERENCES

### KSP/Kinesin as Target

1) Blangy, A et al. (1995) Cell 83, 1159-1169 (cloning of human KSP, function in mitosis).
2) Sawin, K. and Mitchison, T. J. (1995) Proc. Natl. Acad. Sci. 92, 4289-4293 (Xenopus Egd5, conserved motor domain, function).
3) Huang, T.-G. and Hackney, D. D. (1994) J. Biol. Chem. 269, 16493-16501 (Drosphila kinesin minimal motor domain definition, expression and purification from E. coli).
4) Kaiser A. et al. (1999) J. Biol. Chem. 274, 18925-18931 (overexpression of KSP motor domain, function in mitosis, inhibition of growth by targeting KSP).
5) Kapoor T. M and Mitchison, T. J. (1999) Proc. Natl. Acad. Sci. 96, 9106-9111 (use of KSP motor domain, inhibitors thereof).
6) Mayer, T. U. (1999) Science 286, 971-974 (KSP inhibitors as anticancer drugs).

### KSP Assays (Endpoint and Kinetics)

7) Wohlke, G. et al. (1997) Cell 90, 207-216 (expression and purification of kinesin motor domain, kinetics assay, endpoint assay).
8) Geladeopoulos, T. P. et al. (1991) Anal. Biochem. 192, 112-116 (basis for endpoint assay).
9) Sakowicz, R. et al. (1998) Science 280, 292-295 (kinetics assay).
10) Hopkins, S. C. et al. (2000) Biochemistry 39, 2805-2814 (endpoint and kinetics assay).
11) Maliga, Z. et al. (2002) Chem. & Biol. 9, 989-996 (kinetics assay).

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. A compound represented by the structural Formula (I): 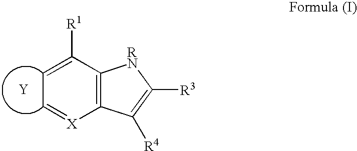
or a pharmaceutically acceptable salt, solvate or ester thereof, wherein:
ring Y is a 3- to 7-membered cycloalkyl or cycloalkenyl fused as shown in Formula I, wherein each of said 3- to 7-membered cycloalkyl or cycloalkenyl, is optionally substituted with 1-2 R² moieties;
X is N or N-oxide;
R and R¹ are each independently selected from the group consisting of selected from the group consisting of H, halo, alkyl, cycloalkyl, cycloalkylalkyl, heterocyclyl, heterocyclylalkyl, aryl, aralkyl, heteroaryl, heteroaralkyl, —(CR¹¹R¹²)₀₋₆—OR⁸, —C(O)R⁵, —C(S)R⁵, —C(O)OR⁸, —C(S)OR⁸, —OC(O)R⁸, —OC(S)R⁸, —C(O)NR⁵R⁶, —C(S)NR⁵R⁶, —C(O)NR⁵OR⁸, —C(S)NR⁵OR⁸, —C(O)NR⁸NR⁵R⁶, —C(S)NR⁸NR⁵R⁸, —C(S)NR⁵OR⁸, —C(O)SR⁸, —NR⁵R⁶, —NR⁵C(O)R⁶, —NR⁵C(S)R⁶, —NR⁵C(O)OR⁸, —NR⁵C(S)OR⁸, —OC(O)NR⁵R⁶, —OC(S)NR⁵R⁶, —NR⁵C(O)NR⁵R⁶, —NR⁵C(S)NR⁵R⁶, —NR⁵C(O)NR⁵OR⁸, —NR⁵C(S)NR⁵OR⁸, —(CR¹¹R¹²)₀₋₆SR⁸, SO₂R⁸, —S(O)₁₋₂NR⁵R⁶, —N(R⁸)SO₂R⁸, —S(O)₁₋₂NR⁶OR⁸, —CM, —OCF₃, —C(═NR⁸)NR⁵, —C(O)NR⁸(CH₂)₁₋₁₀NR⁵R⁶, —C(O)NR⁸(CH₂)₁₋₁₀OR⁶, —C(S)NR⁸(CH₂)₁₋₁₀NR⁵R⁶, —C(S)NR⁸(CH₂)₁₋₁₀OR⁸, haloalkyl and alkylsilyl, wherein each of said alkyl, cycloalkyl, cycloalkylalkyl, heterocyclyl, heterocyclylalkyl, aryl, aralkyl, heteroaryl or heteroaralkyl is independently optionally substituted with 1-5 R¹⁰ moieties;
each R² is independently selected from the group consisting of H, halo, alkyl, cycloalkyl, alkylsilyl, cycloalkenyl, heterocyclyl, heterocyclenyl, aryl, heteroaryl, —(CR¹¹R¹²)₀₋₆—OR⁸, —C(O)R⁵, —C(S)R⁵, —C(O)OR⁸, —C(S)OR⁸, —OC(O)R⁸, —OC(S)R⁸, —C(O)NR⁵R⁶, —C(S)NR⁵R⁸, —C(O)NR⁵OR⁸, —C(S)NR⁵OR⁸, —C(O)NR⁸NR⁵R⁶, —C(S)NR⁸NR⁵R⁶, —C(S)NR⁵OR⁸, —C(O)SR⁸, —NR⁵R⁶, —NR⁵C(O)R⁶, —NR⁵C(S)R⁶, —NR⁵C(O)OR⁸, —NR⁵C(S)OR⁸, —OC(O)NR⁵R⁶, —OC(S)NR⁵R⁶, —NR⁵C(O)NR⁵R⁶, —NR⁵C(S)NR⁵R⁶, —NR⁵C(O)NR⁵OR⁸, —NR⁵C(S)NR⁵OR⁸, —(CR¹¹R¹²)₀₋₆SR⁸, SO₂R⁸, —S(O)₁₋₂NR⁵R⁶, —N(R⁸)SO₂R⁸, —S(O)₁₋₂NR⁶OR⁸, —CN, —OCF₃, —SCF₃, —C(═NR⁸)NR⁵, —C(O)NR⁸(CH₂)₁₋₁₀NR⁵R⁶, —C(O)NR⁸(CH₂)₁₋₁₀OR⁸, —C(S)NR⁸(CH₂)₁₋₁₀NR⁵R⁶, and —C(S)NR⁸(CH₂)₁₋₁₀OR⁸, wherein each of said alkyl, cycloalkyl, cycloalkenyl, heterocyclyl, heterocyclenyl, aryl, and heteroaryl is independently optionally substituted with 1-5 R¹⁰ moieties;
or two R²s on the same carbon atom are optionally taken together with the carbon atom to which they are attached to form a C═O, a C═S or an ethylenedioxy group;
R³ and R⁴ are each independently selected from the group consisting of H, halo, hydroxy, nitro, alkyl, alkenyl, alkynyl, alkoxy, cycloalkyl, cycloalkenyl, heterocyclyl, heterocyclenyl, aryl, heteroaryl, —C(O)R⁵, —C(S)R⁵, —C(O)OR⁸, C(S)OR⁸, —OC(O)R⁸, —OC(S)R⁶, —C(O)NR⁵R⁶, —C(S)NR⁵R⁶, —C(O)NR⁵OR⁸, —C(S)NR⁵OR⁸, —C(O)NR⁸NR⁵R⁶, —C(S)NR⁸NR⁵R⁶, —C(S)NR⁵OR⁸, —C(O)SR⁸, —NR⁵R⁶, —NR⁵C(O)R⁶, —NR⁵C(S)R⁶, —NR⁵C(O)OR⁸, —NR⁵C(S)OR⁸, —OC(O)NR⁵R⁶, —OC(S)NR⁵R⁶, —NR⁵C(O)NR⁵R⁶, —NR⁵C(S)NR⁵R⁶, —NR⁵C(O)NR⁵OR⁸, —NR⁵C(S)NR⁵OR⁸, —(CR¹¹R¹²)₀₋₆SR⁸, SO₂R⁸, —S(O)₁₋₂NR⁵R⁶, —N(R⁸)SO₂R⁸, —S(O)₁₋₂NR⁶OR⁸, —CN, —C(═NR³)NR⁵R⁸, —C(═NOR⁸)R⁵, —C═N—N(R⁸)—C(═S)NR⁵R⁶, —C(O)N(R⁸)—(CR⁴⁰R⁴¹)₁₋₅—C(═NR⁸)NR⁵R⁶, C(O)N(R⁸)(CR⁴⁰R⁴¹)₁₋₅—NR⁵R⁸, —C(O)N(R⁸)(CR⁴⁰R⁴¹)₁₋₅—C(O)—NR⁵R⁶, —C(O)N(R⁸)(CR⁴⁰R⁴¹)₁₋₅—OR⁸, —C(S)NR⁸(CH₂)₁₋₅NR⁵R⁶, and —C(S)NR⁸(CH₂)₁₋₅ OR⁸, wherein each of said alkyl, alkenyl, alkynyl, cycloalkyl, cycloalkenyl, heterocyclyl, heterocyclenyl, aryl, and heteroaryl is independently optionally substituted with 1-5 R¹⁰ moieties;
each of R⁵ and R⁶ is independently selected from the group consisting of H, alkyl, alkenyl, alkynyl, cycloalkyl, cycloalkenyl, heterocyclyl, heterocyclenyl, aryl, heteroaryl, —OR⁸, —C(O)R⁸, and —C(O)OR⁸, with the proviso that R⁵ and R⁶ are not simultaneously —OR⁸; wherein each of said alkyl, alkenyl, alkynyl, cycloalkyl, cycloalkenyl, heterocyclyl, heterocyclenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl;
each R⁸ is independently selected from the group consisting of H, alkyl, aryl, aralkyl, cycloalkyl, cycloalkylalkyl, heterocyclyl, heterocyclylalkyl, heteroaryl, and heteroaralkyl, wherein each member of R⁸ except H is optionally substituted with 1-4 R⁹ moieties;
each R⁹ is independently selected from the group consisting of halo, alkyl, cycloalkyl, cycloalkenyl, heterocyclyl, heterocyclenyl, aryl, heteroaryl, —NO₂, —OR¹¹, —OC(═O)R¹¹, —(C₁-C₆ alkyl)-OR¹¹, —CN, —NR¹¹R¹², —C(O)R¹¹, —C(O)OR¹¹, —C(O)NR¹¹R¹², —CF₃, —OCF₃, CF₂CF₃, C(═NOH)R¹¹, —NR¹¹C(═O)R¹²—C(═NR¹¹)NR¹¹R¹², and —NR¹¹C(═O)OR¹²; wherein said each of said alkyl, cycloalkyl, cycloalkenyl, heterocyclyl, heterocyclenyl, aryl, and heteroaryl is independently optionally substituted with 1-4 R⁴² moieties; wherein when each of said cycloalkyl, cycloalkenyl, heterocyclyl, heterocyclenyl, aryl, and heteroaryl contains two radicals on adjacent carbon atoms anywhere within said cycloalkyl, cycloalkenyl, heterocyclyl, heterocyclenyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl; or two R⁹ groups, when attached to the same carbon, are optionally taken together with the carbon atom to which they are attached to form a C═O or a C═S group;
each R¹⁰ is independently selected from the group consisting of H, alkyl, heterocyclyl, aryl, alkoxy, OH, CN, halo, —(CR¹¹R¹²)₀₋₄NR⁵R⁶, haloalkyl, haloalkoxy, hydroxyalkyl, alkoxyalkyl, —O-alkyl-O-alkyl, —C(O)NR⁵R⁶, —C(O)OR⁸, —OC(O)R⁵, —OC(O)NR⁵R⁶, —NR⁵C(O)R⁶, —NR⁵C(O)OR⁶, —NR⁵C(O)NR⁵R⁶, —SR⁸, —S(O)R⁸, and —S(O)₂R⁸, wherein each of said alky, heterocyclyl and aryl is optionally independently substituted with 1-4 R¹³ moieties;
each R¹¹ is independently H or alkyl;
each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹³ is independently selected from the group consisting of H, halo, alkyl, alkylsilyl, alkoxy, haloalkyl, cyano, and hydroxy;
each R⁴² is independently selected from the group consisting of halo, alkyl, cycloalkyl, heterocyclyl, aryl, heteroaryl, —NO₂, —OR¹¹, —(C₁-C₆ alkyl)-OR¹¹, —CN, —NR¹¹R¹², —C(O)R¹¹, —C(O)OR¹¹, —C(O)NR¹¹R¹², —CF₃, —OCF₃, —N(R¹¹)C(O)R¹², and —NR¹¹C(O)OR¹², wherein each of said aryl, heterocyclyl and heteroaryl is optionally substituted with 1-4 R⁴³ moieties; and
each R⁴³ is independently selected from the group consisting of halo, alkyl, alkoxy, haloalkyl, cyano, and hydroxyl;
with the proviso that R and R³ are optionally taken together, with the ring nitrogen and carbon atom to which they are respectively shown attached, to form a heteroaryl, heterocyclyl or heterocyclenyl ring that is optionally substituted with 1-3 moieties independently selected from the group consisting of oxo, thioxo, —OR¹², —NR¹¹R¹², —C(═O)R¹²—C(═O)OR¹², —C(═O)NR¹¹R¹², and —NR¹¹C(═O)R¹².

2. The compound according to claim 1, wherein ring Y is a 3- to 7-membered cycloalkyl which is optionally substituted with 1-2 R² moieties.

3. The compound according to claim 1, wherein ring Y is a 6-membered cycloalkyl, which is optionally substituted with 1-2 R² moieties.

4. The compound according to claim 1, wherein ring Y is substituted with one R² moiety.

5. The compound according to claim 1, wherein R² is alkyl.

6. The compound according to claim 5, wherein R² is t-butyl.

7. The compound according to claim 1, wherein R is selected from the group consisting of H and —C(O)R⁵.

8. The compound according to claim 7, wherein R⁵ is alkyl.

9. The compound according to claim 1, wherein R¹ is H.

10. The compound according to claim 9, wherein R is H.

11. The compound according to claim 1, wherein:
R³ and R⁴ are each independently selected from the group consisting of H, halo, hydroxy, nitro, alkyl, alkenyl, alkynyl, alkoxy, heterocyclyl, aryl, heteroaryl, —C(O)R⁵, —C(O)OR⁸, —C(O)NR⁵R⁶, —C(O)NR⁸NR⁵R⁶, —NR⁵R⁶, —NR⁵C(O)R⁶, —N(R⁸)SO₂R⁸, —CN, —C(═NOR⁸)R⁵, and —C═N—N(R⁸)—C(═S)NR⁵R⁶, wherein each of said alkyl, alkenyl, alkynyl, heterocyclyl, and aryl is independently optionally substituted with 1-5 R¹⁰ moieties;
each of R⁵ and R⁶ is independently selected from the group consisting of H, alkyl, alkenyl, aryl, heterocyclyl, and heteroaryl wherein each of said alkyl, alkenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;
each R³ is independently alkyl, which is optionally substituted with 1-4 R⁹ moieties;
each R⁹ is independently selected from the group consisting of alkyl, heterocyclyl, aryl, heteroaryl, —OR¹¹, —OC(═O)R¹¹, —CN, —NR¹¹R¹², —NR¹¹C(═O)OR¹², —C(═O)NR¹¹R¹², —NR¹¹C(═O)R¹², and —C(O)OR¹¹; wherein each of said alkyl, heterocyclyl, aryl, and heteroaryl is independently optionally substituted with 1-4 R⁴² moieties; wherein when each of said heterocyclyl, aryl, and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;
each R¹⁰ is independently selected from the group consisting of H, alkyl, alkoxy, OH, CN, halo, heterocyclyl, aryl, heteroaryl, —O-alkyl-O-alkyl, —NR⁵R⁶, haloalkyl, haloalkoxy, hydroxyalkyl, alkoxyalkyl, —C(═O)NR⁵R⁶, —C(═O)OR⁸, OC(═O)R⁵, —OC(═O)NR⁵R⁶, —NR⁵C(═O)R⁶, —NR⁵C(═O)OR⁶, —NR⁵C(═O)NR⁵R⁶, and —S(═O)₂R⁸, wherein each of said heterocyclyl, aryl, and heteroaryl moieties is optionally independently substituted with 1-4 R¹³ moieties;
each R¹¹ is independently H or alkyl; and
each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹³ is independently selected from the group consisting of halo, alkyl, alkoxy, haloalkyl, cyano, and hydroxy;
each R⁴² is independently selected from the group consisting of halo, alkyl, cycloalkyl, heterocyclyl, aryl, heteroaryl, —NO₂, —OR¹¹, —(C₁-C₆ alkyl)-OR¹¹, —ON, —NR¹¹R¹², —C(═O)R¹¹, —C(═O)OR¹¹, —C(═O)NR¹¹R¹², —CF₃, —OCF₃, —NR¹¹C(═O)R¹², and —NR¹¹C(═O)OR¹², wherein each of said aryl, heterocyclyl and heteroaryl is optionally substituted with 1-4 R⁴³ moieties; and
each R⁴³ is independently selected from the group consisting of halo, alkyl, alkoxy, haloalkyl, cyano, and hydroxyl.

12. The compound according to claim 11, wherein:
R³ is selected from the group consisting of H, halo, hydroxy, nitro, alkyl, alkenyl, alkoxy, —C(O)R⁵, —C(O)OR⁸, —C(O)NR⁵R⁶, —C(O)NR⁸NR⁵R⁶, —CN, —C(═NOR⁸)R⁵, and —C═N—N(R⁶)—C(═S)NR⁵R⁶, wherein each of said alkyl and alkenyl is independently optionally substituted with 1-5 R¹⁰ moieties;
each of R⁵ and R⁶ is independently selected from the group consisting of H, alkyl, alkenyl, aryl, heterocyclyl, and heteroaryl wherein each of said alkyl, alkenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;
each R⁸ is independently alkyl, which is optionally substituted with 1-4 R⁹ moieties;
each R⁹ is independently selected from the group consisting of alkyl, aryl, heteroaryl, —OR¹¹, —OC(═O)R¹¹, —CN, —NR¹¹R¹², and —C(O)OR¹¹; wherein said each of said alkyl, aryl, and heteroaryl is independently optionally substituted with 1-4 R⁴² moieties; wherein when each of said aryl and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;
each R¹⁰ is independently selected from the group consisting of alkoxy, OH, haloalkoxy, heterocyclyl, aryl, —NR⁵R⁶, —CN, —OC(═O)R⁵, and —O-alkyl-β-alkyl, wherein each of said heterocyclyl and aryl is optionally independently substituted with 1-4 R¹³ moieties;
each R¹¹ is independently H or alkyl;
each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹³ is independently selected from the group consisting of halo, alkyl, alkoxy, haloalkyl, cyano, and hydroxyl;
each R⁴² is independently selected from the group consisting of halo, alkyl, cycloalkyl, heterocyclyl, aryl, heteroaryl, —NO₂, —OR¹¹, —(C₁-C₅ alkyl)-OR¹¹, —CN, —NR¹¹R¹², —C(═O)R¹¹, —C(═O)OR¹¹, —C(═O)NR¹¹R¹², —CF₃, —OCF₃, —NR¹¹C(═O)R¹², and —NR¹¹C(═O)OR¹², wherein each of said aryl, heterocyclyl and heteroaryl is optionally substituted with 1-4 R⁴³ moieties; and
each R⁴³ is independently selected from the group consisting of halo, alkyl, alkoxy, haloalkyl, cyano, and hydroxyl.

13. The compound according to claim 12, wherein:
R³ is selected from the group consisting of H, alkyl, alkenyl, halo, hydroxyl, cyano, H₂NNH—C(═O)—, alkyl-NH—NH—(C═O)—, heteroaryl-NH—NH—C(═O)—, aryl-alkyl-, alkoxy, NH₂-alkyl-, NC-alkyl-, aryl-C(═O)—O-alkyl-, alkyl-O—C(═O)—, H₂N—C(═O)—, aryl-NH—NH—C(═O)—, aryl-NH—C(═O)—, heteroaryl-NH—C(═O)—, alkyl-C(═O)—, alkyl-NH—C(═O)—, aryl-alkyl-NH—C(═O)—, HO-alkyl-aryl-NH—C(═O)—, heteroaryl-alkyl-NH—C(═O)—, heterocyclyl-alkyl-NH—C(═O)—, H₂N-alkyl-NH—C(═O)—, HO-alkyl-NH—C(═O)—, alkyl-O-alkyl-, NC-alkyl-NH—NH—C(═O)—, alkyl-O-alkyl-O-alkyl-, H₂N—C(═S)—NH—N═CH—, alkyl-C(═NOH)—, and heterocyclyl-C(═O)—; wherein each of said alkyl, alkenyl, and the “alkyl” part of aryl-alkyl- and aryl-alkyl-NH—C(═O)— is optionally substituted with 1-2 moieties selected from the group consisting of hydroxy and NH₂; wherein the “aryl” part of each of said aryl-alkyl-, aryl-NH—C(═O)—, and aryl-alkyl-NH—C(═O)— is optionally substituted with 1-2 moieties selected from the group consisting of halo, alkoxy, hydroxyl, NH₂, and heteroaryl-C(═O)—NH—; and wherein when the “aryl” part of any of said R³ groups contains two adjacent moieties, such moieties have optionally be taken together with the carbon atoms to which they are attached to a form a five to six membered heterocyclyl or heteroaryl.

14. The compound according to claim 11, wherein:
R⁴ is selected from the group consisting of H, halo, nitro, alkyl, alkenyl, alkynyl, heterocyclyl, aryl, —C(═O)R⁵, —C(═O)OR⁸, —C(═O)NR⁵R⁶, —C(═O)NR⁸NR⁵R⁶, —NR⁵R⁶, —NR⁵C(═O)R⁶, —NR⁸SO₂R⁸, wherein each of said alkyl, alkenyl, alkynyl, heterocyclyl, and aryl is independently optionally substituted with 1-5 R¹⁰ moieties;
each of R⁵ and R⁶ is independently selected from the group consisting of H, alkyl, alkenyl, and heteroaryl wherein each of said alkyl, alkenyl, and heteroaryl is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;
each R⁸ is independently alkyl, which is optionally substituted with 1-4 R⁹ moieties;
each R⁹ is independently selected from the group consisting of alkyl, heterocyclyl, aryl, heteroaryl, —OC(═O)R¹¹, —CN, —NR¹¹R¹², —NR¹¹C(═O)OR¹², —C(═O)NR¹¹R¹², —NR¹¹C(═O)R¹², and —C(═O)OR¹¹; wherein said each of said alkyl, heterocyclyl, and heteroaryl is independently optionally substituted with 1-4 R⁴² moieties; wherein when each of said heterocyclyl and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;
each R¹⁰ is independently selected from the group consisting of H, alkyl, alkoxy, OH, CN, —O-alkyl-O-alkyl, —NR⁵R⁶, haloalkoxy, —C(═O)NR⁵R⁶, —NR⁵C(═O)R⁶, —NR⁵C(═O)OR⁶, and —S(═O)₂R⁸;
each R⁴² is independently selected from the group consisting of halo, alkyl, cycloalkyl, heterocyclyl, aryl, heteroaryl, —NO₂, —OR¹¹, —(C₁-C₆ alkyl)-OR¹¹, —ON, —NR¹¹R¹², —C(═O)R¹¹, —C(═O)OR¹¹, —C(═O)NR¹¹R¹², —CF₃, —OCF₃, —N(R¹¹)C(═O)R¹², and —NR¹¹C(═O)OR¹², wherein each of said aryl, heterocyclyl and heteroaryl is optionally substituted with 1-4 R⁴³ moieties;
each R¹¹ is independently H or alkyl; and
each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring having 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl; and
each R⁴³ is independently selected from the group consisting of halo, alkyl, alkoxy, haloalkyl, cyano, and hydroxyl.

15. The compound according to claim 14, wherein R⁴ is selected from the group consisting of H, halo, nitro, H₂N—, alkyl, HO-alkyl-, (HO)₂alkyl-, alkyl-C(═O)-alkyl-C(═O)—NH—, alkenyl-C(═O)-alkyl-C(═O)—NH—, H₂N—C(═O)-alkyl-whose “alkyl” is optionally substituted with an alkyl-C(═O)—NH—, NC-alkyl-, H₂N-alkyl-, alkyl-O—C(═O)—NH—, HO—C(═O)—NH—, alkyl-C(═O)—O-alkyl-C(═O)—NH—, alkyl-O—C(═O)-alkenyl-, heteroaryl-C(═O)—NH—, heterocyclyl, HO-alkynyl-, alkyl-O-alkyl-NH—, HO-alkyl-NH—, alkyl-S(═O)₂NH—, alkyl-O—C(═O)—, HO-alkyl-NH—C(═O)—, (HO)₂alkyl-NH—C(═O)—, H₂N-alkyl-NH—C(═O)—, heterocyclyl-alkyl-NH—C(═O)—, heteroaryl-alkyl-NH—C(═O)—, alkenyl-NH—C(═O)—, H₂N—NH—C(═O)—, H₂N—C(═O)—, alkyl-C(═O)—NH—, heteroaryl-C(═O)—, heteroaryl-NH—C(═O)—, and aryl that is optionally substituted with 1-2 moieties selected from the group consisting of hydroxy, alkoxy, haloalkoxy, cyano, H₂N—, and alkyl-S(═O)—.

16. The compound according to claim 1, wherein;
X is N;
ring Y is a 6-membered cycloalkyl which is substituted with an alkyl;
R is selected from the group consisting of H and —C(O)R⁵;
R¹ is H;
R³ is selected from the group consisting of H, halo, hydroxy, nitro, alkyl, alkenyl, alkoxy, —C(O)R⁵, —C(O)OR⁸, —C(O)NR⁵R⁶, —C(O)NR⁶NR⁵R⁶, —CN, —C(═NOR⁸)R⁵, and —C═N—N(R⁸)—C(═S)NR⁵R⁶, wherein each of said alkyl and alkenyl is independently optionally substituted with 1-5 R¹⁰ moieties; and
R⁴ is selected from the group consisting of H, halo, nitro, alkyl, alkenyl, alkynyl, heterocyclyl, aryl, —C(═O)R⁵, —C(═O)OR⁸, —C(═O)NR⁵R⁶, —C(═O)NR⁸NR⁵R⁶, —NR⁵R⁶, —NR⁵C(═O)R⁶, —NR⁸SO₂R⁸, wherein each of said alkyl, alkenyl, alkynyl, heterocyclyl, and aryl is independently optionally substituted with 1-5 R¹⁰ moieties;
each of R⁵ and R⁶ is independently selected from the group consisting of H, alkyl, alkenyl, aryl, heterocyclyl, and heteroaryl wherein each of said alkyl, alkenyl, aryl, and heteroaryl, is optionally substituted with 1-4 R⁹ moieties; or R⁵ and R⁶, when attached to the same nitrogen atom, are optionally taken together with the nitrogen atom to which they are attached to form a heterocyclyl or heteroaryl, each of which is optionally substituted with 1-4 R⁹ moieties;
each R⁸ is independently alkyl, which is optionally substituted with 1-4 R⁹ moieties;
each R⁹ is independently selected from the group consisting of alkyl, heterocyclyl, aryl, heteroaryl, —OR¹¹, —OC(═O)R¹¹, —CN, —NR¹¹R¹², —NR¹¹C(═O)OR¹², —C(═O)NR¹¹R¹², —NR¹¹C(═O)R¹², and —C(O)OR¹¹; wherein each of said alkyl, heterocyclyl, aryl, and heteroaryl is independently optionally substituted with 1-4 R⁴² moieties; wherein when each of said heterocyclyl, aryl, and heteroaryl contains two radicals on adjacent carbon atoms anywhere within said heterocyclyl, aryl, and heteroaryl, such radicals may optionally and independently in each occurrence, be taken together with the carbon atoms to which they are attached, to form a five- or six-membered cycloalkyl, cycloalkenyl, heterocyclyl, heterocyclenyl, or heteroaryl;
each R¹⁰ is independently selected from the group consisting of H, alkyl, alkoxy, OH, CN, halo, heterocyclyl, aryl, heteroaryl, —O-alkyl-O-alkyl, —NR⁵R⁶, haloalkyl, haloalkoxy, hydroxyalkyl, alkoxyalkyl, —C(═O)NR⁵R⁶, —C(═O)OR⁸, —OC(═O)NR⁵R⁶, —NR⁵C(═O)R⁶, —NR⁵C(═O)OR⁶, —NR⁵C(═O)NR⁵R⁶, and —S(═O)₂R⁸, wherein each of said heterocyclyl, aryl, and heteroaryl moieties is optionally independently substituted with 1-4 R¹³ moieties;
each R¹¹ is independently H or alkyl; and
each R¹² is independently H, alkyl, cycloalkyl, cycloalkenyl, aryl, heterocyclyl, heterocyclenyl, or heteroaryl; or R¹¹ and R¹², when attached to the same nitrogen atom, are optionally taken together with the nitrogen atom to which they are attached to form a 3-6 membered heterocyclic ring haying 0-2 additional heteroatoms selected from N, O or S; wherein each of said R¹² alkyl, cycloalkyl, cycloalkenyl, aryl, heterocyclyl, heterocyclenyl, and heteroaryl is independently optionally substituted with 1-3 moieties selected from the group consisting of —CN, —OH, —NH₂, —N(H)alkyl, —N(alkyl)₂, halo, haloalkyl, CF₃, alkyl, hydroxyalkyl, alkoxy, aryl, aryloxy, and heteroaryl;
each R¹³ is independently selected from the group consisting of halo, alkyl, alkoxy, haloalkyl, cyano, and hydroxy;
each R⁴² is independently selected from the group consisting of halo, alkyl, cycloalkyl, heterocyclyl, aryl, heteroaryl, —NO₂, —OR¹¹, —(C₁-C₆ alkyl)-OR¹¹, —CN, —NR¹¹R¹², —C(═O)R¹¹, —C(═O)OR¹¹, —C(═O)NR¹¹R¹², —CF₃, —OCF₃, —NR¹¹C(═O)R¹², and —NR¹¹C(═O)OR¹², wherein each of said aryl, heterocyclyl and heteroaryl is optionally substituted with 1-4 R⁴³ moieties; and
each R⁴³ is independently selected from the group consisting of halo, alkyl, alkoxy, haloalkyl, cyano, and hydroxyl.

17. The compound according to claim 16, wherein:
X is N;
ring Y is a 6-membered cycloalkyl which is substituted with an alkyl;
R is selected from the group consisting of H and alkyl-C(═O)—:
R¹ is H,
R³ is selected from the group consisting of H, alkyl, alkenyl, halo, hydroxyl, cyano, H₂NNH—C(═O)—, alkyl-NH—NH—(C═O)—, heteroaryl-NH—NH—C(═O)—, aryl-alkyl-, alkoxy, NH₂-alkyl-, NC-alkyl-, aryl-C(═O)—O-alkyl-, alkyl-O—C(═O)—, H₂N—C(═O)—, aryl-NH—NH—C(═O)—, aryl-NH—C(═O)—, heteroaryl-NH—C(═O)—, alkyl-C(═O)—, alkyl-NH—C(═O)—, aryl-alkyl-NH—C(═O)—, HO-alkyl-aryl-NH—C(═O)—, heteroaryl-alkyl-NH—C(═O)—, heterocyclyl-alkyl-NH—C(═O)—, H₂N-alkyl-NH—C(═O)—, HO-alkyl-NH—C(═O)—, alkyl-O-alkyl-, NC-alkyl-NH—NH—C(═O)—, alkyl-H₂N—C(═S)—NH—N═CH—, alkyl-C(═NOH)—, and heterocyclyl-C(═O)—; wherein each of said alkyl, alkenyl, and the “alkyl” part of aryl-alkyl-, aryl-alkyl-NH—C(═O)— is optionally substituted with 1-2 moieties selected from the group consisting of hydroxy and NH₂; wherein the “aryl” part of each of said aryl-alkyl-, aryl-NH—C(═O)—, and aryl-alkyl-NH—C(═O)— is optionally substituted with 1-2 moieties selected from the group consisting of halo, alkoxy, hydroxyl, NH₂, aryl-C(═O)—NH-and heteroaryl-C(═O)—NH—; wherein when the “aryl” part of any of said R³ groups contains two adjacent moieties, such moieties have optionally be taken together with the carbon atoms to which they are attached to a form a five to six membered heterocyclyl or heteroaryl; and
R⁴ is selected from the group consisting of H, halo, nitro, H₂N—, alkyl, HO-alkyl-, (HO)₂alkyl-, alkyl-C(═O)-alkyl-C(═O)—NH—, alkenyl-C(═O)-alkyl-C(═O)—NH—, H₂N—C(═O)-alkyl-whose “alkyl” is optionally substituted with an alkyl-C(═O)—NH—, NC-alkyl-, H₂N-alkyl-, alkyl-O—C(═O)—NH—, HO—C(═O)—NH—, alkyl-C(═O)—O-alkyl-C(═O)—NH—, alkyl-O—C(═O)-alkenyl-, heteroaryl-C(═O)—NH—, heterocyclyl, HO-alkynyl-, alkyl-O-alkyl-NH—, HO-alkyl-NH—, alkyl-S(═O)₂NH—, alkyl-O—C(═O)—, HO-alkyl-NH—C(═O)—, (HO)₂alkyl-NH—C(═O)—, H₂N-alkyl-NH—C(═O)—, heterocyclyl-alkyl-NH—C(═O)—, heteroaryl-alkyl-NH—C(═O)—, alkenyl-NH—C(═O)—, H₂N—NH—C(═O)—, H₂N—C(═O)—, alkyl-C(═O)—NH—, heteroaryl-C(═O)—, aryl-NH—C(═O)—, heteroaryl-NH—C(═O)—, and aryl that is optionally substituted with 1-2 moieties selected from the group consisting of hydroxy, alkoxy, haloalkoxy, cyano, H₂N—, alkyl-S, alkyl-S(═O)—, and alkyl-S(═O)₂—.

18. The compound according to claim 1, selected from the group consisting of: 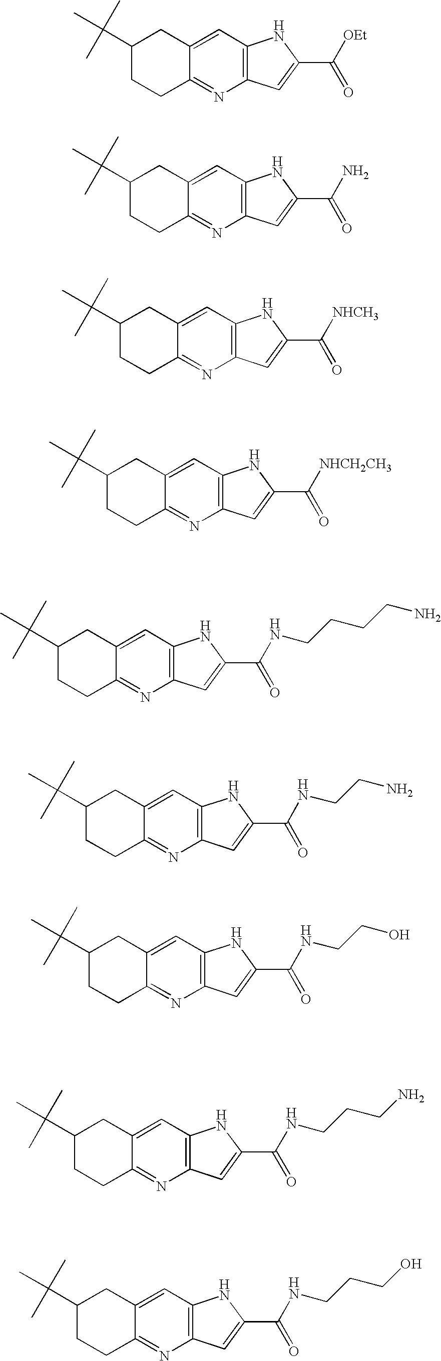 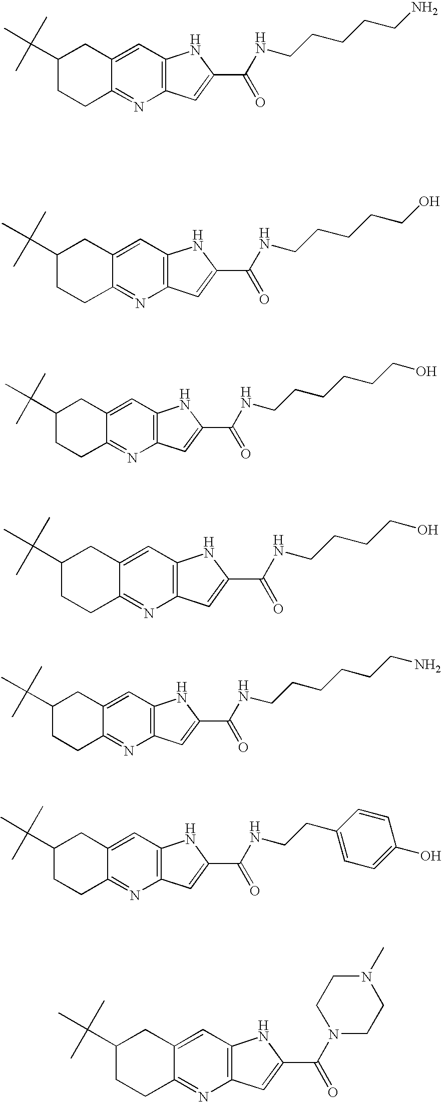 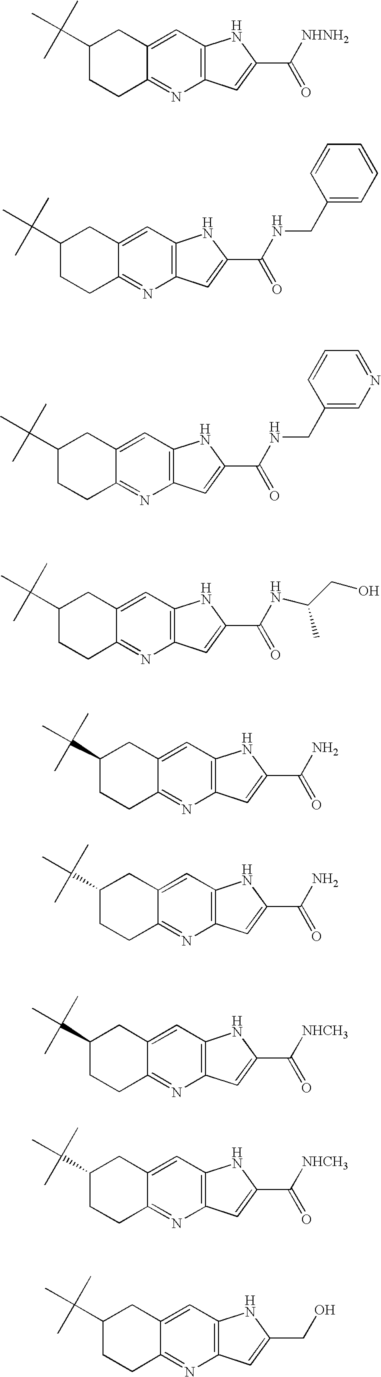 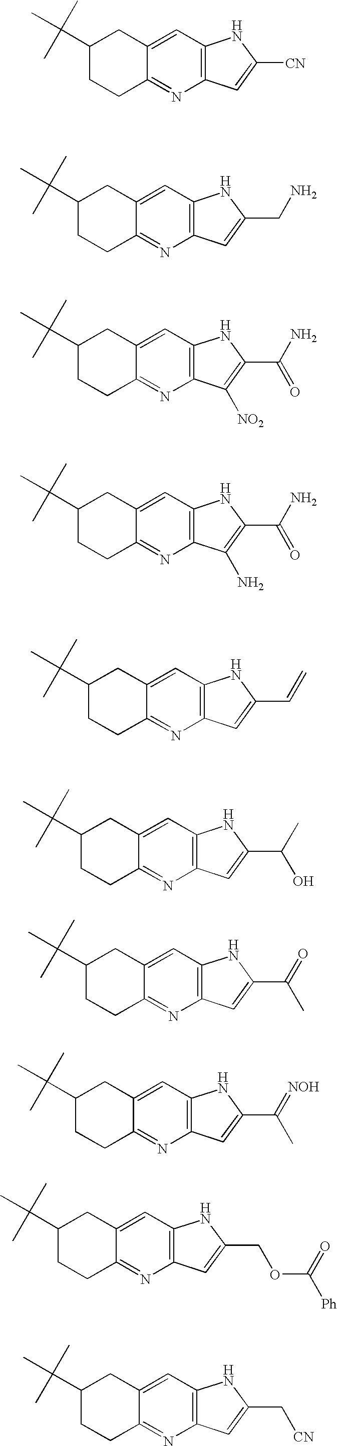 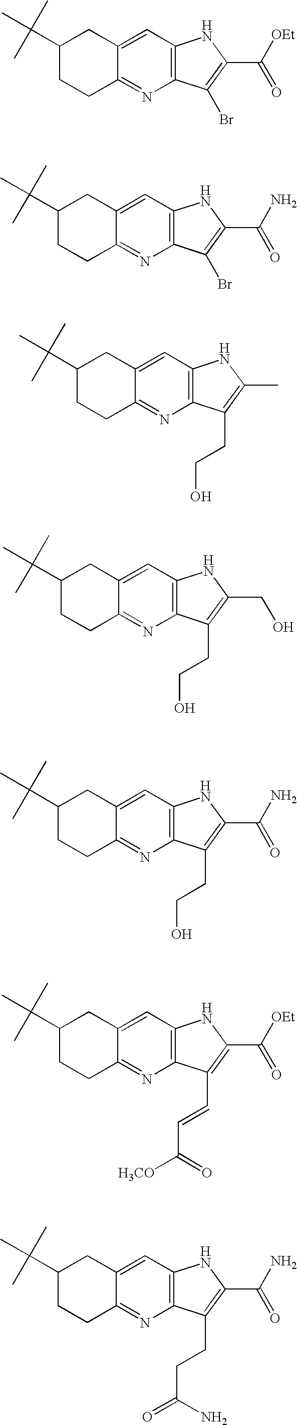 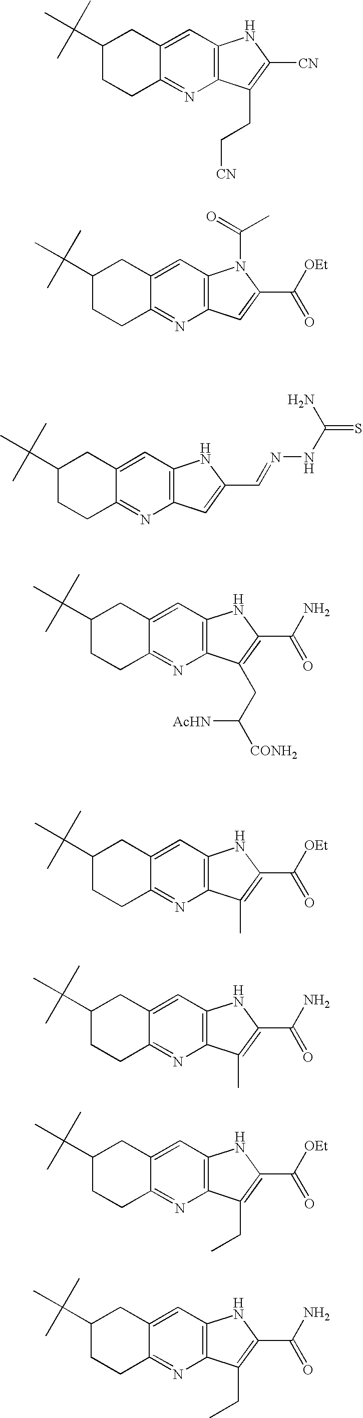 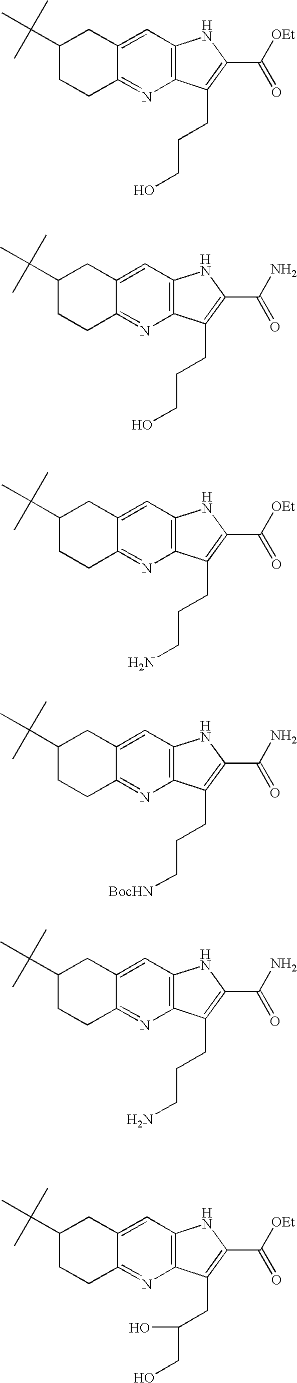 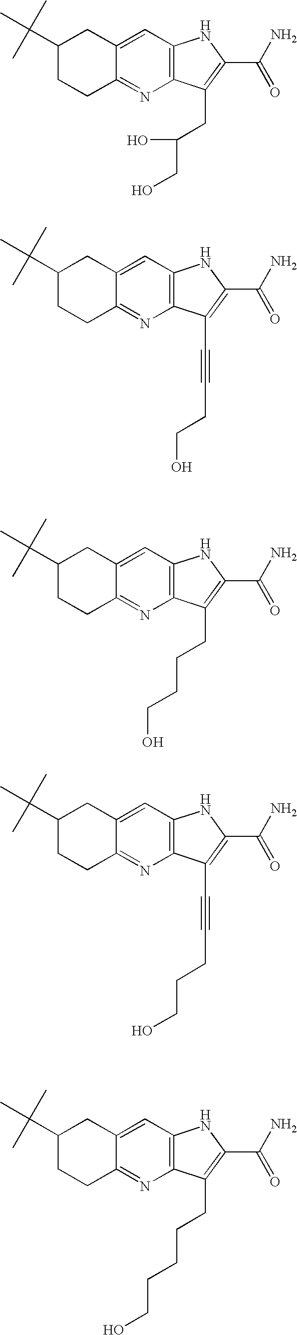 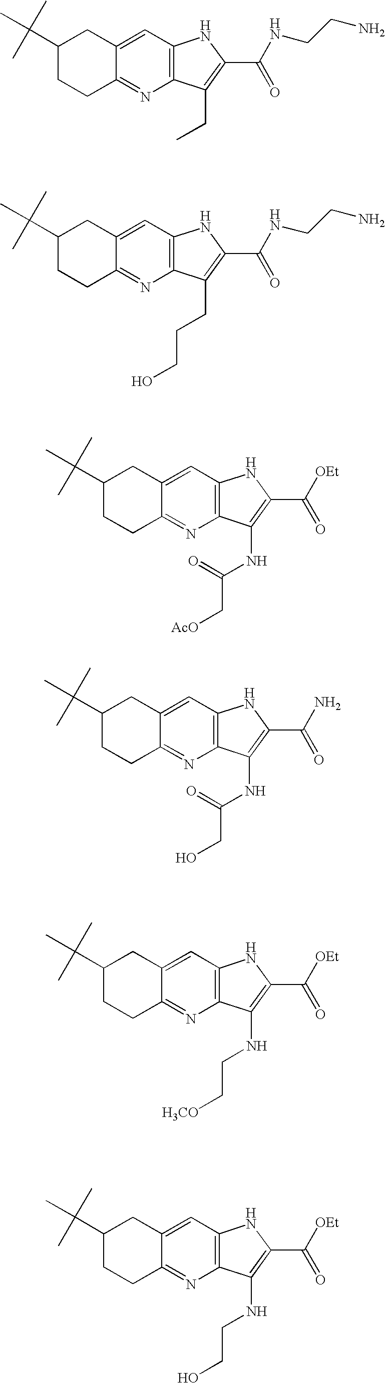 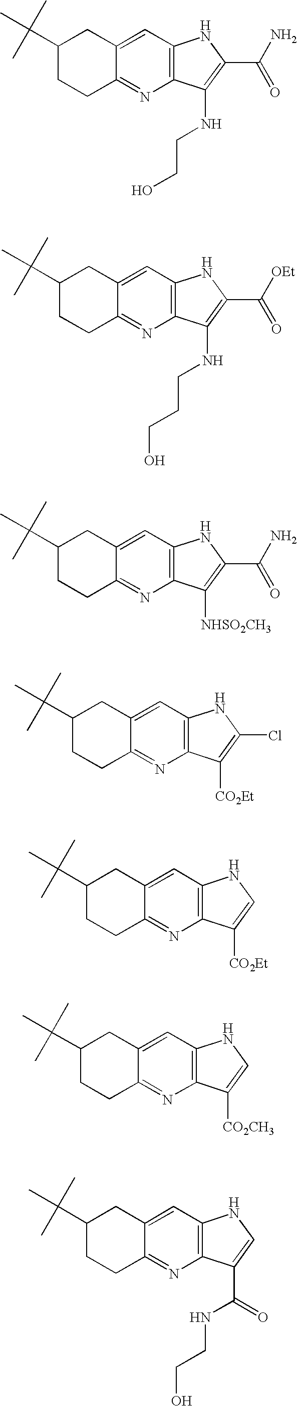 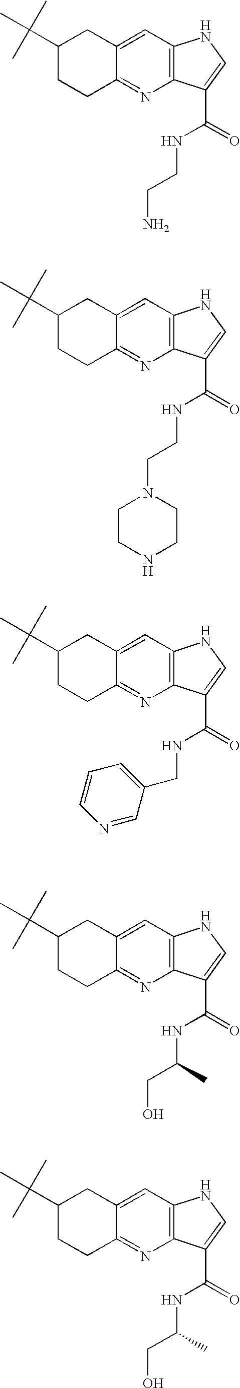 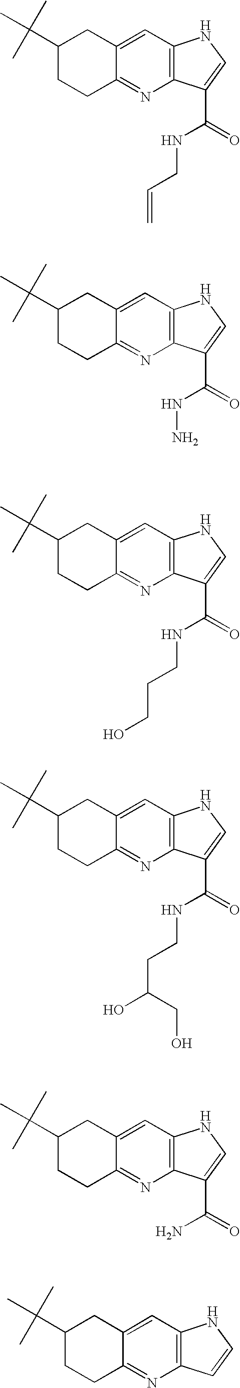 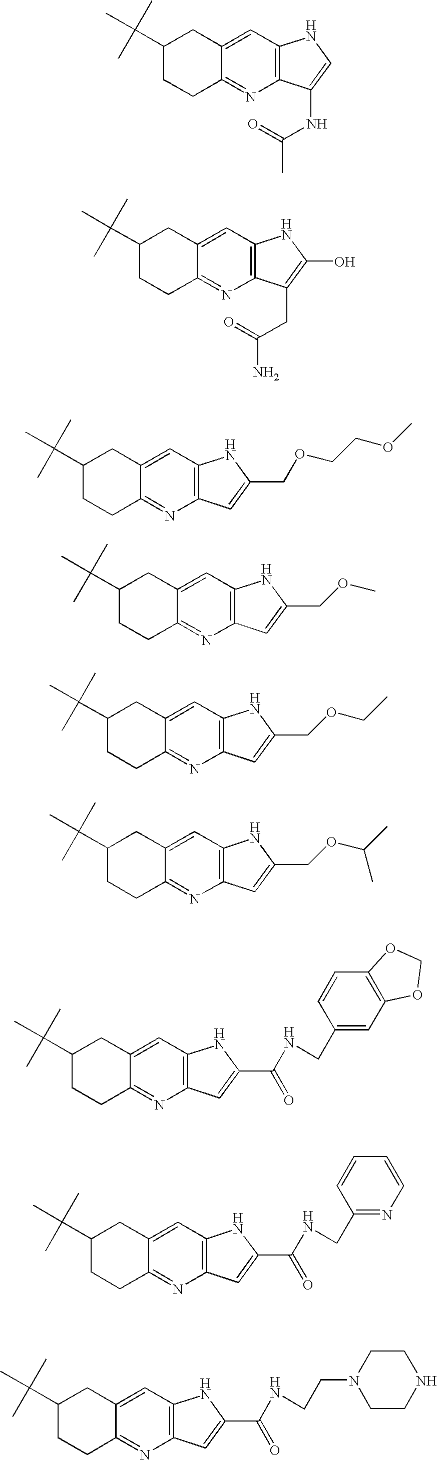 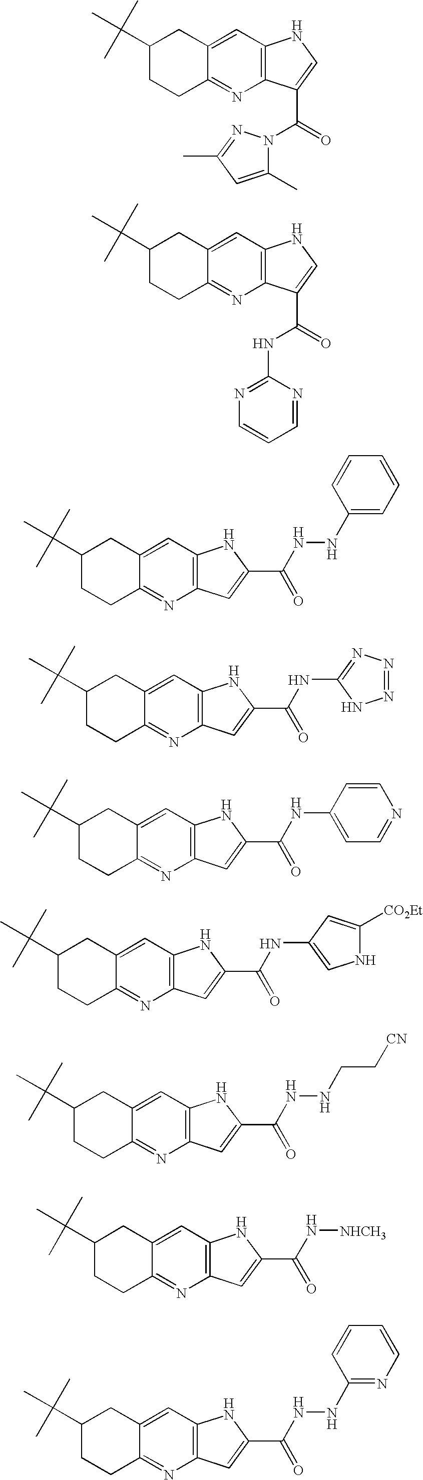 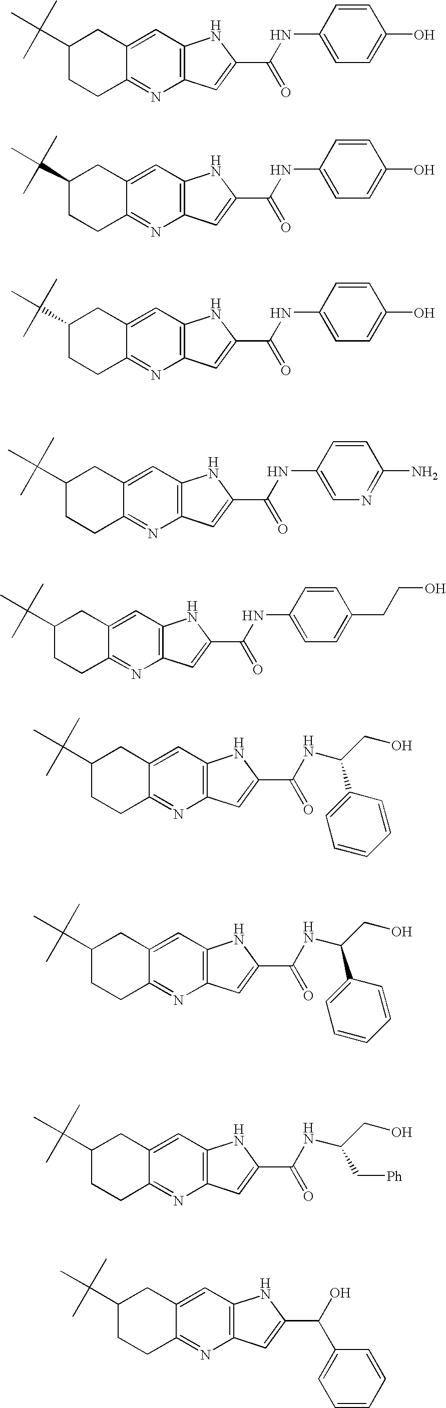 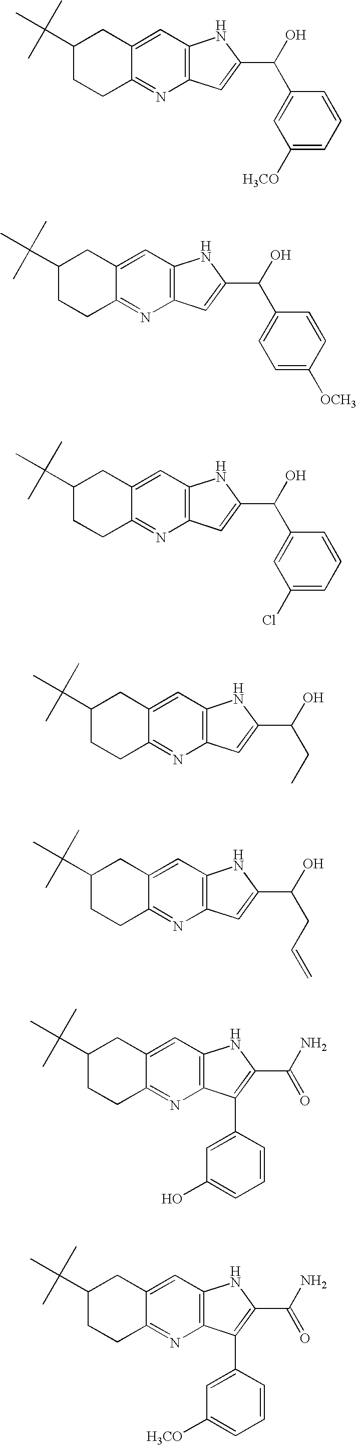 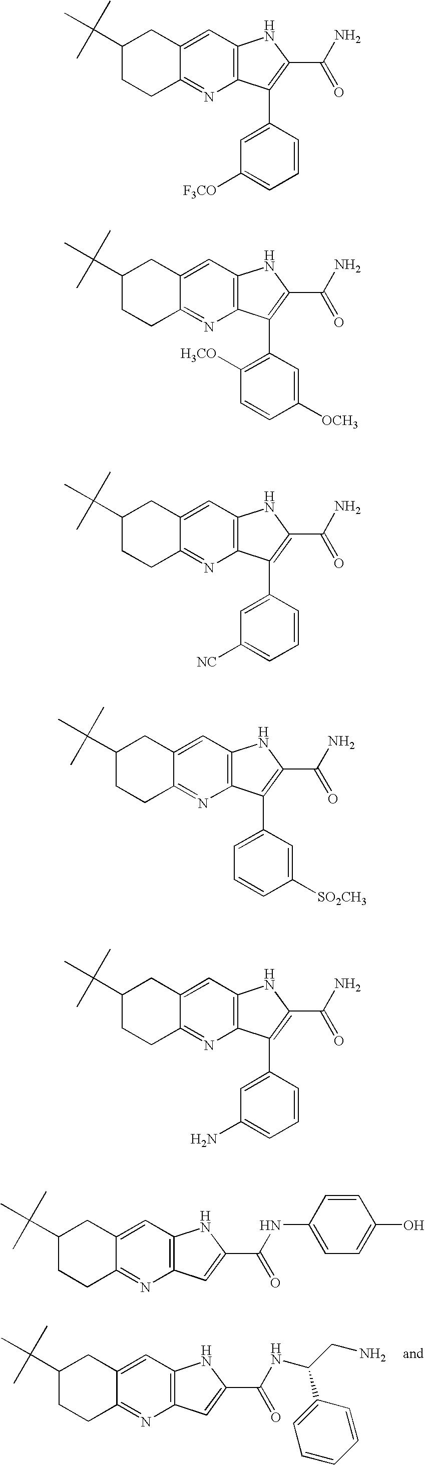 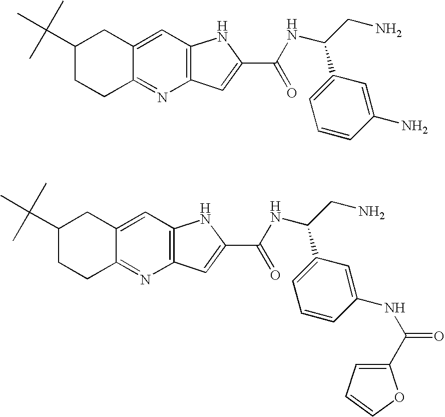
or a pharmaceutically acceptable salt, solvate or ester thereof.

19. The compound according to claim 18, selected from the group consisting of: 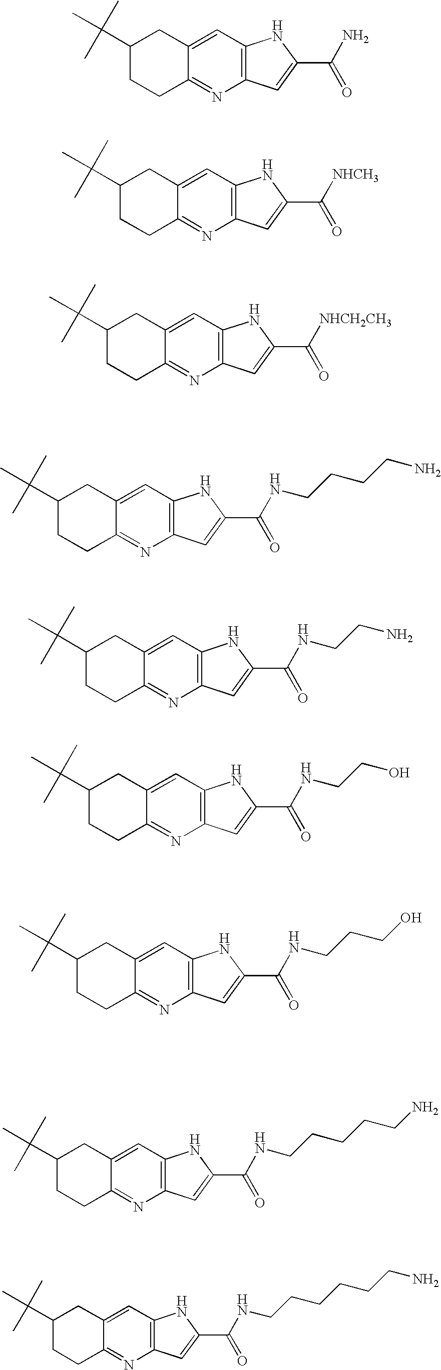 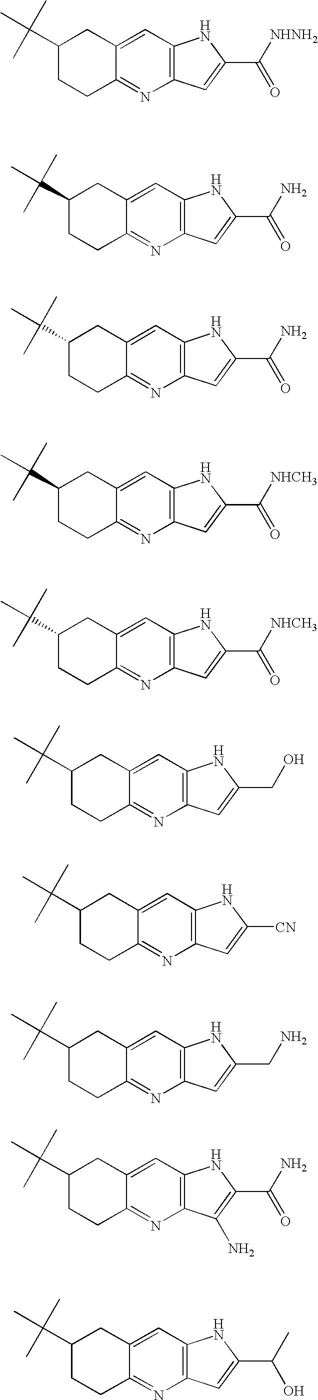 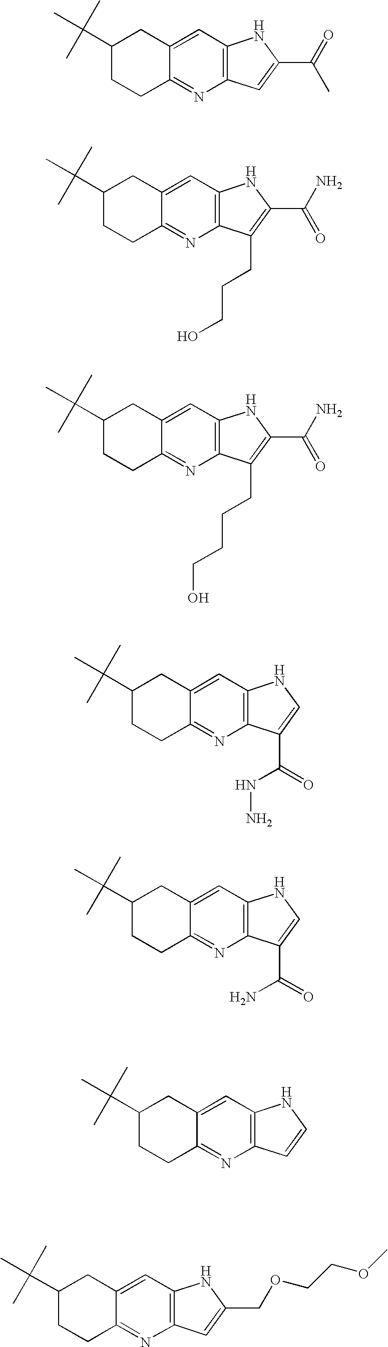 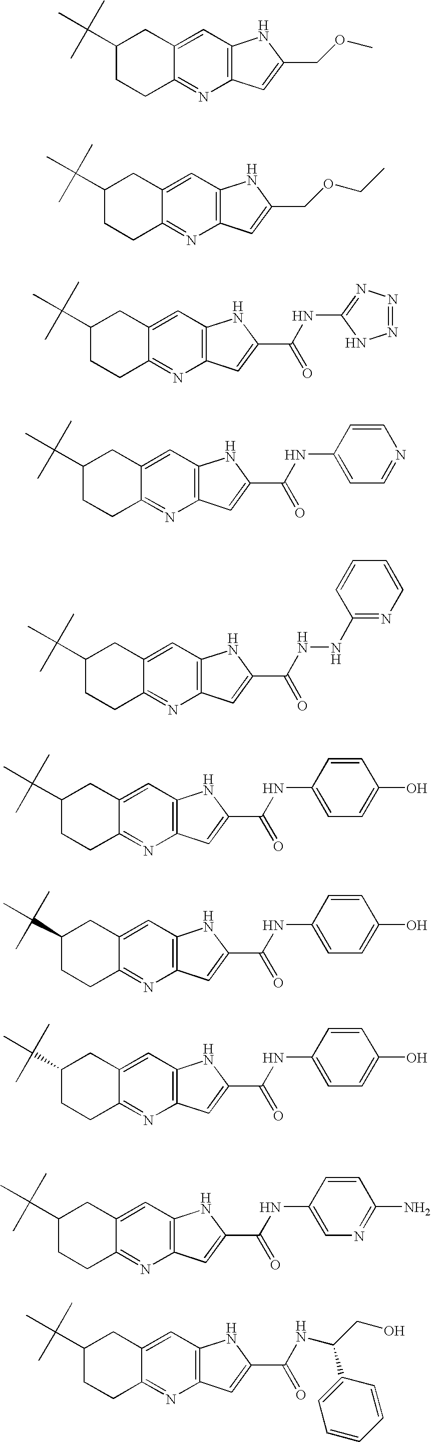 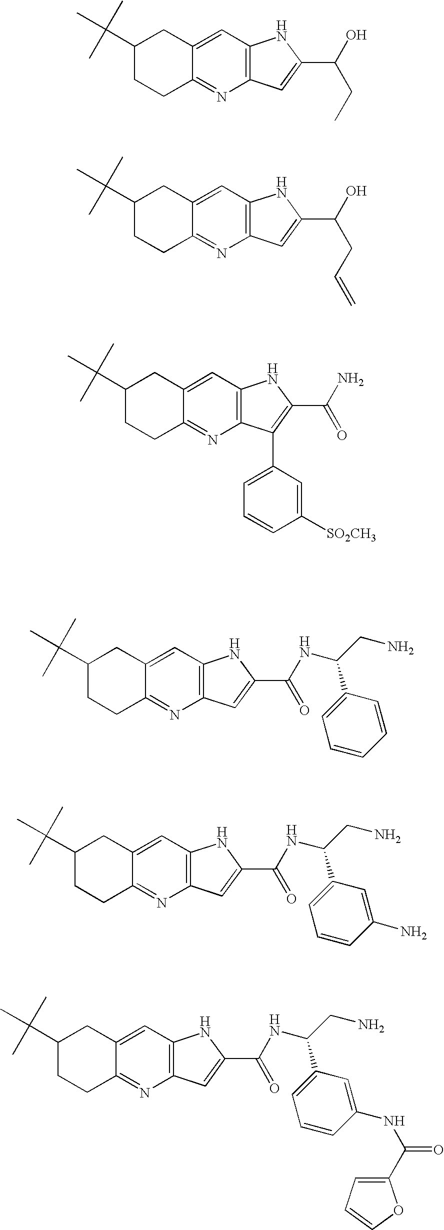
or a pharmaceutically acceptable salt, solvate or ester thereof.

20. An isolated or purified form of a compound of claim 1, or a pharmaceutically acceptable salt, solvate or ester thereof.

21. A pharmaceutical composition comprising a therapeutically effective amount of at least one compound of claim 1, or a pharmaceutically acceptable salt or ester thereof, in combination with a pharmaceutically acceptable carrier.

22. The pharmaceutical composition of claim 21, further comprising one or more compounds selected from the group consisting of an anti-cancer agent, a PPAR-γ agonist, a PPAR-δ agonist, an inhibitor of inherent multidrug resistance, an anti-emetic agent, and an immunologic-enhancing drug.

23. The pharmaceutical composition of claim 22, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

24. The pharmaceutical composition of claim 23, further comprising one or more anti-cancer agents selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, aurora kinase inhibitor, hormonal therapy combination, and aromatase combination.

25. The pharmaceutical composition of claim 24, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.

26. A method of inhibiting KSP activity in a subject in need thereof comprising administering to said subject an effective amount of at least one compound of claim 1, or a pharmaceutically acceptable salt, solvate or ester thereof.

27. A method of treating a cellular proliferative disease in a subject comprising administering to said subject in need of such treatment an effective amount of at least one compound of claim 1, or a pharmaceutically acceptable salt, solvate or ester thereof.

28. The method of claim 27, wherein the cellular proliferative disease is cancer, hyperplasia, cardiac hypertrophy, autoimmune diseases, fungal disorders, arthritis, graft rejection, inflammatory bowel disease, immune disorders, inflammation, cellular proliferation induced after medical procedures.

29. The method of claim 28, wherein the cancer is selected from cancers of the brain, genitourinary tract, cardiac, gastrointestinal, liver, bone, nervous system, and lung.

30. The method of claim 28, wherein the cancer is selected from lung adenocarcinoma, small cell lung cancer, pancreatic cancer, and breast carcinoma.

31. The method of claim 27, further comprising radiation therapy.

32. The method of claim 27, further comprising administering to the subject at least one compound selected from the group consisting of an anti-cancer agent, a PPAR-γ agonist, a PPAR-δ agonist, an inhibitor of inherent multidrug resistance, an anti-emetic agent, and an immunologic-enhancing drug.

33. The method of claim 32, wherein the disease is cancer.

34. The method of claim 33, further comprising radiation therapy.

35. The method of claim 32, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

36. The method of claim 32, further comprising one or more anti-cancer agent selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, hormonal therapy combination, and aromatase combination.

37. The method of claim 32, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole. Letrazole, Capecitabine, Reloxafine, Droloxafine, Hexamethylmelamine, doxorubicin, cyclophosphamide, gemcitabine, interferons, pegylated interferons, Erbitux and mixtures thereof.
